(19) United States
(12) Patent Application Publication
(10) Pub. No.: US 2017/0107183 A1
(43) Pub. Date: Apr. 20, 2017

(54) 2-(HETERO)ARYLPYRIDAZINONES AND THEIR USE AS HERBICIDES

(71) Applicant: BAYER CROPSCIENCE AKTIENGESELLSCHAFT, Monheim am Rhein (DE)

(72) Inventors: Hartmut AHRENS, Egelsbach (DE); Jörg TIEBES, Frankfurt (DE); Christian WALDRAFF, Bad Vilbel (DE); Hansjörg DIETRICH, Liederbach am Taunus (DE); Dirk SCHMUTZLER, Hattersheim (DE); Elmar GATZWEILER, Bad Nauheim (DE); Christopher ROSINGER, Hofheim (DE)

(21) Appl. No.: 15/312,597

(22) PCT Filed: May 19, 2015
(86) PCT No.: PCT/EP2015/060934

§ 371 Date: Nov. 18, 2016

(30) Foreign Application Priority Data
May 21, 2014 (EP) 14169229.3

(51) Int. Cl. C07D 237/16; A01N 43/58; C07D 237/18

(52) U.S. Cl. CPC C07D 237/16; C07D 237/18; A01N 43/58

(57) ABSTRACT

2-(Hetero)arylpyradazinones of the general formula (I) are described as herbicides.

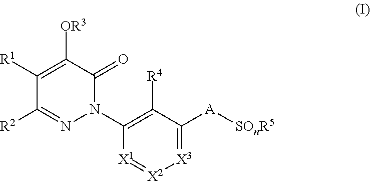

In this formula (I), R¹, R², R³, R⁴ and R⁵ are each radicals such as hydrogen, organic radicals such as alkyl, and other radicals such as halogen. X¹, X² and X³ represent nitrogen or an optionally substituted carbon atom.

The invention relates to the technical field of the herbicides, especially that of the herbicides for selective control of broad-leaved weeds and weed grasses in crops of useful plants.

WO02013/083774 A1 discloses pyridazinones as herbicides. Described in that publication are, inter alia, pyridazinones which carry, among others, a sulfonyl radical in a certain position of a heteroaryl ring. However, these active ingredients do not always exhibit sufficient activity against harmful plants and/or some do not have sufficient compatibility with some important crop plants such as cereal species, corn and rice.

It is an object of the present invention to provide alternative herbicidally active ingredients. This object is achieved by providing 2-(hetero)arylpyridazinones which carry a sulfur radical in a certain position of the (hetero)aryl ring.

The present invention thus provides 2-(hetero)arylpyridazinones of the formula (I) or salts thereof

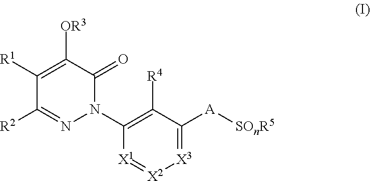

in which
R¹ represents hydrogen, halogen, cyano, (C₁-C₆)-alkyl, (C₃-C₆)-cycloalkyl, (C₂-C₆)-alkenyl, (C₄-C₆)-cycloalkenyl, (C₂-C₆)-alkynyl, halo-(C₁—C)-alkyl, (C₁-C₆)-alkoxy, (C₁-C₆)-alkoxy-(C₁-C₃)-alkyl, (C₁-C₆)-alkoxy-(C₂-C₆)-alkoxy, (C₁-C₆)-alkoxy-(C₂-C₆)-alkoxy-(C₁-C₃)-alkyl, (C₃-C₆)-cycloalkyl-(C₁-C₃)-alkyl, amino, (C₁-C₆)-alkylamino, di-(C₁-C₆)-alkylamino, (C₁-C₃)-alkyl-(O)C-amino-(C₁-C₄)-alkyl, (C₁-C₆)-alkyl-(O)ₙS, (C₁-C₆)-alkyl-(O)ₙS—(C₁-C₃)-alkyl, halo-(C₁-C₆)-alkyl-(O)ₙS or halo-(C₁-C₅)-alkyl-(O)ₙS—(C₁-C₃)-alkyl;
R² represents hydrogen, hydroxy, halogen, nitro, amino, cyano, (C₁-C₆)-alkyl, (C₁-C₃)-alkoxy, (C₃-C₆)-cycloalkyl, (C₂-C₆)-alkenyl, (C₂-C₆)-alkynyl, halo-(C₁-C₆)-alkyl, (C₁-C₆)-alkoxy-(C₁-C₃)-alkyl, (C₃-C₆)-cycloalkyl-(C₁-C₃)-alkyl, (C₁-C₆)-alkyl-(O)ₙS, (C₁-C₆)-alkyl-(O)ₙS—(C₁-C₃)-alkyl, halo-(C₁-C₆)-alkyl-(O)ₙS, halo-(C₁-C₅)-alkyl-(O)ₙS—(C₁-C₃)-alkyl, (C₁-C₃)-alkylamino or di-(C₁-C₃)-alkylamino;
R³ represents hydrogen, (C₁-C₆)-alkyl-(O)C, aryl-(O)C, (C₁-C₆)-alkoxy-(O)C, (C₁-C₆)-alkyl-(O)ₙS, (C₁-C₆)-alkyl-(O)ₙS(O)C or aryl-(O)ₙS, where the aryl groups are in each case substituted by s radicals R⁹;
R⁴ represents hydroxy, halogen, cyano, nitro, (C₁-C₆)-alkyl, (C₃-C₆)-cycloalkyl, halo-(C₁-C₆)-alkyl, (C₂-C₆)-alkenyl, halo-(C₂-C₆)-alkenyl, (C₂-C₆)-alkynyl, (C₁-C₆)-alkoxy, (C₂-C₆)-alkenyloxy, (C₃-C₆)-cycloalkyl-(C₁-C₃)-alkyl, (C₁-C₆)-alkoxy-(C₁-C₃)-alkyl, (C₁-C₆)-alkoxy-(C₂-C₆)-alkoxy, (C₁-C₆)-alkoxy-(C₂-C₆)-alkoxy-(C₁-C₃)-alkyl, halo-(C₁-C₆)-alkoxy, halo-(C₁-C₆)-alkoxy-(C₁-C₃)-alkyl, (C₁-C₆)-alkyl-(O)ₙS, halo-(C₁-C₆)-alkyl-(O)ₙS, aryl, aryl-(O)ₙS, heterocyclyl, heterocyclyl-(O)ₙS, aryloxy, aryl-(C₂-C₆)-alkyl, aryl-(C₁-C₆)-alkoxy, heterocyclyloxy, heterocyclyl-(C₁-C₃)-alkoxy-(C₁-C₃)-alkyl, HO(O)C, HO(O)C—(C₁-C₃)-alkoxy, (C₁-C₃)-alkoxy-(O)C, (C₁-C₃)-alkoxy-(O)C—(C₁-C₃)-alkoxy, (C₁-C₃)-alkylamino, di-(C₁-C₃)-alkylamino, (C₁-C₃)-alkylamino-(O)ₙS, (C₁-C₃)-alkylamino-(O)ₙS—(C₁-C₃)-alkyl, di-(C₁-C₃)-alkylamino-(O)ₙS, di-(C₁-C₃)-alkylamino-(O)ₙS—(C₁-C₃)-alkyl, (C₁-C₃)-alkylamino-(O)C, (C₁-C₃)-alkylamino-(O)C—(C₁-C₃)-alkyl, di-(C₁-C₃)-alkylamino-(O)C, di-(C₁-C₃)-alkylamino-(O)C—(C₁-C₃)-alkyl, (C₁-C₃)-alkyl-(O)C-amino, (C₁-C₃)-alkyl-(O)ₙS-amino, (C₁-C₃)-alkyl-(O)ₙS—(C₁-C₃)-alkylamino or (C₁-C₃)-alkyl-(O)ₙS-amino-(C₁-C₃)-alkyl, where the heterocyclyl groups and aryl groups are substituted by s radicals from the group consisting of (C₁-C₃)-alkyl, halo-(C₁-C₃)-alkyl, (C₁-C₃)-alkoxy, halo-(C₁-C₃)-alkoxy, phenyl, cyano, nitro and halogen;
A represents a direct bond or (C₁-C₄)-alkylene, where the methylene groups in (C₁-C₄)-alkylene independently of one another may carry n radicals from the group consisting of halogen, (C₁-C₄)-alkyl, halo-(C₁-C₄)-alkyl, (C₁-C₄)-alkoxy, halo-(C₁-C₄)-alkoxy or (C₁-C₄)-alkoxy-(C₁-C₄)-alkyl;
R⁵ represents (C₁-C₆)-alkyl, (C₃-C₆)-cycloalkyl, (C₃-C₆)-cycloalkyl-(C₁-C₆)-alkyl, (C₁-C₆)-alkoxy-(C₁-C₆)-alkyl;
X¹ represents N or CR⁶;
X² represents N or CR⁷;
X³ represents N or CR⁸;
R⁶ represents hydrogen, halogen, (C₁-C₃)-alkyl, (C₁-C₃)-alkoxy, (C₂-C₃)-alkenyl, (C₂-C₃)-alkynyl, halo-(C₁-C₃)-alkyl, halo-(C₁-C₃)-alkoxy;
R⁷ represents hydrogen, halogen, (C₁-C₃)-alkyl;
R⁸ represents hydrogen, hydroxy, halogen, cyano, nitro, (C₁-C₆)-alkyl, (C₃-C₆)-cycloalkyl, halo-(C₁-C₆)-alkyl, (C₂-C₆)-alkenyl, halo-(C₂-C₆)-alkenyl, (C₂-C₆)-alkynyl, (C₁-C₆)-alkoxy, (C₂-C₆)-alkenyloxy, (C₃-C₅)-cycloalkyl-(C₁-C₃)-alkyl, (C₁-C₆)-alkoxy-(C₁-C₃)-alkyl, (C₁-C₆)-alkoxy-(C₂-C₆)-alkoxy, (C₁-C₆)-alkoxy-(C₂-C₆)-alkoxy-(C₁-C₃)-alkyl, halo-(C₁-C₆)-alkoxy, halo-(C₁-C₆)-alkoxy-(C₁-C₃)-alkyl, (C₁-C₆)-alkyl-(O)ₙS, halo-(C₁-C₆)-alkyl-(O)ₙS, aryl, aryl-(O)ₙS, heterocyclyl, heterocyclyl-(O)ₙS, aryloxy, aryl-(C₂-C₆)-alkyl, aryl-(C₁-C₆)-alkoxy, heterocyclyloxy, heterocyclyl-(C₁-C₃)-alkoxy-(C₁-C₃)-alkyl, HO(O)C, HO(O)C—(C₁-C₃)-alkoxy, (C₁-C₃)-alkoxy-(O)C, (C₁-C₃)-alkoxy-(O)C—(C₁-C₃)-alkoxy, (C₁-C₃)-alkylamino, di-(C₁-C₃)-alkylamino, (C₁-C₃)-alkylamino-(O)ₙS, (C₁-C₃)-alkylamino-(O)ₙS—(C₁-C₃)-alkyl, di-(C₁-C₃)-alkylamino-(O)ₙS, di-(C₁-C₃)-alkylamino-(O)ₙS—(C₁-C₃)-alkyl, (C₁-C₃)-alkylamino-(O)C, (C₁-C₃)-alkylamino-(O)C—(C₁-C₃)-alkyl, di-(C₁-C₃)-alkylamino-(O)C, di-(C₁-C₃)-alkylamino-(O)C—(C₁-C₃)-alkyl, (C₁-C₃)-alkyl-(O)C-amino, (C₁-C₃)-alkyl-(O)ₙS-amino, (C₁-C₃)-alkyl-(O)ₙS—(C₁-C₃)-alkylamino or (C₁-C₃)-alkyl-(O)ₙS-amino-(C₁-C₃)-alkyl, where the heterocyclyl groups and aryl groups are substituted by s radicals from the group consisting of (C₁-C₃)-alkyl, halo-(C₁-C₃)-alkyl, (C₁-C₃)-alkoxy, halo-(C₁-C₃)-alkoxy, (C₁-C₆)-alkyl-(O)ₙS, phenyl, cyano, nitro and halogen,
or
R⁷ and R⁸ together with the carbon atoms to which they are attached represent an unsaturated five- or six-membered ring which contains s nitrogen atoms and is substituted by s radicals R¹⁰;
R⁹ represents halogen, (C₁-C₃)-alkyl, halo-(C₁-C₃)-alkyl, (C₁-C₆)-alkoxy,
R¹⁰ represents cyano, halogen, (C₁-C₃)-alkyl-(O)ₙS, (C₁-C₃)-alkyl, (C₂-C₃)-alkenyl, (C₂-C₃)-alkynyl, halo-(C₁-C₃)-alkyl or morpholinyl;
n represents 0, 1 or 2;
s represents 0, 1, 2 or 3,
with the proviso that R⁵ does not represent (C₁-C₆)-alkyl if A represents a direct bond.

In the formula (I) and all the formulae which follow, alkyl radicals having more than two carbon atoms may be straight-chain or branched. Alkyl radicals are, for example, methyl, ethyl, n-propyl or isopropyl, n-, iso-, t- or 2-butyl, pentyls, hexyls such as n-hexyl, isohexyl and 1,3-dimethylbutyl. Analogously, alkenyl is, for example, allyl, 1-methylprop-2-en-1-yl, 2-methylprop-2-en-1-yl, but-2-en-1-yl, but-3-en-1-yl, 1-methylbut-3-en-1-yl and 1-methylbut-2-en-1-yl. Alkynyl is, for example, propargyl, but-2-yn-1-yl, but-3-yn-1-yl, 1-methylbut-3-yn-1-yl. The multiple bond may be in any position in each unsaturated radical. Cycloalkyl is a carbocyclic saturated ring system having three to six carbon atoms, for example cyclopropyl, cyclobutyl, cyclopentyl or cyclohexyl. Analogously, cycloalkenyl is a monocyclic alkenyl group having three to six carbon ring members, for example cyclopropenyl, cyclobutenyl, cyclopentenyl and cyclohexenyl, where the double bond may be in any position.

Halogen represents fluorine, chlorine, bromine or iodine.

Heterocyclyl is a saturated, partially saturated, fully unsaturated or aromatic cyclic radical which contains 3 to 6 ring atoms, 1 to 4 of which are from the group consisting of oxygen, nitrogen and sulfur, and which may additionally be fused by a benzo ring. For example, heterocyclyl represents piperidinyl, pyrrolidinyl, morpholinyl, tetrahydrofuranyl, dihydrofuranyl, oxetanyl, benzimidazol-2-yl, furanyl, imidazolyl, isoxazolyl, isothiazolyl, oxazolyl, pyrazinyl, pyrimidinyl, pyridazinyl, pyridinyl, benzisoxazolyl, thiazolyl, pyrrolyl, pyrazolyl, thiophenyl, 1,2,3-oxadiazolyl, 1,2,4-oxadiazolyl, 1,2,5-oxadiazolyl, 1,3,4-oxadiazolyl, 1,2,4-triazolyl, 1,2,3-triazolyl, 1,2,5-triazolyl, 1,3,4-triazolyl, 1,2,4-triazolyl, 1,2,4-thiadiazolyl, 1,3,4-thiadiazolyl, 1,2,3-thiadiazolyl, 1,2,5-thiadiazolyl, 2H-1,2,3,4-tetrazolyl, 1H-1,2,3,4-tetrazolyl, 1,2,3,4-oxatriazolyl, 1,2,3,5-oxatriazolyl, 1,2,3,4-thiatriazolyl and 1,2,3,5-thiatriazolyl.

Aryl is phenyl or naphthyl.

If a group is polysubstituted by radicals, this is to be understood as meaning that this group is substituted by one or more identical or different radicals selected from the radicals mentioned.

Depending on the nature of the substituents and the manner in which they are attached, the compounds of the general formula (I) may be present as stereoisomers. If, for example, one or more asymmetrically substituted carbon atoms are present, there may be enantiomers and diastereomers. Stereoisomers likewise occur when n represents 1 (sulfoxides). Stereoisomers can be obtained from the mixtures obtained in the preparation by customary separation methods, for example by chromatographic separation processes. It is likewise possible to selectively prepare stereoisomers by using stereoselective reactions with use of optically active starting materials and/or auxiliaries. The invention also relates to all the stereoisomers and mixtures thereof that are encompassed by the general formula (I) but are not defined specifically.

The compounds of the formula (I) are capable of forming salts. Salts may be formed by action of a base on compounds of the formula (I). Examples of suitable bases are organic amines such as trialkylamines, morpholine, piperidine and pyridine, and the hydroxides, carbonates and hydrogencarbonates of ammonium, alkali metals or alkaline earth metals, especially sodium hydroxide, potassium hydroxide, sodium carbonate, potassium carbonate, sodium hydrogencarbonate and potassium hydrogencarbonate. These salts are compounds in which the acidic hydrogen is replaced by an agriculturally suitable cation, for example metal salts, especially alkali metal salts or alkaline earth metal salts, in particular sodium and potassium salts, or else ammonium salts, salts with organic amines or quaternary ammonium salts, for example with cations of the formula [NR^{a}R^{b}R^{c}R^{d}]+ in which R^{a} to R^{d} are each independently an organic radical, especially alkyl, aryl, aralkyl or alkylaryl. Also suitable are alkylsulfonium and alkylsulfoxonium salts, such as (C₁-C₄)-trialkylsulfonium and (C₁-C₄)-trialkylsulfoxonium salts.

Preference is given to compounds of the general formula (I) in which

R¹ represents hydrogen, halogen, cyano, (C₁-C₆)-alkyl, (C₃-C₆)-cycloalkyl, (C₂-C₆)-alkenyl, (C₂-C₆)-alkynyl, halo-(C₁-C₆)-alkyl, (C₁-C₆)-alkoxy-(C₁-C₃)-alkyl, (C₃-C₆)-cycloalkyl-(C₁-C₃)-alkyl, amino or (C₁-C₆)-alkyl-(O)ₙS;
R² represents hydrogen, halogen, cyano, (C₁-C₆)-alkyl, (C₂-C₆)-alkenyl, (C₂-C₆)-alkynyl, halo-(C₁-C₃)-alkyl or (C₁-C₆)-alkyl-(O)ₙS;
R³ represents hydrogen,
R⁴ represents hydroxy, halogen, cyano, nitro, (C₁-C₆)-alkyl, (C₃-C₆)-cycloalkyl, halo-(C₁-C₆)-alkyl, (C₂-C₆)-alkenyl, (C₂-C₆)-alkynyl, (C₁-C₆)-alkoxy, (C₁-C₅)-alkoxy-(C₁-C₃)-alkyl, (C₁-C₆)-alkoxy-(C₂-C₆)-alkoxy, (C₁-C₆)-alkoxy-(C₂-C₆)-alkoxy-(C₁-C₃)-alkyl, halo-(C₁-C₆)-alkoxy, halo-(C₁-C₆)-alkoxy-(C₁-C₃)-alkyl, (C₁-C₆)-alkyl-(O)ₙS, halo-(C₁-C₆)-alkyl-(O)ₙS, aryl, heterocyclyl, aryloxy, heterocyclyl-(C₁-C₃)-alkoxy-(C₁-C₃)-alkyl, (C₁-C₃)-alkylamino, di-(C₁-C₃)-alkylamino, (C₁-C₃)-alkylamino-(O)ₙS, (C₁-C₃)-alkylamino-(O)ₙS—(C₁-C₃)-alkyl, di-(C₁-C₃)-alkylamino-(O)ₙS, di-(C₁-C₃)-alkylamino-(O)ₙS—(C₁-C₃)-alkyl, (C₁-C₃)-alkylamino-(O)C, di-(C₁-C₃)-alkylamino-(O)C, di-(C₁-C₃)-alkylamino-(O)C—(C₁-C₃)-alkyl, (C₁-C₃)-alkyl-(O)C-amino or (C₁-C₃)-alkyl-(O)ₙS-amino, where the heterocyclyl groups and aryl groups are substituted by s radicals from the group consisting of (C₁-C₃)-alkyl, halo-(C₁-C₃)-alkyl, (C₁-C₃)-alkoxy, halo-(C₁-C₃)-alkoxy, cyano, nitro and halogen;
A represents a direct bond or (C₁-C₄)-alkylene;
R⁵ represents (C₁-C₆)-alkyl, (C₃-C₆)-cycloalkyl, (C₃-C₆)-cycloalkyl-(C₁-C₆)-alkyl, (C₁-C₆)-alkoxy-(C₁-C₆)-alkyl;
X¹ represents CR⁶;
X² represents CR⁷;
X³ represents CR⁸;
R⁶ and R⁷ independently of one another represent hydrogen, halogen or (C₁-C₃)-alkyl;
R⁸ represents hydrogen, halogen, nitro, (C₁-C₆)-alkyl, (C₃-C₆)-cycloalkyl, halo-(C₁-C₆)-alkyl, (C₂-C₆)-alkenyl, halo-(C₂-C₆)-alkenyl, (C₂-C₆)-alkynyl, (C₁-C₆)-alkoxy, (C₂-C₆)-alkenyloxy, (C₃-C₆)-cycloalkyl-(C₁-C₃)-alkyl, (C₁-C₆)-alkoxy-(C₁-C₃)-alkyl, (C₁-C₆)-alkoxy-(C₂-C₆)-alkoxy, halo-(C₁-C₆)-alkoxy, (C₁-C₆)-alkyl-(O)ₙS or phenyl, where the phenyl group is substituted by s radicals from the group consisting of (C₁-C₃)-alkyl, halo-(C₁-C₃)-alkyl, (C₁-C₃)-alkoxy, halo-(C₁-C₃)-alkoxy, (C₁-C₆)-alkyl-(O)ₙS, phenyl, cyano, nitro and halogen;
n represents 0, 1 or 2;
s represents 0, 1, 2 or 3,
with the proviso that R⁵ does not represent (C₁-C₆)-alkyl if A represents a direct bond.

Particular preference is given to compounds of the general formula (I) in which

R¹ represents hydrogen, amino, chlorine, bromine, cyano, methyl, ethyl, isopropyl, cyclopropyl, vinyl, propargyl, isopropenyl or methyl-(O)ₙS;
R² represents hydrogen, halogen or (C₁-C₆)-alkyl;
R³ represents hydrogen;
R⁴ represents fluorine, chlorine, cyano, nitro, methyl, trifluoromethyl, 2-fluoroethyl, methoxyethoxymethyl, trifluoromethoxymethyl, methyl-(O)ₙS, aryl, isoxazolinyl, morpholinyl or methyl-(O)ₙS-amino, where the heterocyclyl groups and aryl groups are substituted by s radicals from the group consisting of methyl, trifluoromethyl and chlorine;
A represents a direct bond or (C₁-C₄)-alkylene;
R⁵ represents (C₁-C₆)-alkyl, (C₃-C₆)-cycloalkyl, (C₃-C₆)-cycloalkyl-(C₁-C₆)-alkyl, (C₁-C₆)-alkoxy-(C₁-C₆)-alkyl;
X¹ represents CR⁶;
X² represents CR⁷;
X³ represents CR⁶;
R⁶ and R⁷ represent hydrogen;
R⁸ represents hydrogen, halogen, (C₁-C₆)-alkyl, halo-(C₁-C₆)-alkyl, (C₂-C₅)-alkenyl, (C₂-C₆)-alkynyl or (C₁-C₆)-alkyl-(O)ₙS;
n represents 0, 1 or 2;
s represents 0, 1, 2 or 3,
with the proviso that R⁵ does not represent (C₁-C₆)-alkyl if A represents a direct bond.

Very particular preference is given to compounds of the general formula (I) in which

R¹ represents methyl or vinyl;
R² represents hydrogen;
R³ represents hydrogen,
R⁴ represents methyl, chlorine, trifluoromethyl or methyl-(O)ₙS;
A represents a direct bond, —CH₂— or —CH₂CH₂—;
R⁵ represents methyl, ethyl, cyclopropyl, cyclopropylmethyl, methoxyethyl;
X¹ represents CR⁶;
X² represents CR⁷;
X³ represents CR⁸;
R⁶ and R⁷ represent hydrogen,
R⁸ represents methyl, ethyl, chlorine, trifluoromethyl or methyl(O)ₙS;
n represents 0, 1 or 2,
with the proviso that R⁵ does not represent methyl or ethyl if A represents a direct bond.

In all the formulae specified hereinafter, the substituents and symbols have the same meaning as described in formula (I), unless defined differently.

Compounds according to the invention can be prepared, for example, analogously to the methods specified in WO 2013/083774 A1.

The hydrazines on which the compounds according to the invention are based can be prepared by methods well known in the literature. A review can be found, for example, in Houben-Weyl, Methoden der Organischen Chemie [Methods of Organic Chemistry], Georg Thieme Verlag Stuttgart, Vol. E 16a, part 1, expanded and supplementary volumes to the fourth edition 1990, p. 648 ff. and p. 678 ff.

Thioethers of the formula (I) where n=0 can be oxidized to give the corresponding sulfoxides or sulfones. Oxidation methods leading, in a targeted manner, to the sulfoxide or sulfone are known from the literature. A number of oxidation systems are suitable, for example peracids such as meta-chloroperbenzoic acid, which is optionally generated in situ (for example peracetic acid in the system acetic acid/hydrogen peroxide/sodium tungstate(VI)) (Houben-Weyl, Methoden der Organischen Chemie [Methods of Organic Chemistry], Georg Thieme Verlag Stuttgart, Vol. E 11, expanded and supplementary volumes to the 4th edition 1985, p. 702 ff., p. 718 ff. and p. 1194 ff.). At which stage of the synthesis cascade the oxidation of the thioether is expedient depends inter alia on the substitution pattern and the oxidizing agent.

The workup of the respective reaction mixtures is generally effected by known processes, for example by crystallization, aqueous-extractive workup, by chromatographic methods or by a combination of these methods.

Collections of compounds of the formula (I) and/or salts thereof which can be synthesized by the abovementioned reactions can also be prepared in a parallelized manner, in which case this may be accomplished in a manual, partly automated or fully automated manner. It is possible, for example, to automate the conduct of the reaction, the work-up or the purification of the products and/or intermediates. Overall, this is understood to mean a procedure as described, for example, by D. Tiebes in Combinatorial Chemistry—Synthesis, Analysis, Screening (editor Günther Jung), Wiley, 1999, on pages 1 to 34.

For the parallelized conduct of the reaction and workup, it is possible to use a number of commercially available instruments, for example Calypso reaction blocks from Barnstead International, Dubuque, Iowa 52004-0797, USA or reaction stations from Radleys, Shirehill, Saffron Walden, Essex, CB11 3AZ, England, or MultiPROBE Automated Workstations from PerkinElmer, Waltham, Mass. 02451, USA. For the parallelized purification of compounds of the general formula (I) and salts thereof or of intermediates which occur in the course of preparation, available apparatuses include chromatography apparatuses, for example from ISCO, Inc., 4700 Superior Street, Lincoln, Nebr. 68504, USA.

The apparatuses detailed lead to a modular procedure in which the individual working steps are automated, but manual operations have to be carried out between the working steps. This can be circumvented by using partly or fully integrated automation systems in which the respective automation modules are operated, for example, by robots. Automation systems of this type can be obtained, for example, from Caliper, Hopkinton, Mass. 01748, USA.

The implementation of single or multiple synthesis steps can be supported by the use of polymer-supported reagents/scavenger resins. The specialist literature describes a series of experimental protocols, for example in ChemFiles, Vol. 4, No. 1, Polymer-Supported Scavengers and Reagents for Solution-Phase Synthesis (Sigma-Aldrich).

Aside from the methods described here, compounds of the general formula (I) and salts thereof can be prepared completely or partially by solid-phase-supported methods. For this purpose, individual intermediates or all intermediates in the synthesis or a synthesis adapted for the corresponding procedure are bound to a synthesis resin. Solid-phase-supported synthesis methods are described adequately in the technical literature, for example Barry A. Bunin in “The Combinatorial Index”, Academic Press, 1998 and Combinatorial Chemistry—Synthesis, Analysis, Screening (editor: Gunther Jung), Wiley, 1999. The use of solid-phase-supported synthesis methods permits a number of protocols, which are known from the literature and which for their part may be performed manually or in an automated manner. The reactions can be performed, for example, by means of IRORI technology in microreactors from Nexus Biosystems, 12140 Community Road, Poway, Calif. 92064, USA.

Both in the solid and in the liquid phase, the conduction of individual or several synthesis steps may be supported by the use of microwave technology. The specialist literature describes a series of experimental protocols, for example in Microwaves in Organic and Medicinal Chemistry (editor: C. O. Kappe and A. Stadler), Wiley, 2005.

The preparation by the processes described here gives compounds of the formula (I) and salts thereof in the form of substance collections, which are called libraries. The present invention also provides libraries comprising at least two compounds of the formula (I) and salts thereof.

The compounds of the invention have excellent herbicidal efficacy against a broad spectrum of economically important mono- and dicotyledonous annual harmful plants.

The active ingredients also act efficiently on perennial weeds which produce shoots from rhizomes, root stocks and other perennial organs and which are difficult to control.

The present invention therefore also, provides a method for controlling unwanted plants or for regulating the growth of plants, preferably in plant crops, in which one or more compound(s) according to the invention is/are applied to the plants (for example harmful plants such as monocotyledonous or dicotyledonous weeds or unwanted crop plants), the seed (for example grains, seeds or vegetative propagules such as tubers or shoot parts with buds) or the area on which the plants grow (for example the area under cultivation). The compounds of the invention can be deployed, for example, prior to sowing (if appropriate also by incorporation into the soil), prior to emergence or after emergence. Specific examples of some representatives of the monocotyledonous and dicotyledonous weed flora which can be controlled by the compounds of the invention are as follows, though there is no intention to restrict the enumeration to particular species.

Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria and Sorghum.

Dicotyledonous weeds of the genera: Abutilon, Amaranthus, Ambrosia, Anoda, Anthemis, Aphanes, Artemisia, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola and Xanthium.

If the compounds of the invention are applied to the soil surface before germination, either the emergence of the weed seedlings is prevented completely or the weeds grow until they have reached the cotyledon stage, but then they stop growing and ultimately die completely after three to four weeks have passed.

If the active ingredients are applied post-emergence to the green parts of the plants, growth stops after the treatment, and the harmful plants remain at the growth stage at the time of application, or they die completely after a certain time, such that competition by the weeds, which is harmful to the crop plants, is thus eliminated very early and in a lasting manner.

Although the compounds of the invention have outstanding herbicidal activity against monocotyledonous and dicotyledonous weeds, crop plants of economically important crops, for example dicotyledonous crops of the genera Arachis, Beta, Brassica, Cucumis, Cucurbita, Helianthus, Daucus, Glycine, Gossypium, Ipomoea, Lactuca, Linum, Lycopersicon, Miscanthus, Nicotiana, Phaseolus, Pisum, Solanum, Vicia, or monocotyledonous crops of the genera Allium, Ananas, Asparagus, Avena, Hordeum, Oryza, Panicum, Saccharum, Secale, Sorghum, Triticale, Triticum, Zea, in particular 15 Zea and Triticum, will be damaged to a negligible extent only, if at all, depending on the structure of the particular compound of the invention and its application rate. For these reasons, the present compounds are very suitable for selective control of unwanted plant growth in plant crops such as agriculturally useful plants or ornamental plants.

In addition, the compounds of the invention, depending on their particular chemical structure and the application rate deployed, have outstanding growth-regulating properties in crop plants. They intervene in the plants' own metabolism with regulatory effect, and can thus be used for controlled influencing of plant constituents and to facilitate harvesting, for example by triggering desiccation and stunted growth. In addition, they are also suitable for general control and inhibition of unwanted vegetative growth without killing the plants. Inhibition of vegetative growth plays a major role for many mono- and dicotyledonous crops since, for example, this can reduce or completely prevent lodging.

By virtue of their herbicidal and plant growth regulatory properties, the active ingredients can also be used to control harmful plants in crops of genetically modified plants or plants modified by conventional mutagenesis. In general, transgenic plants are characterized by particular advantageous properties, for example by resistances to certain pesticides, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material.

It is preferable with a view to transgenic crops to use the compounds of the invention in economically important transgenic crops of useful plants and ornamentals, for example of cereals such as wheat, barley, rye, oats, millet, rice and corn or else crops of sugar beet, cotton, soybean, oilseed rape, potato, manioc, tomato, peas and other vegetables.

Preferably, the compounds of the invention can be used as herbicides in crops of useful plants which are resistant, or have been made resistant by genetic engineering, to the phytotoxic effects of the herbicides.

Conventional ways of producing novel plants which have modified properties in comparison to existing plants consist, for example, in traditional cultivation methods and the generation of mutants. Alternatively, novel plants with modified properties can be generated with the aid of recombinant methods (see, for example, EP-A-0221044, EP-A-0131624). For example, there have been descriptions in several cases of:
genetic modifications of crop plants for the purpose of modifying the starch synthesized in the plants (e.g. WO 92/11376, WO 92/14827, WO 91/19806),
transgenic crop plants which are resistant to particular herbicides of the glufosinate type (cf., for example, EP-A-0242236, EP-A-242246) or glyphosate type
   (WO 92/00377) or the sulfonylurea type (EP-A-0257993, US A 5013659),
transgenic crop plants, for example cotton, capable of producing Bacillus thuringiensis toxins (Bt toxins), which make the plants resistant to particular pests (EP-A-0142924,
   EP-A-0193259),
transgenic crop plants with a modified fatty acid composition (WO 91/13972),
genetically modified crop plants with novel constituents or secondary metabolites, for example novel phytoalexins, which bring about an increased disease resistance (EPA 309862, EPA0464461),
genetically modified plants having reduced photorespiration, which have higher yields and higher stress tolerance (EPA 0305398),
transgenic crop plants which produce pharmaceutically or diagnostically important proteins (“molecular pharming”),
transgenic crop plants which feature higher yields or better quality,
transgenic crop plants which feature a combination, for example, of the abovementioned novel properties (“gene stacking”).

Numerous molecular biology techniques which can be used to produce novel transgenic plants with modified properties are known in principle; see, for example, I. Potrykus and G. Spangenberg (eds.) Gene Transfer to Plants, Springer Lab Manual (1995), Springer Verlag Berlin, Heidelberg, or Christou, “Trends in Plant Science” 1 (1996) 423-431).

For such recombinant manipulations, nucleic acid molecules which allow mutagenesis or sequence alteration by recombination of DNA sequences can be introduced into plasmids. With the aid of standard methods, it is possible, for example, to undertake base exchanges, remove parts of sequences or add natural or synthetic sequences. To join the DNA fragments with one another, adapters or linkers can be placed onto the fragments, see e.g. Sambrook et al., 1989, Molecular Cloning, A Laboratory Manual, 2nd edition Cold Spring Harbor Laboratory Press, Cold Spring Harbor, N.Y., or Winnacker “Gene und Klone [Genes and clones]”, VCH Weinheim 2nd edition 1996.

For example, the generation of plant cells with a reduced activity of a gene product can be achieved by expressing at least one corresponding antisense RNA, a sense RNA for achieving a cosuppression effect, or by expressing at least one suitably constructed ribozyme which specifically cleaves transcripts of the abovementioned gene product. To this end, it is firstly possible to use DNA molecules which encompass the entire coding sequence of a gene product inclusive of any flanking sequences which may be present, and also DNA molecules which only encompass portions of the coding sequence, in which case it is necessary for these portions to be long enough to have an antisense effect in the cells. It is also possible to use DNA sequences which have a high degree of homology to the coding sequences of a gene product, but are not completely identical to them.

When expressing nucleic acid molecules in plants, the protein synthesized may be localized in any desired compartment of the plant cell. However, to achieve localization in a particular compartment, it is possible, for example, to join the coding region to DNA sequences which ensure localization in a particular compartment. Such sequences are known to those skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106). The nucleic acid molecules can also be expressed in the organelles of the plant cells.

The transgenic plant cells can be regenerated by known techniques to give rise to entire plants. In principle, the transgenic plants may be plants of any desired plant species, i.e. not only monocotyledonous but also dicotyledonous plants.

Thus, transgenic plants can be obtained whose properties are altered by overexpression, suppression or inhibition of homologous (=natural) genes or gene sequences or expression of heterologous (=foreign) genes or gene sequences.

The compounds of the invention can be used with preference in transgenic crops which are resistant to growth regulators, for example dicamba, or to herbicides which inhibit essential plant enzymes, for example acetolactate synthases (ALS), EPSP synthases, glutamine synthases (GS) or hydroxyphenylpyruvate dioxygenases (HPPD), or to herbicides from the group of the sulphonylureas, the glyphosates, glufosinates or benzoylisoxazoles and analogous active ingredients.

When the active ingredients of the invention are used in transgenic crops, not only do the effects toward harmful plants which are observed in other crops occur, but often also effects which are specific to application in the particular transgenic crop, for example an altered or specifically widened spectrum of weeds which can be controlled, altered application rates which can be used for the application, preferably good combinability with the herbicides to which the transgenic crop is resistant, and influencing of growth and yield of the transgenic crop plants.

The invention therefore also provides for the use of the compounds of the invention as herbicides for control of harmful plants in transgenic crop plants.

The compounds of the invention can be applied in the form of wettable powders, emulsifiable concentrates, sprayable solutions, dusting products or granules in the customary formulations. The invention therefore also provides herbicidal and plant-growth-regulating compositions which comprise the compounds of the invention.

The compounds of the invention can be formulated in various ways, according to the biological and/or physicochemical parameters required. Possible formulations include, for example: Wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), dispersions based on oil or water, oil-miscible solutions, capsule suspensions (CS), dusting products (DP), dressings, granules for scattering and soil application, granules (GR) in the form of micro granules, spray granules, absorption and adsorption granules, water-dispersible granules (WG), water-soluble granules (SG), ULV formulations, microcapsules and waxes.

These individual formulation types are known in principle and are described, for example, in: Winnacker-Küchler, “Chemische Technologie” [Chemical Technology], volume 7, C. Hanser Verlag Munich, 4th Ed. 1986, Wade van Valkenburg, “Pesticide Formulations”, Marcel Dekker, N.Y., 1973, K. Martens, “Spray Drying” Handbook, 3rd Ed. 1979, G. Goodwin Ltd. London.

The formulation auxiliaries required, such as inert materials, surfactants, solvents and further additives, are likewise known and are described, for example, in: Watkins, “Handbook of Insecticide Dust Diluents and Carriers”, 2nd ed., Darland Books, Caldwell N.J.; H. v. Olphen, “Introduction to Clay Colloid Chemistry”, 2nd ed., J. Wiley & Sons, N.Y.; C. Marsden, “Solvents Guide”, 2nd ed., Interscience, N.Y. 1963; McCutcheon's “Detergents and Emulsifiers Annual”, MC Publ. Corp., Ridgewood N.J., Sisley and Wood, “Encyclopedia of Surface Active Agents”, Chem. Publ. Co. Inc., N.Y. 1964; Schönfeldt, “Grenzflachenaktive Äthylenoxidaddukte” [Interface-active Ethylene Oxide Adducts], Wiss. Verlagsgesellschaft, Stuttgart 1976; Winnacker-Küchler, “Chemische Technologie” [Chemical Engineering], volume 7, C. Hanser Verlag Munich, 4th Ed. 1986.

On the basis of these formulations, it is also possible to produce combinations with other pesticidally active substances, for example insecticides, acaricides, herbicides, fungicides, and also with safeners, fertilizers and/or growth regulators, for example in the form of a finished formulation or as a tankmix.

Wettable powders are preparations which can be dispersed uniformly in water and, in addition to the active ingredient, apart from a diluent or inert substance, also comprise surfactants of the ionic and/or nonionic type (wetting agents, dispersants), for example polyethoxylated alkylphenols, polyethoxylated fatty alcohols, polyethoxylated fatty amines, fatty alcohol polyglycol ether sulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurate. To produce the wettable powders, the herbicidally active ingredients are finely ground, for example in customary apparatus such as hammer mills, blower mills and air-jet mills, and simultaneously or subsequently mixed with the formulation auxiliaries.

Emulsifiable concentrates are produced by dissolving the active ingredient in an organic solvent, for example butanol, cyclohexanone, dimethylformamide, xylene, or else relatively high-boiling aromatics or hydrocarbons or mixtures of the organic solvents, with addition of one or more ionic and/or nonionic surfactants (emulsifiers). Examples of emulsifiers which may be used are: calcium alkylarylsulphonates such as calcium dodecylbenzenesulphonate, or nonionic emulsifiers such as fatty acid polyglycol esters, alkylaryl polyglycol ethers, fatty alcohol polyglycol ethers, propylene oxide-ethylene oxide condensation products, alkyl polyethers, sorbitan esters, for example sorbitan fatty acid esters, or polyoxyethylene sorbitan esters, for example polyoxyethylene sorbitan fatty acid esters.

Dustable powders are obtained by grinding the active ingredient with finely distributed solid substances, for example talc, natural clays such as kaolin, bentonite and pyrophyllite, or diatomaceous earth.

Suspension concentrates may be water- or oil-based. They may be prepared, for example, by wet-grinding by means of commercial bead mills and optional addition of surfactants as have, for example, already been listed above for the other formulation types.

Emulsions, for example oil-in-water emulsions (EW), can be produced, for example, by means of stirrers, colloid mills and/or static mixers using aqueous organic solvents and optionally surfactants as already listed above, for example, for the other formulation types.

Granules can be prepared either by spraying the active ingredient onto adsorptive granular inert material or by applying active ingredient concentrates to the surface of carriers, such as sand, kaolinites or granular inert material, by means of adhesives, for example polyvinyl alcohol, sodium polyacrylate or else mineral oils. Suitable active ingredients can also be granulated in the manner customary for the production of fertilizer granules—if desired as a mixture with fertilizers.

Water-dispersible granules are produced generally by the customary processes such as spray-drying, fluidized bed granulation, pan granulation, mixing with high-speed mixers and extrusion without solid inert material.

For the production of pan, fluidized-bed, extruder and spray granules, see e.g. processes in “Spray Drying Handbook” 3rd Ed. 1979, G. Goodwin Ltd., London, J. E. Browning, “Agglomeration”, Chemical and Engineering 1967, pages 147 ff.; “Perry's Chemical Engineer's Handbook”, 5th ed., McGraw-Hill, New York 1973, pp. 8-57.

For further details regarding the formulation of crop protection compositions, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

The agrochemical preparations contain generally 0.1 to 99% by weight, especially 0.1 to 95% by weight, of compounds of the invention.

In wettable powders, the active ingredient concentration is, for example, about 10% to 90% by weight, the remainder to 100% by weight consisting of customary formulation constituents. In emulsifiable concentrates, the active ingredient concentration may be about 1% to 90% and preferably 5% to 80% by weight. Dust-type formulations contain 1% to 30% by weight of active ingredient, preferably usually 5% to 20% by weight of active ingredient; sprayable solutions contain about 0.05% to 80% by weight, preferably 2% to 50% by weight of active ingredient. In the case of water-dispersible granules, the active ingredient content depends partially on whether the active compound is present in liquid or solid form and on which granulation auxiliaries, fillers, etc., are used. In the water-dispersible granules, the content of active ingredient is, for example, between 1% and 95% by weight, preferably between 10% and 80% by weight.

In addition, the active ingredient formulations mentioned optionally comprise the respective customary stickers, wetters, dispersants, emulsifiers, penetrants, preservatives, antifreeze agents and solvents, fillers, carriers and dyes, defoamers, evaporation inhibitors and agents which influence the pH and the viscosity.

For application, the formulations in commercial form are, if appropriate, diluted in a customary manner, for example in the case of wettable powders, emulsifiable concentrates, dispersions and water-dispersible granules with water. Dust-type preparations, granules for soil application or granules for scattering and sprayable solutions are not normally diluted further with other inert substances prior to application.

The required application rate of the compounds of the formula (I) varies with the external conditions, including temperature, humidity and the type of herbicide used. It can vary within wide limits, for example between 0.001 and 1.0 kg/ha or more of active substance, but it is preferably between 0.005 and 750 g/ha.

The examples listed in the table below are very particularly preferred.

The abbreviations used mean:

Me=methyl c-Pr=cyclopropyl

TABLE 1
Compounds according to the invention of the general formula (I) in
which R² and R³ each represent hydrogen and A represents a direct
bond, X¹ represents CH, X² represents CR⁷ and X³ represents CR⁸

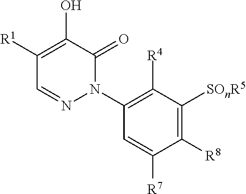

| No. | R¹ | R⁴ | n | R⁵ | R⁷ | R⁸ |
|---|---|---|---|---|---|---|
| 1-1 | Me | Me | 0 | c-Pr | H | H |
| 1-2 | Me | Me | 1 | c-Pr | H | H |
| 1-3 | Me | Me | 2 | c-Pr | H | H |
| 1-4 | Me | Cl | 0 | c-Pr | H | H |
| 1-5 | Me | Cl | 1 | c-Pr | H | H |
| 1-6 | Me | Cl | 2 | c-Pr | H | H |
| 1-7 | Me | Me | 0 | c-Pr | H | Me |
| 1-8 | Me | Me | 1 | c-Pr | H | Me |
| 1-9 | Me | Me | 2 | c-Pr | H | Me |
| 1-10 | Me | Cl | 0 | c-Pr | H | Me |
| 1-11 | Me | Cl | 1 | c-Pr | H | Me |
| 1-12 | Me | Cl | 2 | c-Pr | H | Me |
| 1-13 | Me | Me | 0 | c-Pr | H | Cl |
| 1-14 | Me | Me | 1 | c-Pr | H | Cl |
| 1-15 | Me | Me | 2 | c-Pr | H | Cl |
| 1-16 | Me | Cl | 0 | c-Pr | H | Cl |
| 1-17 | Me | Cl | 1 | c-Pr | H | Cl |
| 1-18 | Me | Cl | 2 | c-Pr | H | Cl |
| 1-19 | Me | Me | 0 | c-Pr | H | CF₃ |
| 1-20 | Me | Me | 1 | c-Pr | H | CF₃ |
| 1-21 | Me | Me | 2 | c-Pr | H | CF₃ |
| 1-22 | Me | Cl | 0 | c-Pr | H | CF₃ |
| 1-23 | Me | Cl | 1 | c-Pr | H | CF₃ |
| 1-24 | Me | Cl | 2 | c-Pr | H | CF₃ |
| 1-25 | Me | Me | 0 | c-Pr | H | SO₂Me |
| 1-26 | Me | Me | 1 | c-Pr | H | SO₂Me |
| 1-27 | Me | Me | 2 | c-Pr | H | SO₂Me |
| 1-28 | Me | Cl | 0 | c-Pr | H | SO₂Me |
| 1-29 | Me | Cl | 1 | c-Pr | H | SO₂Me |
| 1-30 | Me | Cl | 2 | c-Pr | H | SO₂Me |
| 1-31 | Me | Me | 0 | c-Pr | Me | H |
| 1-32 | Me | Me | 1 | c-Pr | Me | H |
| 1-33 | Me | Me | 2 | c-Pr | Me | H |
| 1-34 | Me | Cl | 0 | c-Pr | Me | H |
| 1-35 | Me | Cl | 1 | c-Pr | Me | H |
| 1-36 | Me | Cl | 2 | c-Pr | Me | H |
| 1-37 | Me | Me | 0 | c-Pr | Me | Me |
| 1-38 | Me | Me | 1 | c-Pr | Me | Me |
| 1-39 | Me | Me | 2 | c-Pr | Me | Me |
| 1-40 | Me | Cl | 0 | c-Pr | Me | Me |
| 1-41 | Me | Cl | 1 | c-Pr | Me | Me |
| 1-42 | Me | Cl | 2 | c-Pr | Me | Me |
| 1-43 | Me | Me | 0 | c-Pr | Me | Cl |
| 1-44 | Me | Me | 1 | c-Pr | Me | Cl |
| 1-45 | Me | Me | 2 | c-Pr | Me | Cl |
| 1-46 | Me | Cl | 0 | c-Pr | Me | Cl |
| 1-47 | Me | Cl | 1 | c-Pr | Me | Cl |
| 1-48 | Me | Cl | 2 | c-Pr | Me | Cl |
| 1-49 | Me | Me | 0 | c-Pr | Me | CF₃ |
| 1-50 | Me | Me | 1 | c-Pr | Me | CF₃ |
| 1-51 | Me | Me | 2 | c-Pr | Me | CF₃ |
| 1-52 | Me | Cl | 0 | c-Pr | Me | CF₃ |
| 1-53 | Me | Cl | 1 | c-Pr | Me | CF₃ |
| 1-54 | Me | Cl | 2 | c-Pr | Me | CF₃ |
| 1-55 | Me | Me | 0 | c-Pr | Me | SO₂Me |
| 1-56 | Me | Me | 1 | c-Pr | Me | SO₂Me |
| 1-57 | Me | Me | 2 | c-Pr | Me | SO₂Me |
| 1-58 | Me | Cl | 0 | c-Pr | Me | SO₂Me |
| 1-59 | Me | Cl | 1 | c-Pr | Me | SO₂Me |
| 1-60 | Me | Cl | 2 | c-Pr | Me | SO₂Me |
| 1-61 | c-Pr | Me | 0 | c-Pr | H | H |
| 1-62 | c-Pr | Me | 1 | c-Pr | H | H |
| 1-63 | c-Pr | Me | 2 | c-Pr | H | H |
| 1-64 | c-Pr | Cl | 0 | c-Pr | H | H |
| 1-65 | c-Pr | Cl | 1 | c-Pr | H | H |
| 1-66 | c-Pr | Cl | 2 | c-Pr | H | H |
| 1-67 | c-Pr | Me | 0 | c-Pr | H | Me |
| 1-68 | c-Pr | Me | 1 | c-Pr | H | Me |
| 1-69 | c-Pr | Me | 2 | c-Pr | H | Me |
| 1-70 | c-Pr | Cl | 0 | c-Pr | H | Me |
| 1-71 | c-Pr | Cl | 1 | c-Pr | H | Me |
| 1-72 | c-Pr | Cl | 2 | c-Pr | H | Me |
| 1-73 | c-Pr | Me | 0 | c-Pr | H | Cl |
| 1-74 | c-Pr | Me | 1 | c-Pr | H | Cl |
| 1-75 | c-Pr | Me | 2 | c-Pr | H | Cl |
| 1-76 | c-Pr | Cl | 0 | c-Pr | H | Cl |
| 1-77 | c-Pr | Cl | 1 | c-Pr | H | Cl |
| 1-78 | c-Pr | Cl | 2 | c-Pr | H | Cl |
| 1-79 | c-Pr | Me | 0 | c-Pr | H | CF₃ |
| 1-80 | c-Pr | Me | 1 | c-Pr | H | CF₃ |
| 1-81 | c-Pr | Me | 2 | c-Pr | H | CF₃ |
| 1-82 | c-Pr | Cl | 0 | c-Pr | H | CF₃ |
| 1-83 | c-Pr | Cl | 1 | c-Pr | H | CF₃ |
| 1-84 | c-Pr | Cl | 2 | c-Pr | H | CF₃ |
| 1-85 | c-Pr | Me | 0 | c-Pr | H | SO₂Me |
| 1-86 | c-Pr | Me | 1 | c-Pr | H | SO₂Me |
| 1-87 | c-Pr | Me | 2 | c-Pr | H | SO₂Me |
| 1-88 | c-Pr | Cl | 0 | c-Pr | H | SO₂Me |
| 1-89 | c-Pr | Cl | 1 | c-Pr | H | SO₂Me |
| 1-90 | c-Pr | Cl | 2 | c-Pr | H | SO₂Me |
| 1-91 | c-Pr | Me | 0 | c-Pr | Me | H |
| 1-92 | c-Pr | Me | 1 | c-Pr | Me | H |
| 1-93 | c-Pr | Me | 2 | c-Pr | Me | H |
| 1-94 | c-Pr | Cl | 0 | c-Pr | Me | H |
| 1-95 | c-Pr | Cl | 1 | c-Pr | Me | H |
| 1-96 | c-Pr | Cl | 2 | c-Pr | Me | H |
| 1-97 | c-Pr | Me | 0 | c-Pr | Me | Me |
| 1-98 | c-Pr | Me | 1 | c-Pr | Me | Me |
| 1-99 | c-Pr | Me | 2 | c-Pr | Me | Me |
| 1-100 | c-Pr | Cl | 0 | c-Pr | Me | Me |
| 1-101 | c-Pr | Cl | 1 | c-Pr | Me | Me |
| 1-102 | c-Pr | Cl | 2 | c-Pr | Me | Me |
| 1-103 | c-Pr | Me | 0 | c-Pr | Me | Cl |
| 1-104 | c-Pr | Me | 1 | c-Pr | Me | Cl |
| 1-105 | c-Pr | Me | 2 | c-Pr | Me | Cl |
| 1-106 | c-Pr | Cl | 0 | c-Pr | Me | Cl |
| 1-107 | c-Pr | Cl | 1 | c-Pr | Me | Cl |
| 1-108 | c-Pr | Cl | 2 | c-Pr | Me | Cl |
| 1-109 | c-Pr | Me | 0 | c-Pr | Me | CF₃ |
| 1-110 | c-Pr | Me | 1 | c-Pr | Me | CF₃ |
| 1-111 | c-Pr | Me | 2 | c-Pr | Me | CF₃ |
| 1-112 | c-Pr | Cl | 0 | c-Pr | Me | CF₃ |
| 1-113 | c-Pr | Cl | 1 | c-Pr | Me | CF₃ |
| 1-114 | c-Pr | Cl | 2 | c-Pr | Me | CF₃ |
| 1-115 | c-Pr | Me | 0 | c-Pr | Me | SO₂Me |
| 1-116 | c-Pr | Me | 1 | c-Pr | Me | SO₂Me |
| 1-117 | c-Pr | Me | 2 | c-Pr | Me | SO₂Me |
| 1-118 | c-Pr | Cl | 0 | c-Pr | Me | SO₂Me |
| 1-119 | c-Pr | Cl | 1 | c-Pr | Me | SO₂Me |
| 1-120 | c-Pr | Cl | 2 | c-Pr | Me | SO₂Me |
| 1-121 | Propen-2-yl | Me | 0 | c-Pr | H | H |
| 1-122 | Propen-2-yl | Me | 1 | c-Pr | H | H |
| 1-123 | Propen-2-yl | Me | 2 | c-Pr | H | H |
| 1-124 | Propen-2-yl | Cl | 0 | c-Pr | H | H |
| 1-125 | Propen-2-yl | Cl | 1 | c-Pr | H | H |
| 1-126 | Propen-2-yl | Cl | 2 | c-Pr | H | H |
| 1-127 | Propen-2-yl | Me | 0 | c-Pr | H | Me |
| 1-128 | Propen-2-yl | Me | 1 | c-Pr | H | Me |
| 1-129 | Propen-2-yl | Me | 2 | c-Pr | H | Me |
| 1-130 | Propen-2-yl | Cl | 0 | c-Pr | H | Me |
| 1-131 | Propen-2-yl | Cl | 1 | c-Pr | H | Me |
| 1-132 | Propen-2-yl | Cl | 2 | c-Pr | H | Me |
| 1-133 | Propen-2-yl | Me | 0 | c-Pr | H | Cl |
| 1-134 | Propen-2-yl | Me | 1 | c-Pr | H | Cl |
| 1-135 | Propen-2-yl | Me | 2 | c-Pr | H | Cl |
| 1-136 | Propen-2-yl | Cl | 0 | c-Pr | H | Cl |
| 1-137 | Propen-2-yl | Cl | 1 | c-Pr | H | Cl |
| 1-138 | Propen-2-yl | Cl | 2 | c-Pr | H | Cl |
| 1-139 | Propen-2-yl | Me | 0 | c-Pr | H | CF₃ |
| 1-140 | Propen-2-yl | Me | 1 | c-Pr | H | CF₃ |
| 1-141 | Propen-2-yl | Me | 2 | c-Pr | H | CF₃ |
| 1-142 | Propen-2-yl | Cl | 0 | c-Pr | H | CF₃ |
| 1-143 | Propen-2-yl | Cl | 1 | c-Pr | H | CF₃ |
| 1-144 | Propen-2-yl | Cl | 2 | c-Pr | H | CF₃ |
| 1-145 | Propen-2-yl | Me | 0 | c-Pr | H | SO₂Me |
| 1-146 | Propen-2-yl | Me | 1 | c-Pr | H | SO₂Me |
| 1-147 | Propen-2-yl | Me | 2 | c-Pr | H | SO₂Me |
| 1-148 | Propen-2-yl | Cl | 0 | c-Pr | H | SO₂Me |
| 1-149 | Propen-2-yl | Cl | 1 | c-Pr | H | SO₂Me |
| 1-150 | Propen-2-yl | Cl | 2 | c-Pr | H | SO₂Me |
| 1-151 | Propen-2-yl | Me | 0 | c-Pr | Me | H |
| 1-152 | Propen-2-yl | Me | 1 | c-Pr | Me | H |
| 1-153 | Propen-2-yl | Me | 2 | c-Pr | Me | H |
| 1-154 | Propen-2-yl | Cl | 0 | c-Pr | Me | H |
| 1-155 | Propen-2-yl | Cl | 1 | c-Pr | Me | H |
| 1-156 | Propen-2-yl | Cl | 2 | c-Pr | Me | H |
| 1-157 | Propen-2-yl | Me | 0 | c-Pr | Me | Me |
| 1-158 | Propen-2-yl | Me | 1 | c-Pr | Me | Me |
| 1-159 | Propen-2-yl | Me | 2 | c-Pr | Me | Me |
| 1-160 | Propen-2-yl | Cl | 0 | c-Pr | Me | Me |
| 1-161 | Propen-2-yl | Cl | 1 | c-Pr | Me | Me |
| 1-162 | Propen-2-yl | Cl | 2 | c-Pr | Me | Me |
| 1-163 | Propen-2-yl | Me | 0 | c-Pr | Me | Cl |
| 1-164 | Propen-2-yl | Me | 1 | c-Pr | Me | Cl |
| 1-165 | Propen-2-yl | Me | 2 | c-Pr | Me | Cl |
| 1-166 | Propen-2-yl | Cl | 0 | c-Pr | Me | Cl |
| 1-167 | Propen-2-yl | Cl | 1 | c-Pr | Me | Cl |
| 1-168 | Propen-2-yl | Cl | 2 | c-Pr | Me | Cl |
| 1-169 | Propen-2-yl | Me | 0 | c-Pr | Me | CF₃ |
| 1-170 | Propen-2-yl | Me | 1 | c-Pr | Me | CF₃ |
| 1-171 | Propen-2-yl | Me | 2 | c-Pr | Me | CF₃ |
| 1-172 | Propen-2-yl | Cl | 0 | c-Pr | Me | CF₃ |
| 1-173 | Propen-2-yl | Cl | 1 | c-Pr | Me | CF₃ |
| 1-174 | Propen-2-yl | Cl | 2 | c-Pr | Me | CF₃ |
| 1-175 | Propen-2-yl | Me | 0 | c-Pr | Me | SO₂Me |
| 1-176 | Propen-2-yl | Me | 1 | c-Pr | Me | SO₂Me |
| 1-177 | Propen-2-yl | Me | 2 | c-Pr | Me | SO₂Me |
| 1-178 | Propen-2-yl | Cl | 0 | c-Pr | Me | SO₂Me |
| 1-179 | Propen-2-yl | Cl | 1 | c-Pr | Me | SO₂Me |
| 1-180 | Propen-2-yl | Cl | 2 | c-Pr | Me | SO₂Me |
| 1-181 | Cl | Me | 0 | c-Pr | H | H |
| 1-182 | Cl | Me | 1 | c-Pr | H | H |
| 1-183 | Cl | Me | 2 | c-Pr | H | H |
| 1-184 | Cl | Cl | 0 | c-Pr | H | H |
| 1-185 | Cl | Cl | 1 | c-Pr | H | H |
| 1-186 | Cl | Cl | 2 | c-Pr | H | H |
| 1-187 | Cl | Me | 0 | c-Pr | H | Me |
| 1-188 | Cl | Me | 1 | c-Pr | H | Me |
| 1-189 | Cl | Me | 2 | c-Pr | H | Me |
| 1-190 | Cl | Cl | 0 | c-Pr | H | Me |
| 1-191 | Cl | Cl | 1 | c-Pr | H | Me |
| 1-192 | Cl | Cl | 2 | c-Pr | H | Me |
| 1-193 | Cl | Me | 0 | c-Pr | H | Cl |
| 1-194 | Cl | Me | 1 | c-Pr | H | Cl |
| 1-195 | Cl | Me | 2 | c-Pr | H | Cl |
| 1-196 | Cl | Cl | 0 | c-Pr | I-I | Cl |
| 1-197 | Cl | Cl | 1 | c-Pr | H | Cl |
| 1-198 | Cl | Cl | 2 | c-Pr | H | Cl |
| 1-199 | Cl | Me | 0 | c-Pr | H | CF₃ |
| 1-200 | Cl | Me | 1 | c-Pr | H | CF₃ |
| 1-201 | Cl | Me | 2 | c-Pr | H | CF₃ |
| 1-202 | Cl | Cl | 0 | c-Pr | H | CF₃ |
| 1-203 | Cl | Cl | 1 | c-Pr | H | CF₃ |
| 1-204 | Cl | Cl | 2 | c-Pr | H | CF₃ |
| 1-205 | Cl | Me | 0 | c-Pr | H | SO₂Me |
| 1-206 | Cl | Me | 1 | c-Pr | H | SO₂Me |
| 1-207 | Cl | Me | 2 | c-Pr | H | SO₂Me |
| 1-208 | Cl | Cl | 0 | c-Pr | H | SO₂Me |
| 1-209 | Cl | Cl | 1 | c-Pr | H | SO₂Me |
| 1-210 | Cl | Cl | 2 | c-Pr | H | SO₂Me |
| 1-211 | Cl | Me | 0 | c-Pr | Me | H |
| 1-212 | Cl | Me | 1 | c-Pr | Me | H |
| 1-213 | Cl | Me | 2 | c-Pr | Me | H |
| 1-214 | Cl | Cl | 0 | c-Pr | Me | H |
| 1-215 | Cl | Cl | 1 | c-Pr | Me | H |
| 1-216 | Cl | Cl | 2 | c-Pr | Me | H |
| 1-217 | Cl | Me | 0 | c-Pr | Me | Me |
| 1-218 | Cl | Me | 1 | c-Pr | Me | Me |
| 1-219 | Cl | Me | 2 | c-Pr | Me | Me |
| 1-220 | Cl | Cl | 0 | c-Pr | Me | Me |
| 1-221 | Cl | Cl | 1 | c-Pr | Me | Me |
| 1-222 | Cl | Cl | 2 | c-Pr | Me | Me |
| 1-223 | Cl | Me | 0 | c-Pr | Me | Cl |
| 1-224 | Cl | Me | 1 | c-Pr | Me | Cl |
| 1-225 | Cl | Me | 2 | c-Pr | Me | Cl |
| 1-226 | Cl | Cl | 0 | c-Pr | Me | Cl |
| 1-227 | Cl | Cl | 1 | c-Pr | Me | Cl |
| 1-228 | Cl | Cl | 2 | c-Pr | Me | Cl |
| 1-229 | Cl | Me | 0 | c-Pr | Me | CF₃ |
| 1-230 | Cl | Me | 1 | c-Pr | Me | CF₃ |
| 1-231 | Cl | Me | 2 | c-Pr | Me | CF₃ |
| 1-232 | Cl | Cl | 0 | c-Pr | Me | CF₃ |
| 1-233 | Cl | Cl | 1 | c-Pr | Me | CF₃ |
| 1-234 | Cl | Cl | 2 | c-Pr | Me | CF₃ |
| 1-235 | Cl | Me | 0 | c-Pr | Me | SO₂Me |
| 1-236 | Cl | Me | 1 | c-Pr | Me | SO₂Me |
| 1-237 | Cl | Me | 2 | c-Pr | Me | SO₂Me |
| 1-238 | Cl | Cl | 0 | c-Pr | Me | SO₂Me |
| 1-239 | Cl | Cl | 1 | c-Pr | Me | SO₂Me |
| 1-240 | Cl | Cl | 2 | c-Pr | Me | SO₂Me |
| 1-241 | SO₂Me | Me | 0 | c-Pr | H | H |
| 1-242 | SO₂Me | Me | 1 | c-Pr | H | H |
| 1-243 | SO₂Me | Me | 2 | c-Pr | H | H |
| 1-244 | SO₂Me | Cl | 0 | c-Pr | H | H |
| 1-245 | SO₂Me | Cl | 1 | c-Pr | H | H |
| 1-246 | SO₂Me | Cl | 2 | c-Pr | H | H |
| 1-247 | SO₂Me | Me | 0 | c-Pr | H | Me |
| 1-248 | SO₂Me | Me | 1 | c-Pr | H | Me |
| 1-249 | SO₂Me | Me | 2 | c-Pr | H | Me |
| 1-250 | SO₂Me | Cl | 0 | c-Pr | H | Me |
| 1-251 | SO₂Me | Cl | 1 | c-Pr | H | Me |
| 1-252 | SO₂Me | Cl | 2 | c-Pr | H | Me |
| 1-253 | SO₂Me | Me | 0 | c-Pr | H | Cl |
| 1-254 | SO₂Me | Me | 1 | c-Pr | H | Cl |
| 1-255 | SO₂Me | Me | 2 | c-Pr | H | Cl |
| 1-256 | SO₂Me | Cl | 0 | c-Pr | H | Cl |
| 1-257 | SO₂Me | Cl | 1 | c-Pr | H | Cl |
| 1-258 | SO₂Me | Cl | 2 | c-Pr | H | Cl |
| 1-259 | SO₂Me | Me | 0 | c-Pr | H | CF₃ |
| 1-260 | SO₂Me | Me | 1 | c-Pr | H | CF₃ |
| 1-261 | SO₂Me | Me | 2 | c-Pr | H | CF₃ |
| 1-262 | SO₂Me | Cl | 0 | c-Pr | H | CF₃ |
| 1-263 | SO₂Me | Cl | 1 | c-Pr | H | CF₃ |
| 1-264 | SO₂Me | Cl | 2 | c-Pr | H | CF₃ |
| 1-265 | SO₂Me | Me | 0 | c-Pr | H | SO₂Me |
| 1-266 | SO₂Me | Me | 1 | c-Pr | H | SO₂Me |
| 1-267 | SO₂Me | Me | 2 | c-Pr | H | SO₂Me |
| 1-268 | SO₂Me | Cl | 0 | c-Pr | H | SO₂Me |
| 1-269 | SO₂Me | Cl | 1 | c-Pr | H | SO₂Me |
| 1-270 | SO₂Me | Cl | 2 | c-Pr | H | SO₂Me |
| 1-271 | SO₂Me | Me | 0 | c-Pr | Me | H |
| 1-272 | SO₂Me | Me | 1 | c-Pr | Me | H |
| 1-273 | SO₂Me | Me | 2 | c-Pr | Me | H |
| 1-274 | SO₂Me | Cl | 0 | c-Pr | Me | H |
| 1-275 | SO₂Me | Cl | 1 | c-Pr | Me | H |
| 1-276 | SO₂Me | Cl | 2 | c-Pr | Me | H |
| 1-277 | SO₂Me | Me | 0 | c-Pr | Me | Me |
| 1-278 | SO₂Me | Me | 1 | c-Pr | Me | Me |
| 1-279 | SO₂Me | Me | 2 | c-Pr | Me | Me |
| 1-280 | SO₂Me | Cl | 0 | c-Pr | Me | Me |
| 1-281 | SO₂Me | Cl | 1 | c-Pr | Me | Me |
| 1-282 | SO₂Me | Cl | 2 | c-Pr | Me | Me |
| 1-283 | SO₂Me | Me | 0 | c-Pr | Me | Cl |
| 1-284 | SO₂Me | Me | 1 | c-Pr | Me | Cl |
| 1-285 | SO₂Me | Me | 2 | c-Pr | Me | Cl |
| 1-286 | SO₂Me | Cl | 0 | c-Pr | Me | Cl |
| 1-287 | SO₂Me | Cl | 1 | c-Pr | Me | Cl |
| 1-288 | SO₂Me | Cl | 2 | c-Pr | Me | Cl |
| 1-289 | SO₂Me | Me | 0 | c-Pr | Me | CF₃ |
| 1-290 | SO₂Me | Me | 1 | c-Pr | Me | CF₃ |
| 1-291 | SO₂Me | Me | 2 | c-Pr | Me | CF₃ |
| 1-292 | SO₂Me | Cl | 0 | c-Pr | Me | CF₃ |
| 1-293 | SO₂Me | Cl | 1 | c-Pr | Me | CF₃ |
| 1-294 | SO₂Me | Cl | 2 | c-Pr | Me | CF₃ |
| 1-295 | SO₂Me | Me | 0 | c-Pr | Me | SO₂Me |
| 1-296 | SO₂Me | Me | 1 | c-Pr | Me | SO₂Me |
| 1-297 | SO₂Me | Me | 2 | c-Pr | Me | SO₂Me |
| 1-298 | SO₂Me | Cl | 0 | c-Pr | Me | SO₂Me |
| 1-299 | SO₂Me | Cl | 1 | c-Pr | Me | SO₂Me |
| 1-300 | SO₂Me | Cl | 2 | c-Pr | Me | SO₂Me |
| 1-301 | Me | Me | 0 | CH₂-c-Pr | H | H |
| 1-302 | Me | Me | 1 | CH₂-c-Pr | H | H |
| 1-303 | Me | Me | 2 | CH₂-c-Pr | H | H |
| 1-304 | Me | Cl | 0 | CH₂-c-Pr | H | H |
| 1-305 | Me | Cl | 1 | CH₂-c-Pr | H | H |
| 1-306 | Me | Cl | 2 | CH₂-c-Pr | H | H |
| 1-307 | Me | Me | 0 | CH₂-c-Pr | H | Me |
| 1-308 | Me | Me | 1 | CH₂-c-Pr | H | Me |
| 1-309 | Me | Me | 2 | CH₂-c-Pr | H | Me |
| 1-310 | Me | Cl | 0 | CH₂-c-Pr | H | Me |
| 1-311 | Me | Cl | 1 | CH₂-c-Pr | H | Me |
| 1-312 | Me | Cl | 2 | CH₂-c-Pr | H | Me |
| 1-313 | Me | Me | 0 | CH₂-c-Pr | H | Cl |
| 1-314 | Me | Me | 1 | CH₂-c-Pr | H | Cl |
| 1-315 | Me | Me | 2 | CH₂-c-Pr | H | Cl |
| 1-316 | Me | Cl | 0 | CH₂-c-Pr | H | Cl |
| 1-317 | Me | Cl | 1 | CH₂-c-Pr | H | Cl |
| 1-318 | Me | Cl | 2 | CH₂-c-Pr | H | Cl |
| 1-319 | Me | Me | 0 | CH₂-c-Pr | H | CF₃ |
| 1-320 | Me | Me | 1 | CH₂-c-Pr | H | CF₃ |
| 1-321 | Me | Me | 2 | CH₂-c-Pr | H | CF₃ |
| 1-322 | Me | Cl | 0 | CH₂-c-Pr | H | CF₃ |
| 1-323 | Me | Cl | 1 | CH₂-c-Pr | H | CF₃ |
| 1-324 | Me | Cl | 2 | CH₂-c-Pr | H | CF₃ |
| 1-325 | Me | Me | 0 | CH₂-c-Pr | H | SO₂Me |
| 1-326 | Me | Me | 1 | CH₂-c-Pr | H | SO₂Me |
| 1-327 | Me | Me | 2 | CH₂-c-Pr | H | SO₂Me |
| 1-328 | Me | Cl | 0 | CH₂-c-Pr | H | SO₂Me |
| 1-329 | Me | Cl | 1 | CH₂-c-Pr | H | SO₂Me |
| 1-330 | Me | Cl | 2 | CH₂-c-Pr | H | SO₂Me |
| 1-331 | Me | Me | 0 | CH₂-c-Pr | Me | H |
| 1-332 | Me | Me | 1 | CH₂-c-Pr | Me | H |
| 1-333 | Me | Me | 2 | CH₂-c-Pr | Me | H |
| 1-334 | Me | Cl | 0 | CH₂-c-Pr | Me | H |
| 1-335 | Me | Cl | 1 | CH₂-c-Pr | Me | H |
| 1-336 | Me | Cl | 2 | CH₂-c-Pr | Me | H |
| 1-337 | Me | Me | 0 | CH₂-c-Pr | Me | Me |
| 1-338 | Me | Me | 1 | CH₂-c-Pr | Me | Me |
| 1-339 | Me | Me | 2 | CH₂-c-Pr | Me | Me |
| 1-340 | Me | Cl | 0 | CH2-c-Pr | Me | Me |
| 1-341 | Me | Cl | 1 | CH2-c-Pr | Me | Me |
| 1-342 | Me | Cl | 2 | CH₂-c-Pr | Me | Me |
| 1-343 | Me | Me | 0 | CH₂-c-Pr | Me | Cl |
| 1-344 | Me | Me | 1 | CH₂-c-Pr | Me | Cl |
| 1-345 | Me | Me | 2 | CH₂-c-Pr | Me | Cl |
| 1-346 | Me | Cl | 0 | CH₂-c-Pr | Me | Cl |
| 1-347 | Me | Cl | 1 | CH₂-c-Pr | Me | Cl |
| 1-348 | Me | Cl | 2 | CH₂-c-Pr | Me | Cl |
| 1-349 | Me | Me | 0 | CH₂-c-Pr | Me | CF₃ |
| 1-350 | Me | Me | 1 | CH₂-c-Pr | Me | CF₃ |
| 1-351 | Me | Me | 2 | CH₂-c-Pr | Me | CF₃ |
| 1-352 | Me | Cl | 0 | CH₂-c-Pr | Me | CF₃ |
| 1-353 | Me | Cl | 1 | CH₂-c-Pr | Me | CF₃ |
| 1-354 | Me | Cl | 2 | CH₂-c-Pr | Me | CF₃ |
| 1-355 | Me | Me | 0 | CH₂-c-Pr | Me | SO₂Me |
| 1-356 | Me | Me | 1 | CH₂-c-Pr | Me | SO₂Me |
| 1-357 | Me | Me | 2 | CH₂-c-Pr | Me | SO₂Me |
| 1-358 | Me | Cl | 0 | CH₂-c-Pr | Me | SO₂Me |
| 1-359 | Me | Cl | 1 | CH₂-c-Pr | Me | SO₂Me |
| 1-360 | Me | Cl | 2 | CH₂-c-Pr | Me | SO₂Me |
| 1-361 | c-Pr | Me | 0 | CH₂-c-Pr | H | H |
| 1-362 | c-Pr | Me | 1 | CH₂-c-Pr | H | H |
| 1-363 | c-Pr | Me | 2 | CH₂-c-Pr | H | H |
| 1-364 | c-Pr | Cl | 0 | CH₂-c-Pr | H | H |
| 1-365 | c-Pr | Cl | 1 | CH₂-c-Pr | H | H |
| 1-366 | c-Pr | Cl | 2 | CH₂-c-Pr | H | H |
| 1-367 | c-Pr | Me | 0 | CH₂-c-Pr | H | Me |
| 1-368 | c-Pr | Me | 1 | CH₂-c-Pr | H | Me |
| 1-369 | c-Pr | Me | 2 | CH₂-c-Pr | H | Me |
| 1-370 | c-Pr | Cl | 0 | CH₂-c-Pr | H | Me |
| 1-371 | c-Pr | Cl | 1 | CH₂-c-Pr | H | Me |
| 1-372 | c-Pr | Cl | 2 | CH₂-c-Pr | H | Me |
| 1-373 | c-Pr | Me | 0 | CH₂-c-Pr | H | Cl |
| 1-374 | c-Pr | Me | 1 | CH₂-c-Pr | H | Cl |
| 1-375 | c-Pr | Me | 2 | CH₂-c-Pr | H | Cl |
| 1-376 | c-Pr | Cl | 0 | CH₂-c-Pr | H | Cl |
| 1-377 | c-Pr | Cl | 1 | CH₂-c-Pr | H | Cl |
| 1-378 | c-Pr | Cl | 2 | CH₂-c-Pr | H | Cl |
| 1-379 | c-Pr | Me | 0 | CH₂-c-Pr | H | CF₃ |
| 1-380 | c-Pr | Me | 1 | CH₂-c-Pr | H | CF₃ |
| 1-381 | c-Pr | Me | 2 | CH₂-c-Pr | H | CF₃ |
| 1-382 | c-Pr | Cl | 0 | CH₂-c-Pr | H | CF₃ |
| 1-383 | c-Pr | Cl | 1 | CH₂-c-Pr | H | CF₃ |
| 1-384 | c-Pr | Cl | 2 | CH₂-c-Pr | H | CF₃ |
| 1-385 | c-Pr | Me | 0 | CH₂-c-Pr | H | SO₂Me |
| 1-386 | c-Pr | Me | 1 | CH₂-c-Pr | H | SO₂Me |
| 1-387 | c-Pr | Me | 2 | CH₂-c-Pr | H | SO₂Me |
| 1-388 | c-Pr | Cl | 0 | CH₂-c-Pr | H | SO₂Me |
| 1-389 | c-Pr | Cl | 1 | CH₂-c-Pr | H | SO₂Me |
| 1-390 | c-Pr | Cl | 2 | CH₂-c-Pr | H | SO₂Me |
| 1-391 | c-Pr | Me | 0 | CH₂-c-Pr | Me | H |
| 1-392 | c-Pr | Me | 1 | CH₂-c-Pr | Me | H |
| 1-393 | c-Pr | Me | 2 | CH₂-c-Pr | Me | H |
| 1-394 | c-Pr | Cl | 0 | CH₂-c-Pr | Me | H |
| 1-395 | c-Pr | Cl | 1 | CH₂-c-Pr | Me | H |
| 1-396 | c-Pr | Cl | 2 | CH₂-c-Pr | Me | H |
| 1-397 | c-Pr | Me | 0 | CH₂-c-Pr | Me | Me |
| 1-398 | c-Pr | Me | 1 | CH₂-c-Pr | Me | Me |
| 1-399 | c-Pr | Me | 2 | CH₂-c-Pr | Me | Me |
| 1-400 | c-Pr | Cl | 0 | CH₂-c-Pr | Me | Me |
| 1-401 | c-Pr | Cl | 1 | CH₂-c-Pr | Me | Me |
| 1-402 | c-Pr | Cl | 2 | CH₂-c-Pr | Me | Me |
| 1-403 | c-Pr | Me | 0 | CH₂-c-Pr | Me | Cl |
| 1-404 | c-Pr | Me | 1 | CH₂-c-Pr | Me | Cl |
| 1-405 | c-Pr | Me | 2 | CH₂-c-Pr | Me | Cl |
| 1-406 | c-Pr | Cl | 0 | CH₂-c-Pr | Me | Cl |
| 1-407 | c-Pr | Cl | 1 | CH₂-c-Pr | Me | Cl |
| 1-408 | c-Pr | Cl | 2 | CH₂-c-Pr | Me | Cl |
| 1-409 | c-Pr | Me | 0 | CH₂-c-Pr | Me | CF₃ |
| 1-410 | c-Pr | Me | 1 | CH₂-c-Pr | Me | CF₃ |
| 1-411 | c-Pr | Me | 2 | CH₂-c-Pr | Me | CF₃ |
| 1-412 | c-Pr | Cl | 0 | CH₂-c-Pr | Me | CF₃ |
| 1-413 | c-Pr | Cl | 1 | CH₂-c-Pr | Me | CF₃ |
| 1-414 | c-Pr | Cl | 2 | CH₂-c-Pr | Me | CF₃ |
| 1-415 | c-Pr | Me | 0 | CH₂-c-Pr | Me | SO₂Me |
| 1-416 | c-Pr | Me | 1 | CH₂-c-Pr | Me | SO₂Me |
| 1-417 | c-Pr | Me | 2 | CH₂-c-Pr | Me | SO₂Me |
| 1-418 | c-Pr | Cl | 0 | CH₂-c-Pr | Me | SO₂Me |
| 1-419 | c-Pr | Cl | 1 | CH₂-c-Pr | Me | SO₂Me |
| 1-420 | c-Pr | Cl | 2 | CH₂-c-Pr | Me | SO₂Me |
| 1-421 | Propen-2-yl | Me | 0 | CH₂-c-Pr | H | H |
| 1-422 | Propen-2-yl | Me | 1 | CH₂-c-Pr | H | H |
| 1-423 | Propen-2-yl | Me | 2 | CH₂-c-Pr | H | H |
| 1-424 | Propen-2-yl | Cl | 0 | CH₂-c-Pr | H | H |
| 1-425 | Propen-2-yl | Cl | 1 | CH₂-c-Pr | H | H |
| 1-426 | Propen-2-yl | Cl | 2 | CH₂-c-Pr | H | H |
| 1-427 | Propen-2-yl | Me | 0 | CH₂-c-Pr | H | Me |
| 1-428 | Propen-2-yl | Me | 1 | CH₂-c-Pr | H | Me |
| 1-429 | Propen-2-yl | Me | 2 | CH₂-c-Pr | H | Me |
| 1-430 | Propen-2-yl | Cl | 0 | CH₂-c-Pr | H | Me |
| 1-431 | Propen-2-yl | Cl | 1 | CH₂-c-Pr | H | Me |
| 1-432 | Propen-2-yl | Cl | 2 | CH₂-c-Pr | H | Me |
| 1-433 | Propen-2-yl | Me | 0 | CH₂-c-Pr | H | Cl |
| 1-434 | Propen-2-yl | Me | 1 | CH₂-c-Pr | H | Cl |
| 1-435 | Propen-2-yl | Me | 2 | CH₂-c-Pr | H | Cl |
| 1-436 | Propen-2-yl | Cl | 0 | CH₂-c-Pr | H | Cl |
| 1-437 | Propen-2-yl | Cl | 1 | CH₂-c-Pr | H | Cl |
| 1-438 | Propen-2-yl | Cl | 2 | CH₂-c-Pr | H | Cl |
| 1-439 | Propen-2-yl | Me | 0 | CH₂-c-Pr | H | CF₃ |
| 1-440 | Propen-2-yl | Me | 1 | CH₂-c-Pr | H | CF₃ |
| 1-441 | Propen-2-yl | Me | 2 | CH₂-c-Pr | H | CF₃ |
| 1-442 | Propen-2-yl | Cl | 0 | CH₂-c-Pr | H | CF₃ |
| 1-443 | Propen-2-yl | Cl | 1 | CH₂-c-Pr | H | CF₃ |
| 1-444 | Propen-2-yl | Cl | 2 | CH₂-c-Pr | H | CF₃ |
| 1-445 | Propen-2-yl | Me | 0 | CH₂-c-Pr | H | SO₂Me |
| 1-446 | Propen-2-yl | Me | 1 | CH₂-c-Pr | H | SO₂Me |
| 1-447 | Propen-2-yl | Me | 2 | CH₂-c-Pr | H | SO₂Me |
| 1-448 | Propen-2-yl | Cl | 0 | CH₂-c-Pr | H | SO₂Me |
| 1-449 | Propen-2-yl | Cl | 1 | CH₂-c-Pr | H | SO₂Me |
| 1-450 | Propen-2-yl | Cl | 2 | CH₂-c-Pr | H | SO₂Me |
| 1-451 | Propen-2-yl | Me | 0 | CH₂-c-Pr | Me | H |
| 1-452 | Propen-2-yl | Me | 1 | CH₂-c-Pr | Me | H |
| 1-453 | Propen-2-yl | Me | 2 | CH₂-c-Pr | Me | H |
| 1-454 | Propen-2-yl | Cl | 0 | CH₂-c-Pr | Me | H |
| 1-455 | Propen-2-yl | Cl | 1 | CH₂-c-Pr | Me | H |
| 1-456 | Propen-2-yl | Cl | 2 | CH₂-c-Pr | Me | H |
| 1-457 | Propen-2-yl | Me | 0 | CH₂-c-Pr | Me | Me |
| 1-458 | Propen-2-yl | Me | 1 | CH₂-c-Pr | Me | Me |
| 1-459 | Propen-2-yl | Me | 2 | CH₂-c-Pr | Me | Me |
| 1-460 | Propen-2-yl | Cl | 0 | CH₂-c-Pr | Me | Me |
| 1-461 | Propen-2-yl | Cl | 1 | CH₂-c-Pr | Me | Me |
| 1-462 | Propen-2-yl | Cl | 2 | CH₂-c-Pr | Me | Me |
| 1-463 | Propen-2-yl | Me | 0 | CH₂-c-Pr | Me | Cl |
| 1-464 | Propen-2-yl | Me | 1 | CH₂-c-Pr | Me | Cl |
| 1-465 | Propen-2-yl | Me | 2 | CH₂-c-Pr | Me | Cl |
| 1-466 | Propen-2-yl | Cl | 0 | CH₂-c-Pr | Me | Cl |
| 1-467 | Propen-2-yl | Cl | 1 | CH₂-c-Pr | Me | Cl |
| 1-468 | Propen-2-yl | Cl | 2 | CH₂-c-Pr | Me | Cl |
| 1-469 | Propen-2-yl | Me | 0 | CH₂-c-Pr | Me | CF₃ |
| 1-470 | Propen-2-yl | Me | 1 | CH₂-c-Pr | Me | CF₃ |
| 1-471 | Propen-2-yl | Me | 2 | CH₂-c-Pr | Me | CF₃ |
| 1-472 | Propen-2-yl | Cl | 0 | CH₂-c-Pr | Me | CF₃ |
| 1-473 | Propen-2-yl | Cl | 1 | CH₂-c-Pr | Me | CF₃ |
| 1-474 | Propen-2-yl | Cl | 2 | CH₂-c-Pr | Me | CF₃ |
| 1-475 | Propen-2-yl | Me | 0 | CH₂-c-Pr | Me | SO₂Me |
| 1-476 | Propen-2-yl | Me | 1 | CH₂-c-Pr | Me | SO₂Me |
| 1-477 | Propen-2-yl | Me | 2 | CH₂-c-Pr | Me | SO₂Me |
| 1-478 | Propen-2-yl | Cl | 0 | CH₂-c-Pr | Me | SO₂Me |
| 1-479 | Propen-2-yl | Cl | 1 | CH₂-c-Pr | Me | SO₂Me |
| 1-480 | Propen-2-yl | Cl | 2 | CH₂-c-Pr | Me | SO₂Me |
| 1-481 | Cl | Me | 0 | CH₂-c-Pr | H | H |
| 1-482 | Cl | Me | 1 | CH₂-c-Pr | H | H |
| 1-483 | Cl | Me | 2 | CH₂-c-Pr | H | H |
| 1-484 | Cl | Cl | 0 | CH₂-c-Pr | H | H |
| 1-485 | Cl | Cl | 1 | CH₂-c-Pr | H | H |
| 1-486 | Cl | Cl | 2 | CH₂-c-Pr | H | H |
| 1-487 | Cl | Me | 0 | CH₂-c-Pr | H | Me |
| 1-488 | Cl | Me | 1 | CH₂-c-Pr | H | Me |
| 1-489 | Cl | Me | 2 | CH₂-c-Pr | H | Me |
| 1-490 | Cl | Cl | 0 | CH₂-c-Pr | H | Me |
| 1-491 | Cl | Cl | 1 | CH₂-c-Pr | H | Me |
| 1-492 | Cl | Cl | 2 | CH₂-c-Pr | H | Me |
| 1-493 | Cl | Me | 0 | CH₂-c-Pr | H | Cl |
| 1-494 | Cl | Me | 1 | CH₂-c-Pr | H | Cl |
| 1-495 | Cl | Me | 2 | CH₂-c-Pr | H | Cl |
| 1-496 | Cl | Cl | 0 | CH₂-c-Pr | H | Cl |
| 1-497 | Cl | Cl | 1 | CH₂-c-Pr | H | Cl |
| 1-498 | Cl | Cl | 2 | CH₂-c-Pr | H | Cl |
| 1-499 | Cl | Me | 0 | CH₂-c-Pr | H | CF₃ |
| 1-500 | Cl | Me | 1 | CH₂-c-Pr | H | CF₃ |
| 1-501 | Cl | Me | 2 | CH₂-c-Pr | H | CF₃ |
| 1-502 | Cl | Cl | 0 | CH₂-c-Pr | H | CF₃ |
| 1-503 | Cl | Cl | 1 | CH₂-c-Pr | H | CF₃ |
| 1-504 | Cl | Cl | 2 | CH₂-c-Pr | H | CF₃ |
| 1-505 | Cl | Me | 0 | CH₂-c-Pr | H | SO₂Me |
| 1-506 | Cl | Me | 1 | CH₂-c-Pr | H | SO₂Me |
| 1-507 | Cl | Me | 2 | CH₂-c-Pr | H | SO₂Me |
| 1-508 | Cl | Cl | 0 | CH₂-c-Pr | H | SO₂Me |
| 1-509 | Cl | Cl | 1 | CH₂-c-Pr | H | SO₂Me |
| 1-510 | Cl | Cl | 2 | CH₂-c-Pr | H | SO₂Me |
| 1-511 | Cl | Me | 0 | CH₂-c-Pr | Me | H |
| 1-512 | Cl | Me | 1 | CH₂-c-Pr | Me | H |
| 1-513 | Cl | Me | 2 | CH₂-c-Pr | Me | H |
| 1-514 | Cl | Cl | 0 | CH₂-c-Pr | Me | H |
| 1-515 | Cl | Cl | 1 | CH₂-c-Pr | Me | H |
| 1-516 | Cl | Cl | 2 | CH₂-c-Pr | Me | H |
| 1-517 | Cl | Me | 0 | CH₂-c-Pr | Me | Me |
| 1-518 | Cl | Me | 1 | CH₂-c-Pr | Me | Me |
| 1-519 | Cl | Me | 2 | CH₂-c-Pr | Me | Me |
| 1-520 | Cl | Cl | 0 | CH₂-c-Pr | Me | Me |
| 1-521 | Cl | Cl | 1 | CH₂-c-Pr | Me | Me |
| 1-522 | Cl | Cl | 2 | CH₂-c-Pr | Me | Me |
| 1-523 | Cl | Me | 0 | CH₂-c-Pr | Me | Cl |
| 1-524 | Cl | Me | 1 | CH₂-c-Pr | Me | Cl |
| 1-525 | Cl | Me | 2 | CH₂-c-Pr | Me | Cl |
| 1-526 | Cl | Cl | 0 | CH₂-c-Pr | Me | Cl |
| 1-527 | Cl | Cl | 1 | CH₂-c-Pr | Me | Cl |
| 1-528 | Cl | Cl | 2 | CH₂-c-Pr | Me | Cl |
| 1-529 | Cl | Me | 0 | CH₂-c-Pr | Me | CF₃ |
| 1-530 | Cl | Me | 1 | CH₂-c-Pr | Me | CF₃ |
| 1-531 | Cl | Me | 2 | CH₂-c-Pr | Me | CF₃ |
| 1-532 | Cl | Cl | 0 | CH₂-c-Pr | Me | CF₃ |
| 1-533 | Cl | Cl | 1 | CH₂-c-Pr | Me | CF₃ |
| 1-534 | Cl | Cl | 2 | CH₂-c-Pr | Me | CF₃ |
| 1-535 | Cl | Me | 0 | CH₂-c-Pr | Me | SO₂Me |
| 1-536 | Cl | Me | 1 | CH₂-c-Pr | Me | SO₂Me |
| 1-537 | Cl | Me | 2 | CH₂-c-Pr | Me | SO₂Me |
| 1-538 | Cl | Cl | 0 | CH₂-c-Pr | Me | SO₂Me |
| 1-539 | Cl | Cl | 1 | CH₂-c-Pr | Me | SO₂Me |
| 1-540 | Cl | Cl | 2 | CH₂-c-Pr | Me | SO₂Me |
| 1-541 | SO₂Me | Me | 0 | CH₂-c-Pr | H | H |
| 1-542 | SO₂Me | Me | 1 | CH₂-c-Pr | H | H |
| 1-543 | SO₂Me | Me | 2 | CH₂-c-Pr | H | H |
| 1-544 | SO₂Me | Cl | 0 | CH₂-c-Pr | H | H |
| 1-545 | SO₂Me | Cl | 1 | CH₂-c-Pr | H | H |
| 1-546 | SO₂Me | Cl | 2 | CH₂-c-Pr | H | H |
| 1-547 | SO₂Me | Me | 0 | CH₂-c-Pr | H | Me |
| 1-548 | SO₂Me | Me | 1 | CH₂-c-Pr | H | Me |
| 1-549 | SO₂Me | Me | 2 | CH₂-c-Pr | H | Me |
| 1-550 | SO₂Me | Cl | 0 | CH₂-c-Pr | H | Me |
| 1-551 | SO₂Me | Cl | 1 | CH₂-c-Pr | H | Me |
| 1-552 | SO₂Me | Cl | 2 | CH₂-c-Pr | H | Me |
| 1-553 | SO₂Me | Me | 0 | CH₂-c-Pr | H | Cl |
| 1-554 | SO₂Me | Me | 1 | CH₂-c-Pr | H | Cl |
| 1-555 | SO₂Me | Me | 2 | CH₂-c-Pr | H | Cl |
| 1-556 | SO₂Me | Cl | 0 | CH₂-c-Pr | H | Cl |
| 1-557 | SO₂Me | Cl | 1 | CH₂-c-Pr | H | Cl |
| 1-558 | SO₂Me | Cl | 2 | CH₂-c-Pr | H | Cl |
| 1-559 | SO₂Me | Me | 0 | CH₂-c-Pr | H | CF₃ |
| 1-560 | SO₂Me | Me | 1 | CH₂-c-Pr | H | CF₃ |
| 1-561 | SO₂Me | Me | 2 | CH₂-c-Pr | H | CF₃ |
| 1-562 | SO₂Me | Cl | 0 | CH₂-c-Pr | H | CF₃ |
| 1-563 | SO₂Me | Cl | 1 | CH₂-c-Pr | H | CF₃ |
| 1-564 | SO₂Me | Cl | 2 | CH₂-c-Pr | H | CF₃ |
| 1-565 | SO₂Me | Me | 0 | CH₂-c-Pr | H | SO₂Me |
| 1-566 | SO₂Me | Me | 1 | CH₂-c-Pr | H | SO₂Me |
| 1-567 | SO₂Me | Me | 2 | CH₂-c-Pr | H | SO₂Me |
| 1-568 | SO₂Me | Cl | 0 | CH₂-c-Pr | H | SO₂Me |
| 1-569 | SO₂Me | Cl | 1 | CH₂-c-Pr | H | SO₂Me |
| 1-570 | SO₂Me | Cl | 2 | CH₂-c-Pr | H | SO₂Me |
| 1-571 | SO₂Me | Me | 0 | CH₂-c-Pr | Me | H |
| 1-572 | SO₂Me | Me | 1 | CH₂-c-Pr | Me | H |
| 1-573 | SO₂Me | Me | 2 | CH₂-c-Pr | Me | H |
| 1-574 | SO₂Me | Cl | 0 | CH₂-c-Pr | Me | H |
| 1-575 | SO₂Me | Cl | 1 | CH₂-c-Pr | Me | H |
| 1-576 | SO₂Me | Cl | 2 | CH₂-c-Pr | Me | H |
| 1-577 | SO₂Me | Me | 0 | CH₂-c-Pr | Me | Me |
| 1-578 | SO₂Me | Me | 1 | CH₂-c-Pr | Me | Me |
| 1-579 | SO₂Me | Me | 2 | CH₂-c-Pr | Me | Me |
| 1-580 | SO₂Me | Cl | 0 | CH₂-c-Pr | Me | Me |
| 1-581 | SO₂Me | Cl | 1 | CH₂-c-Pr | Me | Me |
| 1-582 | SO₂Me | Cl | 2 | CH₂-c-Pr | Me | Me |
| 1-583 | SO₂Me | Me | 0 | CH₂-c-Pr | Me | Cl |
| 1-584 | SO₂Me | Me | 1 | CH₂-c-Pr | Me | Cl |
| 1-585 | SO₂Me | Me | 2 | CH₂-c-Pr | Me | Cl |
| 1-586 | SO₂Me | Cl | 0 | CH₂-c-Pr | Me | Cl |
| 1-587 | SO₂Me | Cl | 1 | CH₂-c-Pr | Me | Cl |
| 1-588 | SO₂Me | Cl | 2 | CH₂-c-Pr | Me | Cl |
| 1-589 | SO₂Me | Me | 0 | CH₂-c-Pr | Me | CF₃ |
| 1-590 | SO₂Me | Me | 1 | CH₂-c-Pr | Me | CF₃ |
| 1-591 | SO₂Me | Me | 2 | CH₂-c-Pr | Me | CF₃ |
| 1-592 | SO₂Me | Cl | 0 | CH₂-c-Pr | Me | CF₃ |
| 1-593 | SO₂Me | Cl | 1 | CH₂-c-Pr | Me | CF₃ |
| 1-594 | SO₂Me | Cl | 2 | CH₂-c-Pr | Me | CF₃ |
| 1-595 | SO₂Me | Me | 0 | CH₂-c-Pr | Me | SO₂Me |
| 1-596 | SO₂Me | Me | 1 | CH₂-c-Pr | Me | SO₂Me |
| 1-597 | SO₂Me | Me | 2 | CH₂-c-Pr | Me | SO₂Me |
| 1-598 | SO₂Me | Cl | 0 | CH₂-c-Pr | Me | SO₂Me |
| 1-599 | SO₂Me | Cl | 1 | CH₂-c-Pr | Me | SO₂Me |
| 1-600 | SO₂Me | Cl | 2 | CH₂-c-Pr | Me | SO₂Me |
| 1-601 | Me | Me | 0 | CH₂CH₂OMe | H | H |
| 1-602 | Me | Me | 1 | CH₂CH₂OMe | H | H |
| 1-603 | Me | Me | 2 | CH₂CH₂OMe | H | H |
| 1-604 | Me | Cl | 0 | CH₂CH₂OMe | H | H |
| 1-605 | Me | Cl | 1 | CH₂CH₂OMe | H | H |
| 1-606 | Me | Cl | 2 | CH₂CH₂OMe | H | H |
| 1-607 | Me | Me | 0 | CH₂CH₂OMe | H | Me |
| 1-608 | Me | Me | 1 | CH₂CH₂OMe | H | Me |
| 1-609 | Me | Me | 2 | CH₂CH₂OMe | H | Me |
| 1-610 | Me | Cl | 0 | CH₂CH₂OMe | H | Me |
| 1-611 | Me | Cl | 1 | CH₂CH₂OMe | H | Me |
| 1-612 | Me | Cl | 2 | CH₂CH₂OMe | H | Me |
| 1-613 | Me | Me | 0 | CH₂CH₂OMe | H | Cl |
| 1-614 | Me | Me | 1 | CH₂CH₂OMe | H | Cl |
| 1-615 | Me | Me | 2 | CH₂CH₂OMe | H | Cl |
| 1-616 | Me | Cl | 0 | CH₂CH₂OMe | H | Cl |
| 1-617 | Me | Cl | 1 | CH₂CH₂OMe | H | Cl |
| 1-618 | Me | Cl | 2 | CH₂CH₂OMe | H | Cl |
| 1-619 | Me | Me | 0 | CH₂CH₂OMe | H | CF₃ |
| 1-620 | Me | Me | 1 | CH₂CH₂OMe | H | CF₃ |
| 1-621 | Me | Me | 2 | CH₂CH₂OMe | H | CF₃ |
| 1-622 | Me | Cl | 0 | CH₂CH₂OMe | H | CF₃ |
| 1-623 | Me | Cl | 1 | CH₂CH₂OMe | H | CF₃ |
| 1-624 | Me | Cl | 2 | CH₂CH₂OMe | H | CF₃ |
| 1-625 | Me | Me | 0 | CH₂CH₂OMe | H | SO₂Me |
| 1-626 | Me | Me | 1 | CH₂CH₂OMe | H | SO₂Me |
| 1-627 | Me | Me | 2 | CH₂CH₂OMe | H | SO₂Me |
| 1-628 | Me | Cl | 0 | CH₂CH₂OMe | H | SO₂Me |
| 1-629 | Me | Cl | 1 | CH₂CH₂OMe | H | SO₂Me |
| 1-630 | Me | Cl | 2 | CH₂CH₂OMe | H | SO₂Me |
| 1-631 | Me | Me | 0 | CH₂CH₂OMe | Me | H |
| 1-632 | Me | Me | 1 | CH₂CH₂OMe | Me | H |
| 1-633 | Me | Me | 2 | CH₂CH₂OMe | Me | H |
| 1-634 | Me | Cl | 0 | CH₂CH₂OMe | Me | H |
| 1-635 | Me | Cl | 1 | CH₂CH₂OMe | Me | H |
| 1-636 | Me | Cl | 2 | CH₂CH₂OMe | Me | H |
| 1-637 | Me | Me | 0 | CH₂CH₂OMe | Me | Me |
| 1-638 | Me | Me | 1 | CH₂CH₂OMe | Me | Me |
| 1-639 | Me | Me | 2 | CH₂CH₂OMe | Me | Me |
| 1-640 | Me | Cl | 0 | CH₂CH₂OMe | Me | Me |
| 1-641 | Me | Cl | 1 | CH₂CH₂OMe | Me | Me |
| 1-642 | Me | Cl | 2 | CH₂CH₂OMe | Me | Me |
| 1-643 | Me | Me | 0 | CH₂CH₂OMe | Me | Cl |
| 1-644 | Me | Me | 1 | CH₂CH₂OMe | Me | Cl |
| 1-645 | Me | Me | 2 | CH₂CH₂OMe | Me | Cl |
| 1-646 | Me | Cl | 0 | CH₂CH₂OMe | Me | Cl |
| 1-647 | Me | Cl | 1 | CH₂CH₂OMe | Me | Cl |
| 1-648 | Me | Cl | 2 | CH₂CH₂OMe | Me | Cl |
| 1-649 | Me | Me | 0 | CH₂CH₂OMe | Me | CF₃ |
| 1-650 | Me | Me | 1 | CH₂CH₂OMe | Me | CF₃ |
| 1-651 | Me | Me | 2 | CH₂CH₂OMe | Me | CF₃ |
| 1-652 | Me | Cl | 0 | CH₂CH₂OMe | Me | CF₃ |
| 1-653 | Me | Cl | 1 | CH₂CH₂OMe | Me | CF₃ |
| 1-654 | Me | Cl | 2 | CH₂CH₂OMe | Me | CF₃ |
| 1-655 | Me | Me | 0 | CH₂CH₂OMe | Me | SO₂Me |
| 1-656 | Me | Me | 1 | CH₂CH₂OMe | Me | SO₂Me |
| 1-657 | Me | Me | 2 | CH₂CH₂OMe | Me | SO₂Me |
| 1-658 | Me | Cl | 0 | CH₂CH₂OMe | Me | SO₂Me |
| 1-659 | Me | Cl | 1 | CH₂CH₂OMe | Me | SO₂Me |
| 1-660 | Me | Cl | 2 | CH₂CH₂OMe | Me | SO₂Me |
| 1-661 | c-Pr | Me | 0 | CH₂CH₂OMe | H | H |
| 1-662 | c-Pr | Me | 1 | CH₂CH₂OMe | H | H |
| 1-663 | c-Pr | Me | 2 | CH₂CH₂OMe | H | H |
| 1-664 | c-Pr | Cl | 0 | CH₂CH₂OMe | H | H |
| 1-665 | c-Pr | Cl | 1 | CH₂CH₂OMe | H | H |
| 1-666 | c-Pr | Cl | 2 | CH₂CH₂OMe | H | H |
| 1-667 | c-Pr | Me | 0 | CH₂CH₂OMe | H | Me |
| 1-668 | c-Pr | Me | 1 | CH₂CH₂OMe | H | Me |
| 1-669 | c-Pr | Me | 2 | CH₂CH₂OMe | H | Me |
| 1-670 | c-Pr | Cl | 0 | CH₂CH₂OMe | H | Me |
| 1-671 | c-Pr | Cl | 1 | CH₂CH₂OMe | H | Me |
| 1-672 | c-Pr | Cl | 2 | CH₂CH₂OMe | H | Me |
| 1-673 | c-Pr | Me | 0 | CH₂CH₂OMe | H | Cl |
| 1-674 | c-Pr | Me | 1 | CH₂CH₂OMe | H | Cl |
| 1-675 | c-Pr | Me | 2 | CH₂CH₂OMe | H | Cl |
| 1-676 | c-Pr | Cl | 0 | CH₂CH₂OMe | H | Cl |
| 1-677 | c-Pr | Cl | 1 | CH₂CH₂OMe | H | Cl |
| 1-678 | c-Pr | Cl | 2 | CH₂CH₂OMe | H | Cl |
| 1-679 | c-Pr | Me | 0 | CH₂CH₂OMe | H | CF₃ |
| 1-680 | c-Pr | Me | 1 | CH₂CH₂OMe | H | CF₃ |
| 1-681 | c-Pr | Me | 2 | CH₂CH₂OMe | H | CF₃ |
| 1-682 | c-Pr | Cl | 0 | CH₂CH₂OMe | H | CF₃ |
| 1-683 | c-Pr | Cl | 1 | CH₂CH₂OMe | H | CF₃ |
| 1-684 | c-Pr | Cl | 2 | CH₂CH₂OMe | H | CF₃ |
| 1-685 | c-Pr | Me | 0 | CH₂CH₂OMe | H | SO₂Me |
| 1-686 | c-Pr | Me | 1 | CH₂CH₂OMe | H | SO₂Me |
| 1-687 | c-Pr | Me | 2 | CH₂CH₂OMe | H | SO₂Me |
| 1-688 | c-Pr | Cl | 0 | CH₂CH₂OMe | H | SO₂Me |
| 1-689 | c-Pr | Cl | 1 | CH₂CH₂OMe | H | SO₂Me |
| 1-690 | c-Pr | Cl | 2 | CH₂CH₂OMe | H | SO₂Me |
| 1-691 | c-Pr | Me | 0 | CH₂CH₂OMe | Me | H |
| 1-692 | c-Pr | Me | 1 | CH₂CH₂OMe | Me | H |
| 1-693 | c-Pr | Me | 2 | CH₂CH₂OMe | Me | H |
| 1-694 | c-Pr | Cl | 0 | CH₂CH₂OMe | Me | H |
| 1-695 | c-Pr | Cl | 1 | CH₂CH₂OMe | Me | H |
| 1-696 | c-Pr | Cl | 2 | CH₂CH₂OMe | Me | H |
| 1-697 | c-Pr | Me | 0 | CH₂CH₂OMe | Me | Me |
| 1-698 | c-Pr | Me | 1 | CH₂CH₂OMe | Me | Me |
| 1-699 | c-Pr | Me | 2 | CH₂CH₂OMe | Me | Me |
| 1-700 | c-Pr | Cl | 0 | CH₂CH₂OMe | Me | Me |
| 1-701 | c-Pr | Cl | 1 | CH₂CH₂OMe | Me | Me |
| 1-702 | c-Pr | Cl | 2 | CH₂CH₂OMe | Me | Me |
| 1-703 | c-Pr | Me | 0 | CH₂CH₂OMe | Me | Cl |
| 1-704 | c-Pr | Me | 1 | CH₂CH₂OMe | Me | Cl |
| 1-705 | c-Pr | Me | 2 | CH₂CH₂OMe | Me | Cl |
| 1-706 | c-Pr | Cl | 0 | CH₂CH₂OMe | Me | Cl |
| 1-707 | c-Pr | Cl | 1 | CH₂CH₂OMe | Me | Cl |
| 1-708 | c-Pr | Cl | 2 | CH₂CH₂OMe | Me | Cl |
| 1-709 | c-Pr | Me | 0 | CH₂CH₂OMe | Me | CF₃ |
| 1-710 | c-Pr | Me | 1 | CH₂CH₂OMe | Me | CF₃ |
| 1-711 | c-Pr | Me | 2 | CH₂CH₂OMe | Me | CF₃ |
| 1-712 | c-Pr | Cl | 0 | CH₂CH₂OMe | Me | CF₃ |
| 1-713 | c-Pr | Cl | 1 | CH₂CH₂OMe | Me | CF₃ |
| 1-714 | c-Pr | Cl | 2 | CH₂CH₂OMe | Me | CF₃ |
| 1-715 | c-Pr | Me | 0 | CH₂CH₂OMe | Me | SO₂Me |
| 1-716 | c-Pr | Me | 1 | CH₂CH₂OMe | Me | SO₂Me |
| 1-717 | c-Pr | Me | 2 | CH₂CH₂OMe | Me | SO₂Me |
| 1-718 | c-Pr | Cl | 0 | CH₂CH₂OMe | Me | SO₂Me |
| 1-719 | c-Pr | Cl | 1 | CH₂CH₂OMe | Me | SO₂Me |
| 1-720 | c-Pr | Cl | 2 | CH₂CH₂OMe | Me | SO₂Me |
| 1-721 | Propen-2-yl | Me | 0 | CH₂CH₂OMe | H | H |
| 1-722 | Propen-2-yl | Me | 1 | CH₂CH₂OMe | H | H |
| 1-723 | Propen-2-yl | Me | 2 | CH₂CH₂OMe | H | H |
| 1-724 | Propen-2-yl | Cl | 0 | CH₂CH₂OMe | H | H |
| 1-725 | Propen-2-yl | Cl | 1 | CH₂CH₂OMe | H | H |
| 1-726 | Propen-2-yl | Cl | 2 | CH₂CH₂OMe | H | H |
| 1-727 | Propen-2-yl | Me | 0 | CH₂CH₂OMe | H | Me |
| 1-728 | Propen-2-yl | Me | 1 | CH₂CH₂OMe | H | Me |
| 1-729 | Propen-2-yl | Me | 2 | CH₂CH₂OMe | H | Me |
| 1-730 | Propen-2-yl | Cl | 0 | CH₂CH₂OMe | H | Me |
| 1-731 | Propen-2-yl | Cl | 1 | CH₂CH₂OMe | H | Me |
| 1-732 | Propen-2-yl | Cl | 2 | CH₂CH₂OMe | H | Me |
| 1-733 | Propen-2-yl | Me | 0 | CH₂CH₂OMe | H | Cl |
| 1-734 | Propen-2-yl | Me | 1 | CH₂CH₂OMe | H | Cl |
| 1-735 | Propen-2-yl | Me | 2 | CH₂CH₂OMe | H | Cl |
| 1-736 | Propen-2-yl | Cl | 0 | CH₂CH₂OMe | H | Cl |
| 1-737 | Propen-2-yl | Cl | 1 | CH₂CH₂OMe | H | Cl |
| 1-738 | Propen-2-yl | Cl | 2 | CH₂CH₂OMe | H | Cl |
| 1-739 | Propen-2-yl | Me | 0 | CH₂CH₂OMe | H | CF₃ |
| 1-740 | Propen-2-yl | Me | 1 | CH₂CH₂OMe | H | CF₃ |
| 1-741 | Propen-2-yl | Me | 2 | CH₂CH₂OMe | H | CF₃ |
| 1-742 | Propen-2-yl | Cl | 0 | CH₂CH₂OMe | H | CF₃ |
| 1-743 | Propen-2-yl | Cl | 1 | CH₂CH₂OMe | H | CF₃ |
| 1-744 | Propen-2-yl | Cl | 2 | CH₂CH₂OMe | H | CF₃ |
| 1-745 | Propen-2-yl | Me | 0 | CH₂CH₂OMe | H | SO₂Me |
| 1-746 | Propen-2-yl | Me | 1 | CH₂CH₂OMe | H | SO₂Me |
| 1-747 | Propen-2-yl | Me | 2 | CH₂CH₂OMe | H | SO₂Me |
| 1-748 | Propen-2-yl | Cl | 0 | CH₂CH₂OMe | H | SO₂Me |
| 1-749 | Propen-2-yl | Cl | 1 | CH₂CH₂OMe | H | SO₂Me |
| 1-750 | Propen-2-yl | Cl | 2 | CH₂CH₂OMe | H | SO₂Me |
| 1-751 | Propen-2-yl | Me | 0 | CH₂CH₂OMe | Me | H |
| 1-752 | Propen-2-yl | Me | 1 | CH₂CH₂OMe | Me | H |
| 1-753 | Propen-2-yl | Me | 2 | CH₂CH₂OMe | Me | H |
| 1-754 | Propen-2-yl | Cl | 0 | CH₂CH₂OMe | Me | H |
| 1-755 | Propen-2-yl | Cl | 1 | CH₂CH₂OMe | Me | H |
| 1-756 | Propen-2-yl | Cl | 2 | CH₂CH₂OMe | Me | H |
| 1-757 | Propen-2-yl | Me | 0 | CH₂CH₂OMe | Me | Me |
| 1-758 | Propen-2-yl | Me | 1 | CH₂CH₂OMe | Me | Me |
| 1-759 | Propen-2-yl | Me | 2 | CH₂CH₂OMe | Me | Me |
| 1-760 | Propen-2-yl | Cl | 0 | CH₂CH₂OMe | Me | Me |
| 1-761 | Propen-2-yl | Cl | 1 | CH₂CH₂OMe | Me | Me |
| 1-762 | Propen-2-yl | Cl | 2 | CH₂CH₂OMe | Me | Me |
| 1-763 | Propen-2-yl | Me | 0 | CH₂CH₂OMe | Me | Cl |
| 1-764 | Propen-2-yl | Me | 1 | CH₂CH₂OMe | Me | Cl |
| 1-765 | Propen-2-yl | Me | 2 | CH₂CH₂OMe | Me | Cl |
| 1-766 | Propen-2-yl | Cl | 0 | CH₂CH₂OMe | Me | Cl |
| 1-767 | Propen-2-yl | Cl | 1 | CH₂CH₂OMe | Me | Cl |
| 1-768 | Propen-2-yl | Cl | 2 | CH₂CH₂OMe | Me | Cl |
| 1-769 | Propen-2-yl | Me | 0 | CH₂CH₂OMe | Me | CF₃ |
| 1-770 | Propen-2-yl | Me | 1 | CH₂CH₂OMe | Me | CF₃ |
| 1-771 | Propen-2-yl | Me | 2 | CH₂CH₂OMe | Me | CF₃ |
| 1-772 | Propen-2-yl | Cl | 0 | CH₂CH₂OMe | Me | CF₃ |
| 1-773 | Propen-2-yl | Cl | 1 | CH₂CH₂OMe | Me | CF₃ |
| 1-774 | Propen-2-yl | Cl | 2 | CH₂CH₂OMe | Me | CF₃ |
| 1-775 | Propen-2-yl | Me | 0 | CH₂CH₂OMe | Me | SO₂Me |
| 1-776 | Propen-2-yl | Me | 1 | CH₂CH₂OMe | Me | SO₂Me |
| 1-777 | Propen-2-yl | Me | 2 | CH₂CH₂OMe | Me | SO₂Me |
| 1-778 | Propen-2-yl | Cl | 0 | CH₂CH₂OMe | Me | SO₂Me |
| 1-779 | Propen-2-yl | Cl | 1 | CH₂CH₂OMe | Me | SO₂Me |
| 1-780 | Propen-2-yl | Cl | 2 | CH₂CH₂OMe | Me | SO₂Me |
| 1-781 | Cl | Me | 0 | CH₂CH₂OMe | H | H |
| 1-782 | Cl | Me | 1 | CH₂CH₂OMe | H | H |
| 1-783 | Cl | Me | 2 | CH₂CH₂OMe | H | H |
| 1-784 | Cl | Cl | 0 | CH₂CH₂OMe | H | H |
| 1-785 | Cl | Cl | 1 | CH₂CH₂OMe | H | H |
| 1-786 | Cl | Cl | 2 | CH₂CH₂OMe | H | H |
| 1-787 | Cl | Me | 0 | CH₂CH₂OMe | H | Me |
| 1-788 | Cl | Me | 1 | CH₂CH₂OMe | H | Me |
| 1-789 | Cl | Me | 2 | CH₂CH₂OMe | H | Me |
| 1-790 | Cl | Cl | 0 | CH₂CH₂OMe | H | Me |
| 1-791 | Cl | Cl | 1 | CH₂CH₂OMe | H | Me |
| 1-792 | Cl | Cl | 2 | CH₂CH₂OMe | H | Me |
| 1-793 | Cl | Me | 0 | CH₂CH₂OMe | H | Cl |
| 1-794 | Cl | Me | 1 | CH₂CH₂OMe | H | Cl |
| 1-795 | Cl | Me | 2 | CH₂CH₂OMe | H | Cl |
| 1-796 | Cl | Cl | 0 | CH₂CH₂OMe | H | Cl |
| 1-797 | Cl | Cl | 1 | CH₂CH₂OMe | H | Cl |
| 1-798 | Cl | Cl | 2 | CH₂CH₂OMe | H | Cl |
| 1-799 | Cl | Me | 0 | CH₂CH₂OMe | H | CF₃ |
| 1-800 | Cl | Me | 1 | CH₂CH₂OMe | H | CF₃ |
| 1-801 | Cl | Me | 2 | CH₂CH₂OMe | H | CF₃ |
| 1-802 | Cl | Cl | 0 | CH₂CH₂OMe | H | CF₃ |
| 1-803 | Cl | Cl | 1 | CH₂CH₂OMe | H | CF₃ |
| 1-804 | Cl | Cl | 2 | CH₂CH₂OMe | H | CF₃ |
| 1-805 | Cl | Me | 0 | CH₂CH₂OMe | H | SO₂Me |
| 1-806 | Cl | Me | 1 | CH₂CH₂OMe | H | SO₂Me |
| 1-807 | Cl | Me | 2 | CH₂CH₂OMe | H | SO₂Me |
| 1-808 | Cl | Cl | 0 | CH₂CH₂OMe | H | SO₂Me |
| 1-809 | Cl | Cl | 1 | CH₂CH₂OMe | H | SO₂Me |
| 1-810 | Cl | Cl | 2 | CH₂CH₂OMe | H | SO₂Me |
| 1-811 | Cl | Me | 0 | CH₂CH₂OMe | Me | H |
| 1-812 | Cl | Me | 1 | CH₂CH₂OMe | Me | H |
| 1-813 | Cl | Me | 2 | CH₂CH₂OMe | Me | H |
| 1-814 | Cl | Cl | 0 | CH₂CH₂OMe | Me | H |
| 1-815 | Cl | Cl | 1 | CH₂CH₂OMe | Me | H |
| 1-816 | Cl | Cl | 2 | CH₂CH₂OMe | Me | H |
| 1-817 | Cl | Me | 0 | CH₂CH₂OMe | Me | Me |
| 1-818 | Cl | Me | 1 | CH₂CH₂OMe | Me | Me |
| 1-819 | Cl | Me | 2 | CH₂CH₂OMe | Me | Me |
| 1-820 | Cl | Cl | 0 | CH₂CH₂OMe | Me | Me |
| 1-821 | Cl | Cl | 1 | CH₂CH₂OMe | Me | Me |
| 1-822 | Cl | Cl | 2 | CH₂CH₂OMe | Me | Me |
| 1-823 | Cl | Me | 0 | CH₂CH₂OMe | Me | Cl |
| 1-824 | Cl | Me | 1 | CH₂CH₂OMe | Me | Cl |
| 1-825 | Cl | Me | 2 | CH₂CH₂OMe | Me | Cl |
| 1-826 | Cl | Cl | 0 | CH₂CH₂OMe | Me | Cl |
| 1-827 | Cl | Cl | 1 | CH₂CH₂OMe | Me | Cl |
| 1-828 | Cl | Cl | 2 | CH₂CH₂OMe | Me | Cl |
| 1-829 | Cl | Me | 0 | CH₂CH₂OMe | Me | CF₃ |
| 1-830 | Cl | Me | 1 | CH₂CH₂OMe | Me | CF₃ |
| 1-831 | Cl | Me | 2 | CH₂CH₂OMe | Me | CF₃ |
| 1-832 | Cl | Cl | 0 | CH₂CH₂OMe | Me | CF₃ |
| 1-833 | Cl | Cl | 1 | CH₂CH₂OMe | Me | CF₃ |
| 1-834 | Cl | Cl | 2 | CH₂CH₂OMe | Me | CF₃ |
| 1-835 | Cl | Me | 0 | CH₂CH₂OMe | Me | SO₂Me |
| 1-836 | Cl | Me | 1 | CH₂CH₂OMe | Me | SO₂Me |
| 1-837 | Cl | Me | 2 | CH₂CH₂OMe | Me | SO₂Me |
| 1-838 | Cl | Cl | 0 | CH₂CH₂OMe | Me | SO₂Me |
| 1-839 | Cl | Cl | 1 | CH₂CH₂OMe | Me | SO₂Me |
| 1-840 | Cl | Cl | 2 | CH₂CH₂OMe | Me | SO₂Me |
| 1-841 | SO₂Me | Me | 0 | CH₂CH₂OMe | H | H |
| 1-842 | SO₂Me | Me | 1 | CH₂CH₂OMe | H | H |
| 1-843 | SO₂Me | Me | 2 | CH₂CH₂OMe | H | H |
| 1-844 | SO₂Me | Cl | 0 | CH₂CH₂OMe | H | H |
| 1-845 | SO₂Me | Cl | 1 | CH₂CH₂OMe | H | H |
| 1-846 | SO₂Me | Cl | 2 | CH₂CH₂OMe | H | H |
| 1-847 | SO₂Me | Me | 0 | CH₂CH₂OMe | H | Me |
| 1-848 | SO₂Me | Me | 1 | CH₂CH₂OMe | H | Me |
| 1-849 | SO₂Me | Me | 2 | CH₂CH₂OMe | H | Me |
| 1-850 | SO₂Me | Cl | 0 | CH₂CH₂OMe | H | Me |
| 1-851 | SO₂Me | Cl | 1 | CH₂CH₂OMe | H | Me |
| 1-852 | SO₂Me | Cl | 2 | CH₂CH₂OMe | H | Me |
| 1-853 | SO₂Me | Me | 0 | CH₂CH₂OMe | H | Cl |
| 1-854 | SO₂Me | Me | 1 | CH₂CH₂OMe | H | Cl |
| 1-855 | SO₂Me | Me | 2 | CH₂CH₂OMe | H | Cl |
| 1-856 | SO₂Me | Cl | 0 | CH₂CH₂OMe | H | Cl |
| 1-857 | SO₂Me | Cl | 1 | CH₂CH₂OMe | H | Cl |
| 1-858 | SO₂Me | Cl | 2 | CH₂CH₂OMe | H | Cl |
| 1-859 | SO₂Me | Me | 0 | CH₂CH₂OMe | H | CF₃ |
| 1-860 | SO₂Me | Me | 1 | CH₂CH₂OMe | H | CF₃ |
| 1-861 | SO₂Me | Me | 2 | CH₂CH₂OMe | H | CF₃ |
| 1-862 | SO₂Me | Cl | 0 | CH₂CH₂OMe | H | CF₃ |
| 1-863 | SO₂Me | Cl | 1 | CH₂CH₂OMe | H | CF₃ |
| 1-864 | SO₂Me | Cl | 2 | CH₂CH₂OMe | H | CF₃ |
| 1-865 | SO₂Me | Me | 0 | CH₂CH₂OMe | H | SO₂Me |
| 1-866 | SO₂Me | Me | 1 | CH₂CH₂OMe | H | SO₂Me |
| 1-867 | SO₂Me | Me | 2 | CH₂CH₂OMe | H | SO₂Me |
| 1-868 | SO₂Me | Cl | 0 | CH₂CH₂OMe | H | SO₂Me |
| 1-869 | SO₂Me | Cl | 1 | CH₂CH₂OMe | H | SO₂Me |
| 1-870 | SO₂Me | Cl | 2 | CH₂CH₂OMe | H | SO₂Me |
| 1-871 | SO₂Me | Me | 0 | CH₂CH₂OMe | Me | H |
| 1-872 | SO₂Me | Me | 1 | CH₂CH₂OMe | Me | H |
| 1-873 | SO₂Me | Me | 2 | CH₂CH₂OMe | Me | H |
| 1-874 | SO₂Me | Cl | 0 | CH₂CH₂OMe | Me | H |
| 1-875 | SO₂Me | Cl | 1 | CH₂CH₂OMe | Me | H |
| 1-876 | SO₂Me | Cl | 2 | CH₂CH₂OMe | Me | H |
| 1-877 | SO₂Me | Me | 0 | CH₂CH₂OMe | Me | Me |
| 1-878 | SO₂Me | Me | 1 | CH₂CH₂OMe | Me | Me |
| 1-879 | SO₂Me | Me | 2 | CH₂CH₂OMe | Me | Me |
| 1-880 | SO₂Me | Cl | 0 | CH₂CH₂OMe | Me | Me |
| 1-881 | SO₂Me | Cl | 1 | CH₂CH₂OMe | Me | Me |
| 1-882 | SO₂Me | Cl | 2 | CH₂CH₂OMe | Me | Me |
| 1-883 | SO₂Me | Me | 0 | CH₂CH₂OMe | Me | Cl |
| 1-884 | SO₂Me | Me | 1 | CH₂CH₂OMe | Me | Cl |
| 1-885 | SO₂Me | Me | 2 | CH₂CH₂OMe | Me | Cl |
| 1-886 | SO₂Me | Cl | 0 | CH₂CH₂OMe | Me | Cl |
| 1-887 | SO₂Me | Cl | 1 | CH₂CH₂OMe | Me | Cl |
| 1-888 | SO₂Me | Cl | 2 | CH₂CH₂OMe | Me | Cl |
| 1-889 | SO₂Me | Me | 0 | CH₂CH₂OMe | Me | CF₃ |
| 1-890 | SO₂Me | Me | 1 | CH₂CH₂OMe | Me | CF₃ |
| 1-891 | SO₂Me | Me | 2 | CH₂CH₂OMe | Me | CF₃ |
| 1-892 | SO₂Me | Cl | 0 | CH₂CH₂OMe | Me | CF₃ |
| 1-893 | SO₂Me | Cl | 1 | CH₂CH₂OMe | Me | CF₃ |
| 1-894 | SO₂Me | Cl | 2 | CH₂CH₂OMe | Me | CF₃ |
| 1-895 | SO₂Me | Me | 0 | CH₂CH₂OMe | Me | SO₂Me |
| 1-896 | SO₂Me | Me | 1 | CH₂CH₂OMe | Me | SO₂Me |
| 1-897 | SO₂Me | Me | 2 | CH₂CH₂OMe | Me | SO₂Me |
| 1-898 | SO₂Me | Cl | 0 | CH₂CH₂OMe | Me | SO₂Me |
| 1-899 | SO₂Me | Cl | 1 | CH₂CH₂OMe | Me | SO₂Me |
| 1-900 | SO₂Me | Cl | 2 | CH₂CH₂OMe | Me | SO₂Me |

TABLE 2
Compounds according to the invention of the general formula (I) in
which R² and R³ each represent hydrogen and A represents —CH₂—,
X¹ represents CH, X² represents CR⁷ and X³ represents CR⁸

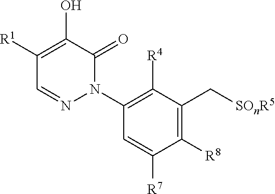

| No. | R¹ | R⁴ | n | R⁵ | R⁷ | R⁸ |
|---|---|---|---|---|---|---|
| 2-1 | Me | Me | 0 | Me | H | H |
| 2-2 | Me | Me | 1 | Me | H | H |
| 2-3 | Me | Me | 2 | Me | H | H |
| 2-4 | Me | Cl | 0 | Me | H | H |
| 2-5 | Me | Cl | 1 | Me | H | H |
| 2-6 | Me | Cl | 2 | Me | H | H |
| 2-7 | Me | Me | 0 | Me | H | Me |
| 2-8 | Me | Me | 1 | Me | H | Me |
| 2-9 | Me | Me | 2 | Me | H | Me |
| 2-10 | Me | Cl | 0 | Me | H | Me |
| 2-11 | Me | Cl | 1 | Me | H | Me |
| 2-12 | Me | Cl | 2 | Me | H | Me |
| 2-13 | Me | Me | 0 | Me | H | Cl |
| 2-14 | Me | Me | 1 | Me | H | Cl |
| 2-15 | Me | Me | 2 | Me | H | Cl |
| 2-16 | Me | Cl | 0 | Me | H | Cl |
| 2-17 | Me | Cl | 1 | Me | H | Cl |
| 2-18 | Me | Cl | 2 | Me | H | Cl |
| 2-19 | Me | Me | 0 | Me | H | CF₃ |
| 2-20 | Me | Me | 1 | Me | H | CF₃ |
| 2-21 | Me | Me | 2 | Me | H | CF₃ |
| 2-22 | Me | Cl | 0 | Me | H | CF₃ |
| 2-23 | Me | Cl | 1 | Me | H | CF₃ |
| 2-24 | Me | Cl | 2 | Me | H | CF₃ |
| 2-25 | Me | Me | 0 | Me | H | SO₂Me |
| 2-26 | Me | Me | 1 | Me | H | SO₂Me |
| 2-27 | Me | Me | 2 | Me | H | SO₂Me |
| 2-28 | Me | Cl | 0 | Me | H | SO₂Me |
| 2-29 | Me | Cl | 1 | Me | H | SO₂Me |
| 2-30 | Me | Cl | 2 | Me | H | SO₂Me |
| 2-31 | Me | Me | 0 | Me | Me | H |
| 2-32 | Me | Me | 1 | Me | Me | H |
| 2-33 | Me | Me | 2 | Me | Me | H |
| 2-34 | Me | Cl | 0 | Me | Me | H |
| 2-35 | Me | Cl | 1 | Me | Me | H |
| 2-36 | Me | Cl | 2 | Me | Me | H |
| 2-37 | Me | Me | 0 | Me | Me | Me |
| 2-38 | Me | Me | 1 | Me | Me | Me |
| 2-39 | Me | Me | 2 | Me | Me | Me |
| 2-40 | Me | Cl | 0 | Me | Me | Me |
| 2-41 | Me | Cl | 1 | Me | Me | Me |
| 2-42 | Me | Cl | 2 | Me | Me | Me |
| 2-43 | Me | Me | 0 | Me | Me | Cl |
| 2-44 | Me | Me | 1 | Me | Me | Cl |
| 2-45 | Me | Me | 2 | Me | Me | Cl |
| 2-46 | Me | Cl | 0 | Me | Me | Cl |
| 2-47 | Me | Cl | 1 | Me | Me | Cl |
| 2-48 | Me | Cl | 2 | Me | Me | Cl |
| 2-49 | Me | Me | 0 | Me | Me | CF₃ |
| 2-50 | Me | Me | 1 | Me | Me | CF₃ |
| 2-51 | Me | Me | 2 | Me | Me | CF₃ |
| 2-52 | Me | Cl | 0 | Me | Me | CF₃ |
| 2-53 | Me | Cl | 1 | Me | Me | CF₃ |
| 2-54 | Me | Cl | 2 | Me | Me | CF₃ |
| 2-55 | Me | Me | 0 | Me | Me | SO₂Me |
| 2-56 | Me | Me | 1 | Me | Me | SO₂Me |
| 2-57 | Me | Me | 2 | Me | Me | SO₂Me |
| 2-58 | Me | Cl | 0 | Me | Me | SO₂Me |
| 2-59 | Me | Cl | 1 | Me | Me | SO₂Me |
| 2-60 | Me | Cl | 2 | Me | Me | SO₂Me |
| 2-61 | c-Pr | Me | 0 | Me | H | H |
| 2-62 | c-Pr | Me | 1 | Me | H | H |
| 2-63 | c-Pr | Me | 2 | Me | H | H |
| 2-64 | c-Pr | Cl | 0 | Me | H | H |
| 2-65 | c-Pr | Cl | 1 | Me | H | H |
| 2-66 | c-Pr | Cl | 2 | Me | H | H |
| 2-67 | c-Pr | Me | 0 | Me | H | Me |
| 2-68 | c-Pr | Me | 1 | Me | H | Me |
| 2-69 | c-Pr | Me | 2 | Me | H | Me |
| 2-70 | c-Pr | Cl | 0 | Me | H | Me |
| 2-71 | c-Pr | Cl | 1 | Me | H | Me |
| 2-72 | c-Pr | Cl | 2 | Me | H | Me |
| 2-73 | c-Pr | Me | 0 | Me | H | Cl |
| 2-74 | c-Pr | Me | 1 | Me | H | Cl |
| 2-75 | c-Pr | Me | 2 | Me | H | Cl |
| 2-76 | c-Pr | Cl | 0 | Me | H | Cl |
| 2-77 | c-Pr | Cl | 1 | Me | H | Cl |
| 2-78 | c-Pr | Cl | 2 | Me | H | Cl |
| 2-79 | c-Pr | Me | 0 | Me | H | CF₃ |
| 2-80 | c-Pr | Me | 1 | Me | H | CF₃ |
| 2-81 | c-Pr | Me | 2 | Me | H | CF₃ |
| 2-82 | c-Pr | Cl | 0 | Me | H | CF₃ |
| 2-83 | c-Pr | Cl | 1 | Me | H | CF₃ |
| 2-84 | c-Pr | Cl | 2 | Me | H | CF₃ |
| 2-85 | c-Pr | Me | 0 | Me | H | SO₂Me |
| 2-86 | c-Pr | Me | 1 | Me | H | SO₂Me |
| 2-87 | c-Pr | Me | 2 | Me | H | SO₂Me |
| 2-88 | c-Pr | Cl | 0 | Me | H | SO₂Me |
| 2-89 | c-Pr | Cl | 1 | Me | H | SO₂Me |
| 2-90 | c-Pr | Cl | 2 | Me | H | SO₂Me |
| 2-91 | c-Pr | Me | 0 | Me | Me | H |
| 2-92 | c-Pr | Me | 1 | Me | Me | H |
| 2-93 | c-Pr | Me | 2 | Me | Me | H |
| 2-94 | c-Pr | Cl | 0 | Me | Me | H |
| 2-95 | c-Pr | Cl | 1 | Me | Me | H |
| 2-96 | c-Pr | Cl | 2 | Me | Me | H |
| 2-97 | c-Pr | Me | 0 | Me | Me | Me |
| 2-98 | c-Pr | Me | 1 | Me | Me | Me |
| 2-99 | c-Pr | Me | 2 | Me | Me | Me |
| 2-100 | c-Pr | Cl | 0 | Me | Me | Me |
| 2-101 | c-Pr | Cl | 1 | Me | Me | Me |
| 2-102 | c-Pr | Cl | 2 | Me | Me | Me |
| 2-103 | c-Pr | Me | 0 | Me | Me | Cl |
| 2-104 | c-Pr | Me | 1 | Me | Me | Cl |
| 2-105 | c-Pr | Me | 2 | Me | Me | Cl |
| 2-106 | c-Pr | Cl | 0 | Me | Me | Cl |
| 2-107 | c-Pr | Cl | 1 | Me | Me | Cl |
| 2-108 | c-Pr | Cl | 2 | Me | Me | Cl |
| 2-109 | c-Pr | Me | 0 | Me | Me | CF₃ |
| 2-110 | c-Pr | Me | 1 | Me | Me | CF₃ |
| 2-111 | c-Pr | Me | 2 | Me | Me | CF₃ |
| 2-112 | c-Pr | Cl | 0 | Me | Me | CF₃ |
| 2-113 | c-Pr | Cl | 1 | Me | Me | CF₃ |
| 2-114 | c-Pr | Cl | 2 | Me | Me | CF₃ |
| 2-115 | c-Pr | Me | 0 | Me | Me | SO₂Me |
| 2-116 | c-Pr | Me | 1 | Me | Me | SO₂Me |
| 2-117 | c-Pr | Me | 2 | Me | Me | SO₂Me |
| 2-118 | c-Pr | Cl | 0 | Me | Me | SO₂Me |
| 2-119 | c-Pr | Cl | 1 | Me | Me | SO₂Me |
| 2-120 | c-Pr | Cl | 2 | Me | Me | SO₂Me |
| 2-121 | Cl | Me | 0 | Me | H | H |
| 2-122 | Cl | Me | 1 | Me | H | H |
| 2-123 | Cl | Me | 2 | Me | H | H |
| 2-124 | Cl | Cl | 0 | Me | H | H |
| 2-125 | Cl | Cl | 1 | Me | H | H |
| 2-126 | Cl | Cl | 2 | Me | H | H |
| 2-127 | Cl | Me | 0 | Me | H | Me |
| 2-128 | Cl | Me | 1 | Me | H | Me |
| 2-129 | Cl | Me | 2 | Me | H | Me |
| 2-130 | Cl | Cl | 0 | Me | H | Me |
| 2-131 | Cl | Cl | 1 | Me | H | Me |
| 2-132 | Cl | Cl | 2 | Me | H | Me |
| 2-133 | Cl | Me | 0 | Me | H | Cl |
| 2-134 | Cl | Me | 1 | Me | H | Cl |
| 2-135 | Cl | Me | 2 | Me | H | Cl |
| 2-136 | Cl | Cl | 0 | Me | H | Cl |
| 2-137 | Cl | Cl | 1 | Me | H | Cl |
| 2-138 | Cl | Cl | 2 | Me | H | Cl |
| 2-139 | Cl | Me | 0 | Me | H | CF₃ |
| 2-140 | Cl | Me | 1 | Me | H | CF₃ |
| 2-141 | Cl | Me | 2 | Me | H | CF₃ |
| 2-142 | Cl | Cl | 0 | Me | H | CF₃ |
| 2-143 | Cl | Cl | 1 | Me | H | CF₃ |
| 2-144 | Cl | Cl | 2 | Me | H | CF₃ |
| 2-145 | Cl | Me | 0 | Me | H | SO₂Me |
| 2-146 | Cl | Me | 1 | Me | H | SO₂Me |
| 2-147 | Cl | Me | 2 | Me | H | SO₂Me |
| 2-148 | Cl | Cl | 0 | Me | H | SO₂Me |
| 2-149 | Cl | Cl | 1 | Me | H | SO₂Me |
| 2-150 | Cl | Cl | 2 | Me | H | SO₂Me |
| 2-151 | Cl | Me | 0 | Me | Me | H |
| 2-152 | Cl | Me | 1 | Me | Me | H |
| 2-153 | Cl | Me | 2 | Me | Me | H |
| 2-154 | Cl | Cl | 0 | Me | Me | H |
| 2-155 | Cl | Cl | 1 | Me | Me | H |
| 2-156 | Cl | Cl | 2 | Me | Me | H |
| 2-157 | Cl | Me | 0 | Me | Me | Me |
| 2-158 | Cl | Me | 1 | Me | Me | Me |
| 2-159 | Cl | Me | 2 | Me | Me | Me |
| 2-160 | Cl | Cl | 0 | Me | Me | Me |
| 2-161 | Cl | Cl | 1 | Me | Me | Me |
| 2-162 | Cl | Cl | 2 | Me | Me | Me |
| 2-163 | Cl | Me | 0 | Me | Me | Cl |
| 2-164 | Cl | Me | 1 | Me | Me | Cl |
| 2-165 | Cl | Me | 2 | Me | Me | Cl |
| 2-166 | Cl | Cl | 0 | Me | Me | Cl |
| 2-167 | Cl | Cl | 1 | Me | Me | Cl |
| 2-168 | Cl | Cl | 2 | Me | Me | Cl |
| 2-169 | Cl | Me | 0 | Me | Me | CF₃ |
| 2-170 | Cl | Me | 1 | Me | Me | CF₃ |
| 2-171 | Cl | Me | 2 | Me | Me | CF₃ |
| 2-172 | Cl | Cl | 0 | Me | Me | CF₃ |
| 2-173 | Cl | Cl | 1 | Me | Me | CF₃ |
| 2-174 | Cl | Cl | 2 | Me | Me | CF₃ |
| 2-175 | Cl | Me | 0 | Me | Me | SO₂Me |
| 2-176 | Cl | Me | 1 | Me | Me | SO₂Me |
| 2-177 | Cl | Me | 2 | Me | Me | SO₂Me |
| 2-178 | Cl | Cl | 0 | Me | Me | SO₂Me |
| 2-179 | Cl | Cl | 1 | Me | Me | SO₂Me |
| 2-180 | Cl | Cl | 2 | Me | Me | SO₂Me |
| 2-181 | SO₂Me | Me | 0 | Me | H | H |
| 2-182 | SO₂Me | Me | 1 | Me | H | H |
| 2-183 | SO₂Me | Me | 2 | Me | H | H |
| 2-184 | SO₂Me | Cl | 0 | Me | H | H |
| 2-185 | SO₂Me | Cl | 1 | Me | H | H |
| 2-186 | SO₂Me | Cl | 2 | Me | H | H |
| 2-187 | SO₂Me | Me | 0 | Me | H | Me |
| 2-188 | SO₂Me | Me | 1 | Me | H | Me |
| 2-189 | SO₂Me | Me | 2 | Me | H | Me |
| 2-190 | SO₂Me | Cl | 0 | Me | H | Me |
| 2-191 | SO₂Me | Cl | 1 | Me | H | Me |
| 2-192 | SO₂Me | Cl | 2 | Me | H | Me |
| 2-193 | SO₂Me | Me | 0 | Me | H | Cl |
| 2-194 | SO₂Me | Me | 1 | Me | H | Cl |
| 2-195 | SO₂Me | Me | 2 | Me | H | Cl |
| 2-196 | SO₂Me | Cl | 0 | Me | H | Cl |
| 2-197 | SO₂Me | Cl | 1 | Me | H | Cl |
| 2-198 | SO₂Me | Cl | 2 | Me | H | Cl |
| 2-199 | SO₂Me | Me | 0 | Me | H | CF₃ |
| 2-200 | SO₂Me | Me | 1 | Me | H | CF₃ |
| 2-201 | SO₂Me | Me | 2 | Me | H | CF₃ |
| 2-202 | SO₂Me | Cl | 0 | Me | H | CF₃ |
| 2-203 | SO₂Me | Cl | 1 | Me | H | CF₃ |
| 2-204 | SO₂Me | Cl | 2 | Me | H | CF₃ |
| 2-205 | SO₂Me | Me | 0 | Me | H | SO₂Me |
| 2-206 | SO₂Me | Me | 1 | Me | H | SO₂Me |
| 2-207 | SO₂Me | Me | 2 | Me | H | SO₂Me |
| 2-208 | SO₂Me | Cl | 0 | Me | H | SO₂Me |
| 2-209 | SO₂Me | Cl | 1 | Me | H | SO₂Me |
| 2-210 | SO₂Me | Cl | 2 | Me | H | SO₂Me |
| 2-211 | SO₂Me | Me | 0 | Me | Me | H |
| 2-212 | SO₂Me | Me | 1 | Me | Me | H |
| 2-213 | SO₂Me | Me | 2 | Me | Me | H |
| 2-214 | SO₂Me | Cl | 0 | Me | Me | H |
| 2-215 | SO₂Me | Cl | 1 | Me | Me | H |
| 2-216 | SO₂Me | Cl | 2 | Me | Me | H |
| 2-217 | SO₂Me | Me | 0 | Me | Me | Me |
| 2-218 | SO₂Me | Me | 1 | Me | Me | Me |
| 2-219 | SO₂Me | Me | 2 | Me | Me | Me |
| 2-220 | SO₂Me | Cl | 0 | Me | Me | Me |
| 2-221 | SO₂Me | Cl | 1 | Me | Me | Me |
| 2-222 | SO₂Me | Cl | 2 | Me | Me | Me |
| 2-223 | SO₂Me | Me | 0 | Me | Me | Cl |
| 2-224 | SO₂Me | Me | 1 | Me | Me | Cl |
| 2-225 | SO₂Me | Me | 2 | Me | Me | Cl |
| 2-226 | SO₂Me | Cl | 0 | Me | Me | Cl |
| 2-227 | SO₂Me | Cl | 1 | Me | Me | Cl |
| 2-228 | SO₂Me | Cl | 2 | Me | Me | Cl |
| 2-229 | SO₂Me | Me | 0 | Me | Me | CF₃ |
| 2-230 | SO₂Me | Me | 1 | Me | Me | CF₃ |
| 2-231 | SO₂Me | Me | 2 | Me | Me | CF₃ |
| 2-232 | SO₂Me | Cl | 0 | Me | Me | CF₃ |
| 2-233 | SO₂Me | Cl | 1 | Me | Me | CF₃ |
| 2-234 | SO₂Me | Cl | 2 | Me | Me | CF₃ |
| 2-235 | SO₂Me | Me | 0 | Me | Me | SO₂Me |
| 2-236 | SO₂Me | Me | 1 | Me | Me | SO₂Me |
| 2-237 | SO₂Me | Me | 2 | Me | Me | SO₂Me |
| 2-238 | SO₂Me | Cl | 0 | Me | Me | SO₂Me |
| 2-239 | SO₂Me | Cl | 1 | Me | Me | SO₂Me |
| 2-240 | SO₂Me | Cl | 2 | Me | Me | SO₂Me |
| 2-241 | Me | Me | 0 | CH₂CH₂OMe | H | H |
| 2-242 | Me | Me | 1 | CH₂CH₂OMe | H | H |
| 2-243 | Me | Me | 2 | CH₂CH₂OMe | H | H |
| 2-244 | Me | Cl | 0 | CH₂CH₂OMe | H | H |
| 2-245 | Me | Cl | 1 | CH₂CH₂OMe | H | H |
| 2-246 | Me | Cl | 2 | CH₂CH₂OMe | H | H |
| 2-247 | Me | Me | 0 | CH₂CH₂OMe | H | Me |
| 2-248 | Me | Me | 1 | CH₂CH₂OMe | H | Me |
| 2-249 | Me | Me | 2 | CH₂CH₂OMe | H | Me |
| 2-250 | Me | Cl | 0 | CH₂CH₂OMe | H | Me |
| 2-251 | Me | Cl | 1 | CH₂CH₂OMe | H | Me |
| 2-252 | Me | Cl | 2 | CH₂CH₂OMe | H | Me |
| 2-253 | Me | Me | 0 | CH₂CH₂OMe | H | Cl |
| 2-254 | Me | Me | 1 | CH₂CH₂OMe | H | Cl |
| 2-255 | Me | Me | 2 | CH₂CH₂OMe | H | Cl |
| 2-256 | Me | Cl | 0 | CH₂CH₂OMe | H | Cl |
| 2-257 | Me | Cl | 1 | CH₂CH₂OMe | H | Cl |
| 2-258 | Me | Cl | 2 | CH₂CH₂OMe | H | Cl |
| 2-259 | Me | Me | 0 | CH₂CH₂OMe | H | CF₃ |
| 2-260 | Me | Me | 1 | CH₂CH₂OMe | H | CF₃ |
| 2-261 | Me | Me | 2 | CH₂CH₂OMe | H | CF₃ |
| 2-262 | Me | Cl | 0 | CH₂CH₂OMe | H | CF₃ |
| 2-263 | Me | Cl | 1 | CH₂CH₂OMe | H | CF₃ |
| 2-264 | Me | Cl | 2 | CH₂CH₂OMe | H | CF₃ |
| 2-265 | Me | Me | 0 | CH₂CH₂OMe | H | SO₂Me |
| 2-266 | Me | Me | 1 | CH₂CH₂OMe | H | SO₂Me |
| 2-267 | Me | Me | 2 | CH₂CH₂OMe | H | SO₂Me |
| 2-268 | Me | Cl | 0 | CH₂CH₂OMe | H | SO₂Me |
| 2-269 | Me | Cl | 1 | CH₂CH₂OMe | H | SO₂Me |
| 2-270 | Me | Cl | 2 | CH₂CH₂OMe | H | SO₂Me |
| 2-271 | Me | Me | 0 | CH₂CH₂OMe | Me | H |
| 2-272 | Me | Me | 1 | CH₂CH₂OMe | Me | H |
| 2-273 | Me | Me | 2 | CH₂CH₂OMe | Me | H |
| 2-274 | Me | Cl | 0 | CH₂CH₂OMe | Me | H |
| 2-275 | Me | Cl | 1 | CH₂CH₂OMe | Me | H |
| 2-276 | Me | Cl | 2 | CH₂CH₂OMe | Me | H |
| 2-277 | Me | Me | 0 | CH₂CH₂OMe | Me | Me |
| 2-278 | Me | Me | 1 | CH₂CH₂OMe | Me | Me |
| 2-279 | Me | Me | 2 | CH₂CH₂OMe | Me | Me |
| 2-280 | Me | Cl | 0 | CH₂CH₂OMe | Me | Me |
| 2-281 | Me | Cl | 1 | CH₂CH₂OMe | Me | Me |
| 2-282 | Me | Cl | 2 | CH₂CH₂OMe | Me | Me |
| 2-283 | Me | Me | 0 | CH₂CH₂OMe | Me | Cl |
| 2-284 | Me | Me | 1 | CH₂CH₂OMe | Me | Cl |
| 2-285 | Me | Me | 2 | CH₂CH₂OMe | Me | Cl |
| 2-286 | Me | Cl | 0 | CH₂CH₂OMe | Me | Cl |
| 2-287 | Me | Cl | 1 | CH₂CH₂OMe | Me | Cl |
| 2-288 | Me | Cl | 2 | CH₂CH₂OMe | Me | Cl |
| 2-289 | Me | Me | 0 | CH₂CH₂OMe | Me | CF₃ |
| 2-290 | Me | Me | 1 | CH₂CH₂OMe | Me | CF₃ |
| 2-291 | Me | Me | 2 | CH₂CH₂OMe | Me | CF₃ |
| 2-292 | Me | Cl | 0 | CH₂CH₂OMe | Me | CF₃ |
| 2-293 | Me | Cl | 1 | CH₂CH₂OMe | Me | CF₃ |
| 2-294 | Me | Cl | 2 | CH₂CH₂OMe | Me | CF₃ |
| 2-295 | Me | Me | 0 | CH₂CH₂OMe | Me | SO₂Me |
| 2-296 | Me | Me | 1 | CH₂CH₂OMe | Me | SO₂Me |
| 2-297 | Me | Me | 2 | CH₂CH₂OMe | Me | SO₂Me |
| 2-298 | Me | Cl | 0 | CH₂CH₂OMe | Me | SO₂Me |
| 2-299 | Me | Cl | 1 | CH₂CH₂OMe | Me | SO₂Me |
| 2-300 | Me | Cl | 2 | CH₂CH₂OMe | Me | SO₂Me |
| 2-301 | c-Pr | Me | 0 | CH₂CH₂OMe | H | H |
| 2-302 | c-Pr | Me | 1 | CH₂CH₂OMe | H | H |
| 2-303 | c-Pr | Me | 2 | CH₂CH₂OMe | H | H |
| 2-304 | c-Pr | Cl | 0 | CH₂CH₂OMe | H | H |
| 2-305 | c-Pr | Cl | 1 | CH₂CH₂OMe | H | H |
| 2-306 | c-Pr | Cl | 2 | CH₂CH₂OMe | H | H |
| 2-307 | c-Pr | Me | 0 | CH₂CH₂OMe | H | Me |
| 2-308 | c-Pr | Me | 1 | CH₂CH₂OMe | H | Me |
| 2-309 | c-Pr | Me | 2 | CH₂CH₂OMe | H | Me |
| 2-310 | c-Pr | Cl | 0 | CH₂CH₂OMe | H | Me |
| 2-311 | c-Pr | Cl | 1 | CH₂CH₂OMe | H | Me |
| 2-312 | c-Pr | Cl | 2 | CH₂CH₂OMe | H | Me |
| 2-313 | c-Pr | Me | 0 | CH₂CH₂OMe | H | Cl |
| 2-314 | c-Pr | Me | 1 | CH₂CH₂OMe | H | Cl |
| 2-315 | c-Pr | Me | 2 | CH₂CH₂OMe | H | Cl |
| 2-316 | c-Pr | Cl | 0 | CH₂CH₂OMe | H | Cl |
| 2-317 | c-Pr | Cl | 1 | CH₂CH₂OMe | H | Cl |
| 2-318 | c-Pr | Cl | 2 | CH₂CH₂OMe | H | Cl |
| 2-319 | c-Pr | Me | 0 | CH₂CH₂OMe | H | CF₃ |
| 2-320 | c-Pr | Me | 1 | CH₂CH₂OMe | H | CF₃ |
| 2-321 | c-Pr | Me | 2 | CH₂CH₂OMe | H | CF₃ |
| 2-322 | c-Pr | Cl | 0 | CH₂CH₂OMe | H | CF₃ |
| 2-323 | c-Pr | Cl | 1 | CH₂CH₂OMe | H | CF₃ |
| 2-324 | c-Pr | Cl | 2 | CH₂CH₂OMe | H | CF₃ |
| 2-325 | c-Pr | Me | 0 | CH₂CH₂OMe | H | SO₂Me |
| 2-326 | c-Pr | Me | 1 | CH₂CH₂OMe | H | SO₂Me |
| 2-327 | c-Pr | Me | 2 | CH₂CH₂OMe | H | SO₂Me |
| 2-328 | c-Pr | Cl | 0 | CH₂CH₂OMe | H | SO₂Me |
| 2-329 | c-Pr | Cl | 1 | CH₂CH₂OMe | H | SO₂Me |
| 2-330 | c-Pr | Cl | 2 | CH₂CH₂OMe | H | SO₂Me |
| 2-331 | c-Pr | Me | 0 | CH₂CH₂OMe | Me | H |
| 2-332 | c-Pr | Me | 1 | CH₂CH₂OMe | Me | H |
| 2-333 | c-Pr | Me | 2 | CH₂CH₂OMe | Me | H |
| 2-334 | c-Pr | Cl | 0 | CH₂CH₂OMe | Me | H |
| 2-335 | c-Pr | Cl | 1 | CH₂CH₂OMe | Me | H |
| 2-336 | c-Pr | Cl | 2 | CH₂CH₂OMe | Me | H |
| 2-337 | c-Pr | Me | 0 | CH₂CH₂OMe | Me | Me |
| 2-338 | c-Pr | Me | 1 | CH₂CH₂OMe | Me | Me |
| 2-339 | c-Pr | Me | 2 | CH₂CH₂OMe | Me | Me |
| 2-340 | c-Pr | Cl | 0 | CH₂CH₂OMe | Me | Me |
| 2-341 | c-Pr | Cl | 1 | CH₂CH₂OMe | Me | Me |
| 2-342 | c-Pr | Cl | 2 | CH₂CH₂OMe | Me | Me |
| 2-343 | c-Pr | Me | 0 | CH₂CH₂OMe | Me | Cl |
| 2-344 | c-Pr | Me | 1 | CH₂CH₂OMe | Me | Cl |
| 2-345 | c-Pr | Me | 2 | CH₂CH₂OMe | Me | Cl |
| 2-346 | c-Pr | Cl | 0 | CH₂CH₂OMe | Me | Cl |
| 2-347 | c-Pr | Cl | 1 | CH₂CH₂OMe | Me | Cl |
| 2-348 | c-Pr | Cl | 2 | CH₂CH₂OMe | Me | Cl |
| 2-349 | c-Pr | Me | 0 | CH₂CH₂OMe | Me | CF₃ |
| 2-350 | c-Pr | Me | 1 | CH₂CH₂OMe | Me | CF₃ |
| 2-351 | c-Pr | Me | 2 | CH₂CH₂OMe | Me | CF₃ |
| 2-352 | c-Pr | Cl | 0 | CH₂CH₂OMe | Me | CF₃ |
| 2-353 | c-Pr | Cl | 1 | CH₂CH₂OMe | Me | CF₃ |
| 2-354 | c-Pr | Cl | 2 | CH₂CH₂OMe | Me | CF₃ |
| 2-355 | c-Pr | Me | 0 | CH₂CH₂OMe | Me | SO₂Me |
| 2-356 | c-Pr | Me | 1 | CH₂CH₂OMe | Me | SO₂Me |
| 2-357 | c-Pr | Me | 2 | CH₂CH₂OMe | Me | SO₂Me |
| 2-358 | c-Pr | Cl | 0 | CH₂CH₂OMe | Me | SO₂Me |
| 2-359 | c-Pr | Cl | 1 | CH₂CH₂OMe | Me | SO₂Me |
| 2-360 | c-Pr | Cl | 2 | CH₂CH₂OMe | Me | SO₂Me |
| 2-361 | Cl | Me | 0 | CH₂CH₂OMe | H | H |
| 2-362 | Cl | Me | 1 | CH₂CH₂OMe | H | H |
| 2-363 | Cl | Me | 2 | CH₂CH₂OMe | H | H |
| 2-364 | Cl | Cl | 0 | CH₂CH₂OMe | H | H |
| 2-365 | Cl | Cl | 1 | CH₂CH₂OMe | H | H |
| 2-366 | Cl | Cl | 2 | CH₂CH₂OMe | H | H |
| 2-367 | Cl | Me | 0 | CH₂CH₂OMe | H | Me |
| 2-368 | Cl | Me | 1 | CH₂CH₂OMe | H | Me |
| 2-369 | Cl | Me | 2 | CH₂CH₂OMe | H | Me |
| 2-370 | Cl | Cl | 0 | CH₂CH₂OMe | H | Me |
| 2-371 | Cl | Cl | 1 | CH₂CH₂OMe | H | Me |
| 2-372 | Cl | Cl | 2 | CH₂CH₂OMe | H | Me |
| 2-373 | Cl | Me | 0 | CH₂CH₂OMe | H | Cl |
| 2-374 | Cl | Me | 1 | CH₂CH₂OMe | H | Cl |
| 2-375 | Cl | Me | 2 | CH₂CH₂OMe | H | Cl |
| 2-376 | Cl | Cl | 0 | CH₂CH₂OMe | H | Cl |
| 2-377 | Cl | Cl | 1 | CH₂CH₂OMe | H | Cl |
| 2-378 | Cl | Cl | 2 | CH₂CH₂OMe | H | Cl |
| 2-379 | Cl | Me | 0 | CH₂CH₂OMe | H | CF₃ |
| 2-380 | Cl | Me | 1 | CH₂CH₂OMe | H | CF₃ |
| 2-381 | Cl | Me | 2 | CH₂CH₂OMe | H | CF₃ |
| 2-382 | Cl | Cl | 0 | CH₂CH₂OMe | H | CF₃ |
| 2-383 | Cl | Cl | 1 | CH₂CH₂OMe | H | CF₃ |
| 2-384 | Cl | Cl | 2 | CH₂CH₂OMe | H | CF₃ |
| 2-385 | Cl | Me | 0 | CH₂CH₂OMe | H | SO₂Me |
| 2-386 | Cl | Me | 1 | CH₂CH₂OMe | H | SO₂Me |
| 2-387 | Cl | Me | 2 | CH₂CH₂OMe | H | SO₂Me |
| 2-388 | Cl | Cl | 0 | CH₂CH₂OMe | H | SO₂Me |
| 2-389 | Cl | Cl | 1 | CH₂CH₂OMe | H | SO₂Me |
| 2-390 | Cl | Cl | 2 | CH₂CH₂OMe | H | SO₂Me |
| 2-391 | Cl | Me | 0 | CH₂CH₂OMe | Me | H |
| 2-392 | Cl | Me | 1 | CH₂CH₂OMe | Me | H |
| 2-393 | Cl | Me | 2 | CH₂CH₂OMe | Me | H |
| 2-394 | Cl | Cl | 0 | CH₂CH₂OMe | Me | H |
| 2-395 | Cl | Cl | 1 | CH₂CH₂OMe | Me | H |
| 2-396 | Cl | Cl | 2 | CH₂CH₂OMe | Me | H |
| 2-397 | Cl | Me | 0 | CH₂CH₂OMe | Me | Me |
| 2-398 | Cl | Me | 1 | CH₂CH₂OMe | Me | Me |
| 2-399 | Cl | Me | 2 | CH₂CH₂OMe | Me | Me |
| 2-400 | Cl | Cl | 0 | CH₂CH₂OMe | Me | Me |
| 2-401 | Cl | Cl | 1 | CH₂CH₂OMe | Me | Me |
| 2-402 | Cl | Cl | 2 | CH₂CH₂OMe | Me | Me |
| 2-403 | Cl | Me | 0 | CH₂CH₂OMe | Me | Cl |
| 2-404 | Cl | Me | 1 | CH₂CH₂OMe | Me | Cl |
| 2-405 | Cl | Me | 2 | CH₂CH₂OMe | Me | Cl |
| 2-406 | Cl | Cl | 0 | CH₂CH₂OMe | Me | Cl |
| 2-407 | Cl | Cl | 1 | CH₂CH₂OMe | Me | Cl |
| 2-408 | Cl | Cl | 2 | CH₂CH₂OMe | Me | Cl |
| 2-409 | Cl | Me | 0 | CH₂CH₂OMe | Me | CF₃ |
| 2-410 | Cl | Me | 1 | CH₂CH₂OMe | Me | CF₃ |
| 2-411 | Cl | Me | 2 | CH₂CH₂OMe | Me | CF₃ |
| 2-412 | Cl | Cl | 0 | CH₂CH₂OMe | Me | CF₃ |
| 2-413 | Cl | Cl | 1 | CH₂CH₂OMe | Me | CF₃ |
| 2-414 | Cl | Cl | 2 | CH₂CH₂OMe | Me | CF₃ |
| 2-415 | Cl | Me | 0 | CH₂CH₂OMe | Me | SO₂Me |
| 2-416 | Cl | Me | 1 | CH₂CH₂OMe | Me | SO₂Me |
| 2-417 | Cl | Me | 2 | CH₂CH₂OMe | Me | SO₂Me |
| 2-418 | Cl | Cl | 0 | CH₂CH₂OMe | Me | SO₂Me |
| 2-419 | Cl | Cl | 1 | CH₂CH₂OMe | Me | SO₂Me |
| 2-420 | Cl | Cl | 2 | CH₂CH₂OMe | Me | SO₂Me |
| 2-421 | SO₂Me | Me | 0 | CH₂CH₂OMe | H | H |
| 2-422 | SO₂Me | Me | 1 | CH₂CH₂OMe | H | H |
| 2-423 | SO₂Me | Me | 2 | CH₂CH₂OMe | H | H |
| 2-424 | SO₂Me | Cl | 0 | CH₂CH₂OMe | H | H |
| 2-425 | SO₂Me | Cl | 1 | CH₂CH₂OMe | H | H |
| 2-426 | SO₂Me | Cl | 2 | CH₂CH₂OMe | H | H |
| 2-427 | SO₂Me | Me | 0 | CH₂CH₂OMe | H | Me |
| 2-428 | SO₂Me | Me | 1 | CH₂CH₂OMe | H | Me |
| 2-429 | SO₂Me | Me | 2 | CH₂CH₂OMe | H | Me |
| 2-430 | SO₂Me | Cl | 0 | CH₂CH₂OMe | H | Me |
| 2-431 | SO₂Me | Cl | 1 | CH₂CH₂OMe | H | Me |
| 2-432 | SO₂Me | Cl | 2 | CH₂CH₂OMe | H | Me |
| 2-433 | SO₂Me | Me | 0 | CH₂CH₂OMe | H | Cl |
| 2-434 | SO₂Me | Me | 1 | CH₂CH₂OMe | H | Cl |
| 2-435 | SO₂Me | Me | 2 | CH₂CH₂OMe | H | Cl |
| 2-436 | SO₂Me | Cl | 0 | CH₂CH₂OMe | H | Cl |
| 2-437 | SO₂Me | Cl | 1 | CH₂CH₂OMe | H | Cl |
| 2-438 | SO₂Me | Cl | 2 | CH₂CH₂OMe | H | Cl |
| 2-439 | SO₂Me | Me | 0 | CH₂CH₂OMe | H | CF₃ |
| 2-440 | SO₂Me | Me | 1 | CH₂CH₂OMe | H | CF₃ |
| 2-441 | SO₂Me | Me | 2 | CH₂CH₂OMe | H | CF₃ |
| 2-442 | SO₂Me | Cl | 0 | CH₂CH₂OMe | H | CF₃ |
| 2-443 | SO₂Me | Cl | 1 | CH₂CH₂OMe | H | CF₃ |
| 2-444 | SO₂Me | Cl | 2 | CH₂CH₂OMe | H | CF₃ |
| 2-445 | SO₂Me | Me | 0 | CH₂CH₂OMe | H | SO₂Me |
| 2-446 | SO₂Me | Me | 1 | CH₂CH₂OMe | H | SO₂Me |
| 2-447 | SO₂Me | Me | 2 | CH₂CH₂OMe | H | SO₂Me |
| 2-448 | SO₂Me | Cl | 0 | CH₂CH₂OMe | H | SO₂Me |
| 2-449 | SO₂Me | Cl | 1 | CH₂CH₂OMe | H | SO₂Me |
| 2-450 | SO₂Me | Cl | 2 | CH₂CH₂OMe | H | SO₂Me |
| 2-451 | SO₂Me | Me | 0 | CH₂CH₂OMe | Me | H |
| 2-452 | SO₂Me | Me | 1 | CH₂CH₂OMe | Me | H |
| 2-453 | SO₂Me | Me | 2 | CH₂CH₂OMe | Me | H |
| 2-454 | SO₂Me | Cl | 0 | CH₂CH₂OMe | Me | H |
| 2-455 | SO₂Me | Cl | 1 | CH₂CH₂OMe | Me | H |
| 2-456 | SO₂Me | Cl | 2 | CH₂CH₂OMe | Me | H |
| 2-457 | SO₂Me | Me | 0 | CH₂CH₂OMe | Me | Me |
| 2-458 | SO₂Me | Me | 1 | CH₂CH₂OMe | Me | Me |
| 2-459 | SO₂Me | Me | 2 | CH₂CH₂OMe | Me | Me |
| 2-460 | SO₂Me | Cl | 0 | CH₂CH₂OMe | Me | Me |
| 2-461 | SO₂Me | Cl | 1 | CH₂CH₂OMe | Me | Me |
| 2-462 | SO₂Me | Cl | 2 | CH₂CH₂OMe | Me | Me |
| 2-463 | SO₂Me | Me | 0 | CH₂CH₂OMe | Me | Cl |
| 2-464 | SO₂Me | Me | 1 | CH₂CH₂OMe | Me | Cl |
| 2-465 | SO₂Me | Me | 2 | CH₂CH₂OMe | Me | Cl |
| 2-466 | SO₂Me | Cl | 0 | CH₂CH₂OMe | Me | Cl |
| 2-467 | SO₂Me | Cl | 1 | CH₂CH₂OMe | Me | Cl |
| 2-468 | SO₂Me | Cl | 2 | CH₂CH₂OMe | Me | Cl |
| 2-469 | SO₂Me | Me | 0 | CH₂CH₂OMe | Me | CF₃ |
| 2-470 | SO₂Me | Me | 1 | CH₂CH₂OMe | Me | CF₃ |
| 2-471 | SO₂Me | Me | 2 | CH₂CH₂OMe | Me | CF₃ |
| 2-472 | SO₂Me | Cl | 0 | CH₂CH₂OMe | Me | CF₃ |
| 2-473 | SO₂Me | Cl | 1 | CH₂CH₂OMe | Me | CF₃ |
| 2-474 | SO₂Me | Cl | 2 | CH₂CH₂OMe | Me | CF₃ |
| 2-475 | SO₂Me | Me | 0 | CH₂CH₂OMe | Me | SO₂Me |
| 2-476 | SO₂Me | Me | 1 | CH₂CH₂OMe | Me | SO₂Me |
| 2-477 | SO₂Me | Me | 2 | CH₂CH₂OMe | Me | SO₂Me |
| 2-478 | SO₂Me | Cl | 0 | CH₂CH₂OMe | Me | SO₂Me |
| 2-479 | SO₂Me | Cl | 1 | CH₂CH₂OMe | Me | SO₂Me |
| 2-480 | SO₂Me | Cl | 2 | CH₂CH₂OMe | Me | SO₂Me |

TABLE 3
Compounds according to the invention of the general formula (I) in which
R² and R³ each represent hydrogen, and A represents —CH₂CH₂—,
X₁ represents CH, X² represents CR⁷ and X³ represents CR⁸

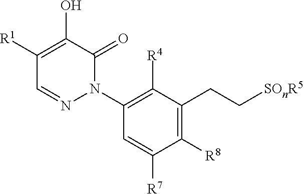

| No. | R¹ | R⁴ | n | R⁵ | R⁷ | R⁸ |
|---|---|---|---|---|---|---|
| 3-1 | Me | Me | 0 | Me | H | H |
| 3-2 | Me | Me | 1 | Me | H | H |
| 3-3 | Me | Me | 2 | Me | H | H |
| 3-4 | Me | Cl | 0 | Me | H | H |
| 3-5 | Me | Cl | 1 | Me | H | H |
| 3-6 | Me | Cl | 2 | Me | H | H |
| 3-7 | Me | Me | 0 | Me | H | Me |
| 3-8 | Me | Me | 1 | Me | H | Me |
| 3-9 | Me | Me | 2 | Me | H | Me |
| 3-10 | Me | Cl | 0 | Me | H | Me |
| 3-11 | Me | Cl | 1 | Me | H | Me |
| 3-12 | Me | Cl | 2 | Me | H | Me |
| 3-13 | Me | Me | 0 | Me | H | Cl |
| 3-14 | Me | Me | 1 | Me | H | Cl |
| 3-15 | Me | Me | 2 | Me | H | Cl |
| 3-16 | Me | Cl | 0 | Me | H | Cl |
| 3-17 | Me | Cl | 1 | Me | H | Cl |
| 3-18 | Me | Cl | 2 | Me | H | Cl |
| 3-19 | Me | Me | 0 | Me | H | CF₃ |
| 3-20 | Me | Me | 1 | Me | H | CF₃ |
| 3-21 | Me | Me | 2 | Me | H | CF₃ |
| 3-22 | Me | Cl | 0 | Me | H | CF₃ |
| 3-23 | Me | Cl | 1 | Me | H | CF₃ |
| 3-24 | Me | Cl | 2 | Me | H | CF₃ |
| 3-25 | Me | Me | 0 | Me | H | SO₂Me |
| 3-26 | Me | Me | 1 | Me | H | SO₂Me |
| 3-27 | Me | Me | 2 | Me | H | SO₂Me |
| 3-28 | Me | Cl | 0 | Me | H | SO₂Me |
| 3-29 | Me | Cl | 1 | Me | H | SO₂Me |
| 3-30 | Me | Cl | 2 | Me | H | SO₂Me |
| 3-31 | Me | Me | 0 | Me | Me | H |
| 3-32 | Me | Me | 1 | Me | Me | H |
| 3-33 | Me | Me | 2 | Me | Me | H |
| 3-34 | Me | Cl | 0 | Me | Me | H |
| 3-35 | Me | Cl | 1 | Me | Me | H |
| 3-36 | Me | Cl | 2 | Me | Me | H |
| 3-37 | Me | Me | 0 | Me | Me | Me |
| 3-38 | Me | Me | 1 | Me | Me | Me |
| 3-39 | Me | Me | 2 | Me | Me | Me |
| 3-40 | Me | Cl | 0 | Me | Me | Me |
| 3-41 | Me | Cl | 1 | Me | Me | Me |
| 3-42 | Me | Cl | 2 | Me | Me | Me |
| 3-43 | Me | Me | 0 | Me | Me | Cl |
| 3-44 | Me | Me | 1 | Me | Me | Cl |
| 3-45 | Me | Me | 2 | Me | Me | Cl |
| 3-46 | Me | Cl | 0 | Me | Me | Cl |
| 3-47 | Me | Cl | 1 | Me | Me | Cl |
| 3-48 | Me | Cl | 2 | Me | Me | Cl |
| 3-49 | Me | Me | 0 | Me | Me | CF₃ |
| 3-50 | Me | Me | 1 | Me | Me | CF₃ |
| 3-51 | Me | Me | 2 | Me | Me | CF₃ |
| 3-52 | Me | Cl | 0 | Me | Me | CF₃ |
| 3-53 | Me | Cl | 1 | Me | Me | CF₃ |
| 3-54 | Me | Cl | 2 | Me | Me | CF₃ |
| 3-55 | Me | Me | 0 | Me | Me | SO₂Me |
| 3-56 | Me | Me | 1 | Me | Me | SO₂Me |
| 3-57 | Me | Me | 2 | Me | Me | SO₂Me |
| 3-58 | Me | Cl | 0 | Me | Me | SO₂Me |
| 3-59 | Me | Cl | 1 | Me | Me | SO₂Me |
| 3-60 | Me | Cl | 2 | Me | Me | SO₂Me |
| 3-61 | c-Pr | Me | 0 | Me | H | H |
| 3-62 | c-Pr | Me | 1 | Me | H | H |
| 3-63 | c-Pr | Me | 2 | Me | H | H |
| 3-64 | c-Pr | Cl | 0 | Me | H | H |
| 3-65 | c-Pr | Cl | 1 | Me | H | H |
| 3-66 | c-Pr | Cl | 2 | Me | H | H |
| 3-67 | c-Pr | Me | 0 | Me | H | Me |
| 3-68 | c-Pr | Me | 1 | Me | H | Me |
| 3-69 | c-Pr | Me | 2 | Me | H | Me |
| 3-70 | c-Pr | Cl | 0 | Me | H | Me |
| 3-71 | c-Pr | Cl | 1 | Me | H | Me |
| 3-72 | c-Pr | Cl | 2 | Me | H | Me |
| 3-73 | c-Pr | Me | 0 | Me | H | Cl |
| 3-74 | c-Pr | Me | 1 | Me | H | Cl |
| 3-75 | c-Pr | Me | 2 | Me | H | Cl |
| 3-76 | c-Pr | Cl | 0 | Me | H | Cl |
| 3-77 | c-Pr | Cl | 1 | Me | H | Cl |
| 3-78 | c-Pr | Cl | 2 | Me | H | Cl |
| 3-79 | c-Pr | Me | 0 | Me | H | CF₃ |
| 3-80 | c-Pr | Me | 1 | Me | H | CF₃ |
| 3-81 | c-Pr | Me | 2 | Me | H | CF₃ |
| 3-82 | c-Pr | Cl | 0 | Me | H | CF₃ |
| 3-83 | c-Pr | Cl | 1 | Me | H | CF₃ |
| 3-84 | c-Pr | Cl | 2 | Me | H | CF₃ |
| 3-85 | c-Pr | Me | 0 | Me | H | SO₂Me |
| 3-86 | c-Pr | Me | 1 | Me | H | SO₂Me |
| 3-87 | c-Pr | Me | 2 | Me | H | SO₂Me |
| 3-88 | c-Pr | Cl | 0 | Me | H | SO₂Me |
| 3-89 | c-Pr | Cl | 1 | Me | H | SO₂Me |
| 3-90 | c-Pr | Cl | 2 | Me | H | SO₂Me |
| 3-91 | c-Pr | Me | 0 | Me | Me | H |
| 3-92 | c-Pr | Me | 1 | Me | Me | H |
| 3-93 | c-Pr | Me | 2 | Me | Me | H |
| 3-94 | c-Pr | Cl | 0 | Me | Me | H |
| 3-95 | c-Pr | Cl | 1 | Me | Me | H |
| 3-96 | c-Pr | Cl | 2 | Me | Me | H |
| 3-97 | c-Pr | Me | 0 | Me | Me | Me |
| 3-98 | c-Pr | Me | 1 | Me | Me | Me |
| 3-99 | c-Pr | Me | 2 | Me | Me | Me |
| 3-100 | c-Pr | Cl | 0 | Me | Me | Me |
| 3-101 | c-Pr | Cl | 1 | Me | Me | Me |
| 3-102 | c-Pr | Cl | 2 | Me | Me | Me |
| 3-103 | c-Pr | Me | 0 | Me | Me | Cl |
| 3-104 | c-Pr | Me | 1 | Me | Me | Cl |
| 3-105 | c-Pr | Me | 2 | Me | Me | Cl |
| 3-106 | c-Pr | Cl | 0 | Me | Me | Cl |
| 3-107 | c-Pr | Cl | 1 | Me | Me | Cl |
| 3-108 | c-Pr | Cl | 2 | Me | Me | Cl |
| 3-109 | c-Pr | Me | 0 | Me | Me | CF₃ |
| 3-110 | c-Pr | Me | 1 | Me | Me | CF₃ |
| 3-111 | c-Pr | Me | 2 | Me | Me | CF₃ |
| 3-112 | c-Pr | Cl | 0 | Me | Me | CF₃ |
| 3-113 | c-Pr | Cl | 1 | Me | Me | CF₃ |
| 3-114 | c-Pr | Cl | 2 | Me | Me | CF₃ |
| 3-115 | c-Pr | Me | 0 | Me | Me | SO₂Me |
| 3-116 | c-Pr | Me | 1 | Me | Me | SO₂Me |
| 3-117 | c-Pr | Me | 2 | Me | Me | SO₂Me |
| 3-118 | c-Pr | Cl | 0 | Me | Me | SO₂Me |
| 3-119 | c-Pr | Cl | 1 | Me | Me | SO₂Me |
| 3-120 | c-Pr | Cl | 2 | Me | Me | SO₂Me |
| 3-121 | Cl | Me | 0 | Me | H | H |
| 3-122 | Cl | Me | 1 | Me | H | H |
| 3-123 | Cl | Me | 2 | Me | H | H |
| 3-124 | Cl | Cl | 0 | Me | H | H |
| 3-125 | Cl | Cl | 1 | Me | H | H |
| 3-126 | Cl | Cl | 2 | Me | H | H |
| 3-127 | Cl | Me | 0 | Me | H | Me |
| 3-128 | Cl | Me | 1 | Me | H | Me |
| 3-129 | Cl | Me | 2 | Me | H | Me |
| 3-130 | Cl | Cl | 0 | Me | H | Me |
| 3-131 | Cl | Cl | 1 | Me | H | Me |
| 3-132 | Cl | Cl | 2 | Me | H | Me |
| 3-133 | Cl | Me | 0 | Me | H | Cl |
| 3-134 | Cl | Me | 1 | Me | H | Cl |
| 3-135 | Cl | Me | 2 | Me | H | Cl |
| 3-136 | Cl | Cl | 0 | Me | H | Cl |
| 3-137 | Cl | Cl | 1 | Me | H | Cl |
| 3-138 | Cl | Cl | 2 | Me | H | Cl |
| 3-139 | Cl | Me | 0 | Me | H | CF₃ |
| 3-140 | Cl | Me | 1 | Me | H | CF₃ |
| 3-141 | Cl | Me | 2 | Me | H | CF₃ |
| 3-142 | Cl | Cl | 0 | Me | H | CF₃ |
| 3-143 | Cl | Cl | 1 | Me | H | CF₃ |
| 3-144 | Cl | Cl | 2 | Me | H | CF₃ |
| 3-145 | Cl | Me | 0 | Me | H | SO₂Me |
| 3-146 | Cl | Me | 1 | Me | H | SO₂Me |
| 3-147 | Cl | Me | 2 | Me | H | SO₂Me |
| 3-148 | Cl | Cl | 0 | Me | H | SO₂Me |
| 3-149 | Cl | Cl | 1 | Me | H | SO₂Me |
| 3-150 | Cl | Cl | 2 | Me | H | SO₂Me |
| 3-151 | Cl | Me | 0 | Me | Me | H |
| 3-152 | Cl | Me | 1 | Me | Me | H |
| 3-153 | Cl | Me | 2 | Me | Me | H |
| 3-154 | Cl | Cl | 0 | Me | Me | H |
| 3-155 | Cl | Cl | 1 | Me | Me | H |
| 3-156 | Cl | Cl | 2 | Me | Me | H |
| 3-157 | Cl | Me | 0 | Me | Me | Me |
| 3-158 | Cl | Me | 1 | Me | Me | Me |
| 3-159 | Cl | Me | 2 | Me | Me | Me |
| 3-160 | Cl | Cl | 0 | Me | Me | Me |
| 3-161 | Cl | Cl | 1 | Me | Me | Me |
| 3-162 | Cl | Cl | 2 | Me | Me | Me |
| 3-163 | Cl | Me | 0 | Me | Me | Cl |
| 3-164 | Cl | Me | 1 | Me | Me | Cl |
| 3-165 | Cl | Me | 2 | Me | Me | Cl |
| 3-166 | Cl | Cl | 0 | Me | Me | Cl |
| 3-167 | Cl | Cl | 1 | Me | Me | Cl |
| 3-168 | Cl | Cl | 2 | Me | Me | Cl |
| 3-169 | Cl | Me | 0 | Me | Me | CF₃ |
| 3-170 | Cl | Me | 1 | Me | Me | CF₃ |
| 3-171 | Cl | Me | 2 | Me | Me | CF₃ |
| 3-172 | Cl | Cl | 0 | Me | Me | CF₃ |
| 3-173 | Cl | Cl | 1 | Me | Me | CF₃ |
| 3-174 | Cl | Cl | 2 | Me | Me | CF₃ |
| 3-175 | Cl | Me | 0 | Me | Me | SO₂Me |
| 3-176 | Cl | Me | 1 | Me | Me | SO₂Me |
| 3-177 | Cl | Me | 2 | Me | Me | SO₂Me |
| 3-178 | Cl | Cl | 0 | Me | Me | SO₂Me |
| 3-179 | Cl | Cl | 1 | Me | Me | SO₂Me |
| 3-180 | Cl | Cl | 2 | Me | Me | SO₂Me |
| 3-181 | SO₂Me | Me | 0 | Me | H | H |
| 3-182 | SO₂Me | Me | 1 | Me | H | H |
| 3-183 | SO₂Me | Me | 2 | Me | H | H |
| 3-184 | SO₂Me | Cl | 0 | Me | H | H |
| 3-185 | SO₂Me | Cl | 1 | Me | H | H |
| 3-186 | SO₂Me | Cl | 2 | Me | H | H |
| 3-187 | SO₂Me | Me | 0 | Me | H | Me |
| 3-188 | SO₂Me | Me | 1 | Me | H | Me |
| 3-189 | SO₂Me | Me | 2 | Me | H | Me |
| 3-190 | SO₂Me | Cl | 0 | Me | H | Me |
| 3-191 | SO₂Me | Cl | 1 | Me | H | Me |
| 3-192 | SO₂Me | Cl | 2 | Me | H | Me |
| 3-193 | SO₂Me | Me | 0 | Me | H | Cl |
| 3-194 | SO₂Me | Me | 1 | Me | H | Cl |
| 3-195 | SO₂Me | Me | 2 | Me | H | Cl |
| 3-196 | SO₂Me | Cl | 0 | Me | H | Cl |
| 3-197 | SO₂Me | Cl | 1 | Me | H | Cl |
| 3-198 | SO₂Me | Cl | 2 | Me | H | Cl |
| 3-199 | SO₂Me | Me | 0 | Me | H | CF₃ |
| 3-200 | SO₂Me | Me | 1 | Me | H | CF₃ |
| 3-201 | SO₂Me | Me | 2 | Me | H | CF₃ |
| 3-202 | SO₂Me | Cl | 0 | Me | H | CF₃ |
| 3-203 | SO₂Me | Cl | 1 | Me | H | CF₃ |
| 3-204 | SO₂Me | Cl | 2 | Me | H | CF₃ |
| 3-205 | SO₂Me | Me | 0 | Me | H | SO₂Me |
| 3-206 | SO₂Me | Me | 1 | Me | H | SO₂Me |
| 3-207 | SO₂Me | Me | 2 | Me | H | SO₂Me |
| 3-208 | SO₂Me | Cl | 0 | Me | H | SO₂Me |
| 3-209 | SO₂Me | Cl | 1 | Me | H | SO₂Me |
| 3-210 | SO₂Me | Cl | 2 | Me | H | SO₂Me |
| 3-211 | SO₂Me | Me | 0 | Me | Me | H |
| 3-212 | SO₂Me | Me | 1 | Me | Me | H |
| 3-213 | SO₂Me | Me | 2 | Me | Me | H |
| 3-214 | SO₂Me | Cl | 0 | Me | Me | H |
| 3-215 | SO₂Me | Cl | 1 | Me | Me | H |
| 3-216 | SO₂Me | Cl | 2 | Me | Me | H |
| 3-217 | SO₂Me | Me | 0 | Me | Me | Me |
| 3-218 | SO₂Me | Me | 1 | Me | Me | Me |
| 3-219 | SO₂Me | Me | 2 | Me | Me | Me |
| 3-220 | SO₂Me | Cl | 0 | Me | Me | Me |
| 3-221 | SO₂Me | Cl | 1 | Me | Me | Me |
| 3-222 | SO₂Me | Cl | 2 | Me | Me | Me |
| 3-223 | SO₂Me | Me | 0 | Me | Me | Cl |
| 3-224 | SO₂Me | Me | 1 | Me | Me | Cl |
| 3-225 | SO₂Me | Me | 2 | Me | Me | Cl |
| 3-226 | SO₂Me | Cl | 0 | Me | Me | Cl |
| 3-227 | SO₂Me | Cl | 1 | Me | Me | Cl |
| 3-228 | SO₂Me | Cl | 2 | Me | Me | Cl |
| 3-229 | SO₂Me | Me | 0 | Me | Me | CF₃ |
| 3-230 | SO₂Me | Me | 1 | Me | Me | CF₃ |
| 3-231 | SO₂Me | Me | 2 | Me | Me | CF₃ |
| 3-232 | SO₂Me | Cl | 0 | Me | Me | CF₃ |
| 3-233 | SO₂Me | Cl | 1 | Me | Me | CF₃ |
| 3-234 | SO₂Me | Cl | 2 | Me | Me | CF₃ |
| 3-235 | SO₂Me | Me | 0 | Me | Me | SO₂Me |
| 3-236 | SO₂Me | Me | 1 | Me | Me | SO₂Me |
| 3-237 | SO₂Me | Me | 2 | Me | Me | SO₂Me |
| 3-238 | SO₂Me | Cl | 0 | Me | Me | SO₂Me |
| 3-239 | SO₂Me | Cl | 1 | Me | Me | SO₂Me |
| 3-240 | SO₂Me | Cl | 2 | Me | Me | SO₂Me |
| 3-241 | Me | Me | 0 | CH₂CH₂OMe | H | H |
| 3-242 | Me | Me | 1 | CH₂CH₂OMe | H | H |
| 3-243 | Me | Me | 2 | CH₂CH₂OMe | H | H |
| 3-244 | Me | Cl | 0 | CH₂CH₂OMe | H | H |
| 3-245 | Me | Cl | 1 | CH₂CH₂OMe | H | H |
| 3-246 | Me | Cl | 2 | CH₂CH₂OMe | H | H |
| 3-247 | Me | Me | 0 | CH₂CH₂OMe | H | Me |
| 3-248 | Me | Me | 1 | CH₂CH₂OMe | H | Me |
| 3-249 | Me | Me | 2 | CH₂CH₂OMe | H | Me |
| 3-250 | Me | Cl | 0 | CH₂CH₂OMe | H | Me |
| 3-251 | Me | Cl | 1 | CH₂CH₂OMe | H | Me |
| 3-252 | Me | Cl | 2 | CH₂CH₂OMe | H | Me |
| 3-253 | Me | Me | 0 | CH₂CH₂OMe | H | Cl |
| 3-254 | Me | Me | 1 | CH₂CH₂OMe | H | Cl |
| 3-255 | Me | Me | 2 | CH₂CH₂OMe | H | Cl |
| 3-256 | Me | Cl | 0 | CH₂CH₂OMe | H | Cl |
| 3-257 | Me | Cl | 1 | CH₂CH₂OMe | H | Cl |
| 3-258 | Me | Cl | 2 | CH₂CH₂OMe | H | Cl |
| 3-259 | Me | Me | 0 | CH₂CH₂OMe | H | CF₃ |
| 3-260 | Me | Me | 1 | CH₂CH₂OMe | H | CF₃ |
| 3-261 | Me | Me | 2 | CH₂CH₂OMe | H | CF₃ |
| 3-262 | Me | Cl | 0 | CH₂CH₂OMe | H | CF₃ |
| 3-263 | Me | Cl | 1 | CH₂CH₂OMe | H | CF₃ |
| 3-264 | Me | Cl | 2 | CH₂CH₂OMe | H | CF₃ |
| 3-265 | Me | Me | 0 | CH₂CH₂OMe | H | SO₂Me |
| 3-266 | Me | Me | 1 | CH₂CH₂OMe | H | SO₂Me |
| 3-267 | Me | Me | 2 | CH₂CH₂OMe | H | SO₂Me |
| 3-268 | Me | Cl | 0 | CH₂CH₂OMe | H | SO₂Me |
| 3-269 | Me | Cl | 1 | CH₂CH₂OMe | H | SO₂Me |
| 3-270 | Me | Cl | 2 | CH₂CH₂OMe | H | SO₂Me |
| 3-271 | Me | Me | 0 | CH₂CH₂OMe | Me | H |
| 3-272 | Me | Me | 1 | CH₂CH₂OMe | Me | H |
| 3-273 | Me | Me | 2 | CH₂CH₂OMe | Me | H |
| 3-274 | Me | Cl | 0 | CH₂CH₂OMe | Me | H |
| 3-275 | Me | Cl | 1 | CH₂CH₂OMe | Me | H |
| 3-276 | Me | Cl | 2 | CH₂CH₂OMe | Me | H |
| 3-277 | Me | Me | 0 | CH₂CH₂OMe | Me | Me |
| 3-278 | Me | Me | 1 | CH₂CH₂OMe | Me | Me |
| 3-279 | Me | Me | 2 | CH₂CH₂OMe | Me | Me |
| 3-280 | Me | Cl | 0 | CH₂CH₂OMe | Me | Me |
| 3-281 | Me | Cl | 1 | CH₂CH₂OMe | Me | Me |
| 3-282 | Me | Cl | 2 | CH₂CH₂OMe | Me | Me |
| 3-283 | Me | Me | 0 | CH₂CH₂OMe | Me | Cl |
| 3-284 | Me | Me | 1 | CH₂CH₂OMe | Me | Cl |
| 3-285 | Me | Me | 2 | CH₂CH₂OMe | Me | Cl |
| 3-286 | Me | Cl | 0 | CH₂CH₂OMe | Me | Cl |
| 3-287 | Me | Cl | 1 | CH₂CH₂OMe | Me | Cl |
| 3-288 | Me | Cl | 2 | CH₂CH₂OMe | Me | Cl |
| 3-289 | Me | Me | 0 | CH₂CH₂OMe | Me | CF₃ |
| 3-290 | Me | Me | 1 | CH₂CH₂OMe | Me | CF₃ |
| 3-291 | Me | Me | 2 | CH₂CH₂OMe | Me | CF₃ |
| 3-292 | Me | Cl | 0 | CH₂CH₂OMe | Me | CF₃ |
| 3-293 | Me | Cl | 1 | CH₂CH₂OMe | Me | CF₃ |
| 3-294 | Me | Cl | 2 | CH₂CH₂OMe | Me | CF₃ |
| 3-295 | Me | Me | 0 | CH₂CH₂OMe | Me | SO₂Me |
| 3-296 | Me | Me | 1 | CH₂CH₂OMe | Me | SO₂Me |
| 3-297 | Me | Me | 2 | CH₂CH₂OMe | Me | SO₂Me |
| 3-298 | Me | Cl | 0 | CH₂CH₂OMe | Me | SO₂Me |
| 3-299 | Me | Cl | 1 | CH₂CH₂OMe | Me | SO₂Me |
| 3-300 | Me | Cl | 2 | CH₂CH₂OMe | Me | SO₂Me |
| 3-301 | c-Pr | Me | 0 | CH₂CH₂OMe | H | H |
| 3-302 | c-Pr | Me | 1 | CH₂CH₂OMe | H | H |
| 3-303 | c-Pr | Me | 2 | CH₂CH₂OMe | H | H |
| 3-304 | c-Pr | Cl | 0 | CH₂CH₂OMe | H | H |
| 3-305 | c-Pr | Cl | 1 | CH₂CH₂OMe | H | H |
| 3-306 | c-Pr | Cl | 2 | CH₂CH₂OMe | H | H |
| 3-307 | c-Pr | Me | 0 | CH₂CH₂OMe | H | Me |
| 3-308 | c-Pr | Me | 1 | CH₂CH₂OMe | H | Me |
| 3-309 | c-Pr | Me | 2 | CH₂CH₂OMe | H | Me |
| 3-310 | c-Pr | Cl | 0 | CH₂CH₂OMe | H | Me |
| 3-311 | c-Pr | Cl | 1 | CH₂CH₂OMe | H | Me |
| 3-312 | c-Pr | Cl | 2 | CH₂CH₂OMe | H | Me |
| 3-313 | c-Pr | Me | 0 | CH₂CH₂OMe | H | Cl |
| 3-314 | c-Pr | Me | 1 | CH₂CH₂OMe | H | Cl |
| 3-315 | c-Pr | Me | 2 | CH₂CH₂OMe | H | Cl |
| 3-316 | c-Pr | Cl | 0 | CH₂CH₂OMe | H | Cl |
| 3-317 | c-Pr | Cl | 1 | CH₂CH₂OMe | H | Cl |
| 3-318 | c-Pr | Cl | 2 | CH₂CH₂OMe | H | Cl |
| 3-319 | c-Pr | Me | 0 | CH₂CH₂OMe | H | CF₃ |
| 3-320 | c-Pr | Me | 1 | CH₂CH₂OMe | H | CF₃ |
| 3-321 | c-Pr | Me | 2 | CH₂CH₂OMe | H | CF₃ |
| 3-322 | c-Pr | Cl | 0 | CH₂CH₂OMe | H | CF₃ |
| 3-323 | c-Pr | Cl | 1 | CH₂CH₂OMe | H | CF₃ |
| 3-324 | c-Pr | Cl | 2 | CH₂CH₂OMe | H | CF₃ |
| 3-325 | c-Pr | Me | 0 | CH₂CH₂OMe | H | SO₂Me |
| 3-326 | c-Pr | Me | 1 | CH₂CH₂OMe | H | SO₂Me |
| 3-327 | c-Pr | Me | 2 | CH₂CH₂OMe | H | SO₂Me |
| 3-328 | c-Pr | Cl | 0 | CH₂CH₂OMe | H | SO₂Me |
| 3-329 | c-Pr | Cl | 1 | CH₂CH₂OMe | H | SO₂Me |
| 3-330 | c-Pr | Cl | 2 | CH₂CH₂OMe | H | SO₂Me |
| 3-331 | c-Pr | Me | 0 | CH₂CH₂OMe | Me | H |
| 3-332 | c-Pr | Me | 1 | CH₂CH₂OMe | Me | H |
| 3-333 | c-Pr | Me | 2 | CH₂CH₂OMe | Me | H |
| 3-334 | c-Pr | Cl | 0 | CH₂CH₂OMe | Me | H |
| 3-335 | c-Pr | Cl | 1 | CH₂CH₂OMe | Me | H |
| 3-336 | c-Pr | Cl | 2 | CH₂CH₂OMe | Me | H |
| 3-337 | c-Pr | Me | 0 | CH₂CH₂OMe | Me | Me |
| 3-338 | c-Pr | Me | 1 | CH₂CH₂OMe | Me | Me |
| 3-339 | c-Pr | Me | 2 | CH₂CH₂OMe | Me | Me |
| 3-340 | c-Pr | Cl | 0 | CH₂CH₂OMe | Me | Me |
| 3-341 | c-Pr | Cl | 1 | CH₂CH₂OMe | Me | Me |
| 3-342 | c-Pr | Cl | 2 | CH₂CH₂OMe | Me | Me |
| 3-343 | c-Pr | Me | 0 | CH₂CH₂OMe | Me | Cl |
| 3-344 | c-Pr | Me | 1 | CH₂CH₂OMe | Me | Cl |
| 3-345 | c-Pr | Me | 2 | CH₂CH₂OMe | Me | Cl |
| 3-346 | c-Pr | Cl | 0 | CH₂CH₂OMe | Me | Cl |
| 3-347 | c-Pr | Cl | 1 | CH₂CH₂OMe | Me | Cl |
| 3-348 | c-Pr | Cl | 2 | CH₂CH₂OMe | Me | Cl |
| 3-349 | c-Pr | Me | 0 | CH₂CH₂OMe | Me | CF₃ |
| 3-350 | c-Pr | Me | 1 | CH₂CH₂OMe | Me | CF₃ |
| 3-351 | c-Pr | Me | 2 | CH₂CH₂OMe | Me | CF₃ |
| 3-352 | c-Pr | Cl | 0 | CH₂CH₂OMe | Me | CF₃ |
| 3-353 | c-Pr | Cl | 1 | CH₂CH₂OMe | Me | CF₃ |
| 3-354 | c-Pr | Cl | 2 | CH₂CH₂OMe | Me | CF₃ |
| 3-355 | c-Pr | Me | 0 | CH₂CH₂OMe | Me | SO₂Me |
| 3-356 | c-Pr | Me | 1 | CH₂CH₂OMe | Me | SO₂Me |
| 3-357 | c-Pr | Me | 2 | CH₂CH₂OMe | Me | SO₂Me |
| 3-358 | c-Pr | Cl | 0 | CH₂CH₂OMe | Me | SO₂Me |
| 3-359 | c-Pr | Cl | 1 | CH₂CH₂OMe | Me | SO₂Me |
| 3-360 | c-Pr | Cl | 2 | CH₂CH₂OMe | Me | SO₂Me |
| 3-361 | Cl | Me | 0 | CH₂CH₂OMe | H | H |
| 3-362 | Cl | Me | 1 | CH₂CH₂OMe | H | H |
| 3-363 | Cl | Me | 2 | CH₂CH₂OMe | H | H |
| 3-364 | Cl | Cl | 0 | CH₂CH₂OMe | H | H |
| 3-365 | Cl | Cl | 1 | CH₂CH₂OMe | H | H |
| 3-366 | Cl | Cl | 2 | CH₂CH₂OMe | H | H |
| 3-367 | Cl | Me | 0 | CH₂CH₂OMe | H | Me |
| 3-368 | Cl | Me | 1 | CH₂CH₂OMe | H | Me |
| 3-369 | Cl | Me | 2 | CH₂CH₂OMe | H | Me |
| 3-370 | Cl | Cl | 0 | CH₂CH₂OMe | H | Me |
| 3-371 | Cl | Cl | 1 | CH₂CH₂OMe | H | Me |
| 3-372 | Cl | Cl | 2 | CH₂CH₂OMe | H | Me |
| 3-373 | Cl | Me | 0 | CH₂CH₂OMe | H | Cl |
| 3-374 | Cl | Me | 1 | CH₂CH₂OMe | H | Cl |
| 3-375 | Cl | Me | 2 | CH₂CH₂OMe | H | Cl |
| 3-376 | Cl | Cl | 0 | CH₂CH₂OMe | H | Cl |
| 3-377 | Cl | Cl | 1 | CH₂CH₂OMe | H | Cl |
| 3-378 | Cl | Cl | 2 | CH₂CH₂OMe | H | Cl |
| 3-379 | Cl | Me | 0 | CH₂CH₂OMe | H | CF₃ |
| 3-380 | Cl | Me | 1 | CH₂CH₂OMe | H | CF₃ |
| 3-381 | Cl | Me | 2 | CH₂CH₂OMe | H | CF₃ |
| 3-382 | Cl | Cl | 0 | CH₂CH₂OMe | H | CF₃ |
| 3-383 | Cl | Cl | 1 | CH₂CH₂OMe | H | CF₃ |
| 3-384 | Cl | Cl | 2 | CH₂CH₂OMe | H | CF₃ |
| 3-385 | Cl | Me | 0 | CH₂CH₂OMe | H | SO₂Me |
| 3-386 | Cl | Me | 1 | CH₂CH₂OMe | H | SO₂Me |
| 3-387 | Cl | Me | 2 | CH₂CH₂OMe | H | SO₂Me |
| 3-388 | Cl | Cl | 0 | CH₂CH₂OMe | H | SO₂Me |
| 3-389 | Cl | Cl | 1 | CH₂CH₂OMe | H | SO₂Me |
| 3-390 | Cl | Cl | 2 | CH₂CH₂OMe | H | SO₂Me |
| 3-391 | Cl | Me | 0 | CH₂CH₂OMe | Me | H |
| 3-392 | Cl | Me | 1 | CH₂CH₂OMe | Me | H |
| 3-393 | Cl | Me | 2 | CH₂CH₂OMe | Me | H |
| 3-394 | Cl | Cl | 0 | CH₂CH₂OMe | Me | H |
| 3-395 | Cl | Cl | 1 | CH₂CH₂OMe | Me | H |
| 3-396 | Cl | Cl | 2 | CH₂CH₂OMe | Me | H |
| 3-397 | Cl | Me | 0 | CH₂CH₂OMe | Me | Me |
| 3-398 | Cl | Me | 1 | CH₂CH₂OMe | Me | Me |
| 3-399 | Cl | Me | 2 | CH₂CH₂OMe | Me | Me |
| 3-400 | Cl | Cl | 0 | CH₂CH₂OMe | Me | Me |
| 3-401 | Cl | Cl | 1 | CH₂CH₂OMe | Me | Me |
| 3-402 | Cl | Cl | 2 | CH₂CH₂OMe | Me | Me |
| 3-403 | Cl | Me | 0 | CH₂CH₂OMe | Me | Cl |
| 3-404 | Cl | Me | 1 | CH₂CH₂OMe | Me | Cl |
| 3-405 | Cl | Me | 2 | CH₂CH₂OMe | Me | Cl |
| 3-406 | Cl | Cl | 0 | CH₂CH₂OMe | Me | Cl |
| 3-407 | Cl | Cl | 1 | CH₂CH₂OMe | Me | Cl |
| 3-408 | Cl | Cl | 2 | CH₂CH₂OMe | Me | Cl |
| 3-409 | Cl | Me | 0 | CH₂CH₂OMe | Me | CF₃ |
| 3-410 | Cl | Me | 1 | CH₂CH₂OMe | Me | CF₃ |
| 3-411 | Cl | Me | 2 | CH₂CH₂OMe | Me | CF₃ |
| 3-412 | Cl | Cl | 0 | CH₂CH₂OMe | Me | CF₃ |
| 3-413 | Cl | Cl | 1 | CH₂CH₂OMe | Me | CF₃ |
| 3-414 | Cl | Cl | 2 | CH₂CH₂OMe | Me | CF₃ |
| 3-415 | Cl | Me | 0 | CH₂CH₂OMe | Me | SO₂Me |
| 3-416 | Cl | Me | 1 | CH₂CH₂OMe | Me | SO₂Me |
| 3-417 | Cl | Me | 2 | CH₂CH₂OMe | Me | SO₂Me |
| 3-418 | Cl | Cl | 0 | CH₂CH₂OMe | Me | SO₂Me |
| 3-419 | Cl | Cl | 1 | CH₂CH₂OMe | Me | SO₂Me |
| 3-420 | Cl | Cl | 2 | CH₂CH₂OMe | Me | SO₂Me |
| 3-421 | SO₂Me | Me | 0 | CH₂CH₂OMe | H | H |
| 3-422 | SO₂Me | Me | 1 | CH₂CH₂OMe | H | H |
| 3-423 | SO₂Me | Me | 2 | CH₂CH₂OMe | H | H |
| 3-424 | SO₂Me | Cl | 0 | CH₂CH₂OMe | H | H |
| 3-425 | SO₂Me | Cl | 1 | CH₂CH₂OMe | H | H |
| 3-426 | SO₂Me | Cl | 2 | CH₂CH₂OMe | H | H |
| 3-427 | SO₂Me | Me | 0 | CH₂CH₂OMe | H | Me |
| 3-428 | SO₂Me | Me | 1 | CH₂CH₂OMe | H | Me |
| 3-429 | SO₂Me | Me | 2 | CH₂CH₂OMe | H | Me |
| 3-430 | SO₂Me | Cl | 0 | CH₂CH₂OMe | H | Me |
| 3-431 | SO₂Me | Cl | 1 | CH₂CH₂OMe | H | Me |
| 3-432 | SO₂Me | Cl | 2 | CH₂CH₂OMe | H | Me |
| 3-433 | SO₂Me | Me | 0 | CH₂CH₂OMe | H | Cl |
| 3-434 | SO₂Me | Me | 1 | CH₂CH₂OMe | H | Cl |
| 3-435 | SO₂Me | Me | 2 | CH₂CH₂OMe | H | Cl |
| 3-436 | SO₂Me | Cl | 0 | CH₂CH₂OMe | H | Cl |
| 3-437 | SO₂Me | Cl | 1 | CH₂CH₂OMe | H | Cl |
| 3-438 | SO₂Me | Cl | 2 | CH₂CH₂OMe | H | Cl |
| 3-439 | SO₂Me | Me | 0 | CH₂CH₂OMe | H | CF₃ |
| 3-440 | SO₂Me | Me | 1 | CH₂CH₂OMe | H | CF₃ |
| 3-441 | SO₂Me | Me | 2 | CH₂CH₂OMe | H | CF₃ |
| 3-442 | SO₂Me | Cl | 0 | CH₂CH₂OMe | H | CF₃ |
| 3-443 | SO₂Me | Cl | 1 | CH₂CH₂OMe | H | CF₃ |
| 3-444 | SO₂Me | Cl | 2 | CH₂CH₂OMe | H | CF₃ |
| 3-445 | SO₂Me | Me | 0 | CH₂CH₂OMe | H | SO₂Me |
| 3-446 | SO₂Me | Me | 1 | CH₂CH₂OMe | H | SO₂Me |
| 3-447 | SO₂Me | Me | 2 | CH₂CH₂OMe | H | SO₂Me |
| 3-448 | SO₂Me | Cl | 0 | CH₂CH₂OMe | H | SO₂Me |
| 3-449 | SO₂Me | Cl | 1 | CH₂CH₂OMe | H | SO₂Me |
| 3-450 | SO₂Me | Cl | 2 | CH₂CH₂OMe | H | SO₂Me |
| 3-451 | SO₂Me | Me | 0 | CH₂CH₂OMe | Me | H |
| 3-452 | SO₂Me | Me | 1 | CH₂CH₂OMe | Me | H |
| 3-453 | SO₂Me | Me | 2 | CH₂CH₂OMe | Me | H |
| 3-454 | SO₂Me | Cl | 0 | CH₂CH₂OMe | Me | H |
| 3-455 | SO₂Me | Cl | 1 | CH₂CH₂OMe | Me | H |
| 3-456 | SO₂Me | Cl | 2 | CH₂CH₂OMe | Me | H |
| 3-457 | SO₂Me | Me | 0 | CH₂CH₂OMe | Me | Me |
| 3-458 | SO₂Me | Me | 1 | CH₂CH₂OMe | Me | Me |
| 3-459 | SO₂Me | Me | 2 | CH₂CH₂OMe | Me | Me |
| 3-460 | SO₂Me | Cl | 0 | CH₂CH₂OMe | Me | Me |
| 3-461 | SO₂Me | Cl | 1 | CH₂CH₂OMe | Me | Me |
| 3-462 | SO₂Me | Cl | 2 | CH₂CH₂OMe | Me | Me |
| 3-463 | SO₂Me | Me | 0 | CH₂CH₂OMe | Me | Cl |
| 3-464 | SO₂Me | Me | 1 | CH₂CH₂OMe | Me | Cl |
| 3-465 | S0₂Me | Me | 2 | CH₂CH₂OMe | Me | Cl |
| 3-466 | SO₂Me | Cl | 0 | CH₂CH₂OMe | Me | Cl |
| 3-467 | SO₂Me | Cl | 1 | CH₂CH₂OMe | Me | Cl |
| 3-468 | SO₂Me | Cl | 2 | CH₂CH₂OMe | Me | Cl |
| 3-469 | SO₂Me | Me | 0 | CH₂CH₂OMe | Me | CF₃ |
| 3-470 | SO₂Me | Me | 1 | CH₂CH₂OMe | Me | CF₃ |
| 3-471 | SO₂Me | Me | 2 | CH₂CH₂OMe | Me | CF₃ |
| 3-472 | SO₂Me | Cl | 0 | CH₂CH₂OMe | Me | CF₃ |
| 3-473 | SO₂Me | Cl | 1 | CH₂CH₂OMe | Me | CF₃ |
| 3-474 | SO₂Me | Cl | 2 | CH₂CH₂OMe | Me | CF₃ |
| 3-475 | SO₂Me | Me | 0 | CH₂CH₂OMe | Me | SO₂Me |
| 3-476 | SO₂Me | Me | 1 | CH₂CH₂OMe | Me | SO₂Me |
| 3-477 | SO₂Me | Me | 2 | CH₂CH₂OMe | Me | SO₂Me |
| 3-478 | SO₂Me | Cl | 0 | CH₂CH₂OMe | Me | SO₂Me |
| 3-479 | SO₂Me | Cl | 1 | CH₂CH₂OMe | Me | SO₂Me |
| 3-480 | SO₂Me | Cl | 2 | CH₂CH₂OMe | Me | SO₂Me |

TABLE 4
Compounds according to the invention of the general formula (I) in
which R² represents hydrogen and R³ represents acetyl, A represents
a direct bond, X¹ and X² each represent CH and X³ represents CR⁸

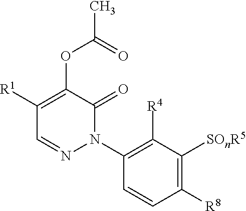

| No. | R¹ | R⁴ | n | R⁵ | R⁸ |
|---|---|---|---|---|---|
| 4-1 | Me | Me | 0 | CH₂—c-Pr | CF₃ |
| 4-2 | Me | Me | 1 | CH₂—c-Pr | CF₃ |
| 4-3 | Me | Me | 2 | CH₂—c-Pr | CF₃ |
| 4-4 | Me | Cl | 0 | CH₂—c-Pr | CF₃ |
| 4-5 | Me | Cl | 1 | CH₂—c-Pr | CF₃ |
| 4-6 | Me | Cl | 2 | CH₂—c-Pr | CF₃ |
| 4-7 | Me | Me | 0 | CH₂—c-Pr | SO₂Me |
| 4-8 | Me | Me | 1 | CH₂—c-Pr | SO₂Me |
| 4-9 | Me | Me | 2 | CH₂—c-Pr | SO₂Me |
| 4-10 | Me | Cl | 0 | CH₂—c-Pr | SO₂Me |
| 4-11 | Me | Cl | 1 | CH₂—c-Pr | SO₂Me |
| 4-12 | Me | Cl | 2 | CH₂—c-Pr | SO₂Me |
| 4-13 | Cl | Me | 0 | CH₂—c-Pr | CF₃ |
| 4-14 | Cl | Me | 1 | CH₂—c-Pr | CF₃ |
| 4-15 | Cl | Me | 2 | CH₂—c-Pr | CF₃ |
| 4-16 | Cl | Cl | 0 | CH₂—c-Pr | CF₃ |
| 4-17 | Cl | Cl | 1 | CH₂—c-Pr | CF₃ |
| 4-18 | Cl | Cl | 2 | CH₂—c-Pr | CF₃ |
| 4-19 | Cl | Me | 0 | CH₂—c-Pr | SO₂Me |
| 4-20 | Cl | Me | 1 | CH₂—c-Pr | SO₂Me |
| 4-21 | Cl | Me | 2 | CH₂—c-Pr | SO₂Me |
| 4-22 | Cl | Cl | 0 | CH₂—c-Pr | SO₂Me |
| 4-23 | Cl | Cl | 1 | CH₂—c-Pr | SO₂Me |
| 4-24 | Cl | Cl | 2 | CH₂—c-Pr | SO₂Me |
| 4-25 | SO₂Me | Me | 0 | CH₂—c-Pr | CF₃ |
| 4-26 | SO₂Me | Me | 1 | CH₂—c-Pr | CF₃ |
| 4-27 | SO₂Me | Me | 2 | CH₂—c-Pr | CF₃ |
| 4-28 | SO₂Me | Cl | 0 | CH₂—c-Pr | CF₃ |
| 4-29 | SO₂Me | Cl | 1 | CH₂—c-Pr | CF₃ |
| 4-30 | SO₂Me | Cl | 2 | CH₂—c-Pr | CF₃ |
| 4-31 | SO₂Me | Me | 0 | CH₂—c-Pr | SO₂Me |
| 4-32 | SO₂Me | Me | 1 | CH₂—c-Pr | SO₂Me |
| 4-33 | SO₂Me | Me | 2 | CH₂—c-Pr | SO₂Me |
| 4-34 | SO₂Me | Cl | 0 | CH₂—c-Pr | SO₂Me |
| 4-35 | SO₂Me | Cl | 1 | CH₂—c-Pr | SO₂Me |
| 4-36 | SO₂Me | Cl | 2 | CH₂—c-Pr | SO₂Me |
| 4-37 | Me | Me | 0 | CH₂CH₂OMe | CF₃ |
| 4-38 | Me | Me | 1 | CH₂CH₂OMe | CF₃ |
| 4-39 | Me | Me | 2 | CH₂CH₂OMe | CF₃ |
| 4-40 | Me | Cl | 0 | CH₂CH₂OMe | CF₃ |
| 4-41 | Me | Cl | 1 | CH₂CH₂OMe | CF₃ |
| 4-42 | Me | Cl | 2 | CH₂CH₂OMe | CF₃ |
| 4-43 | Me | Me | 0 | CH₂CH₂OMe | SO₂Me |
| 4-44 | Me | Me | 1 | CH₂CH₂OMe | SO₂Me |
| 4-45 | Me | Me | 2 | CH₂CH₂OMe | SO₂Me |
| 4-46 | Me | Cl | 0 | CH₂CH₂OMe | SO₂Me |
| 4-47 | Me | Cl | 1 | CH₂CH₂OMe | SO₂Me |
| 4-48 | Me | Cl | 2 | CH₂CH₂OMe | SO₂Me |
| 4-49 | Cl | Me | 0 | CH₂CH₂OMe | CF₃ |
| 4-50 | Cl | Me | 1 | CH₂CH₂OMe | CF₃ |
| 4-51 | Cl | Me | 2 | CH₂CH₂OMe | CF₃ |
| 4-52 | Cl | Cl | 0 | CH₂CH₂OMe | CF₃ |
| 4-53 | Cl | Cl | 1 | CH₂CH₂OMe | CF₃ |
| 4-54 | Cl | Cl | 2 | CH₂CH₂OMe | CF₃ |
| 4-55 | Cl | Me | 0 | CH₂CH₂OMe | SO₂Me |
| 4-56 | Cl | Me | 1 | CH₂CH₂OMe | SO₂Me |
| 4-57 | Cl | Me | 2 | CH₂CH₂OMe | SO₂Me |
| 4-58 | Cl | Cl | 0 | CH₂CH₂OMe | SO₂Me |
| 4-59 | Cl | Cl | 1 | CH₂CH₂OMe | SO₂Me |
| 4-60 | Cl | Cl | 2 | CH₂CH₂OMe | SO₂Me |
| 4-61 | SO₂Me | Me | 0 | CH₂CH₂OMe | CF₃ |
| 4-62 | SO₂Me | Me | 1 | CH₂CH₂OMe | CF₃ |
| 4-63 | SO₂Me | Me | 2 | CH₂CH₂OMe | CF₃ |
| 4-64 | SO₂Me | Cl | 0 | CH₂CH₂OMe | CF₃ |
| 4-65 | SO₂Me | Cl | 1 | CH₂CH₂OMe | CF₃ |
| 4-66 | SO₂Me | Cl | 2 | CH₂CH₂OMe | CF₃ |
| 4-67 | SO₂Me | Me | 0 | CH₂CH₂OMe | SO₂Me |
| 4-68 | SO₂Me | Me | 1 | CH₂CH₂OMe | SO₂Me |
| 4-69 | SO₂Me | Me | 2 | CH₂CH₂OMe | SO₂Me |
| 4-70 | SO₂Me | Cl | 0 | CH₂CH₂OMe | SO₂Me |
| 4-71 | SO₂Me | Cl | 1 | CH₂CH₂OMe | SO₂Me |
| 4-72 | SO₂Me | Cl | 2 | CH₂CH₂OMe | SO₂Me |

TABLE 5
Compounds according to the invention of the general formula (I) in
which R² represents hydrogen and R³ represents acetyl, A represents
—CH₂—, X¹ and X² each represent CH and X³ represents CR⁸

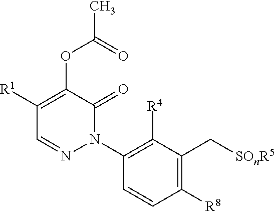

| No. | R¹ | R⁴ | n | R⁵ | R⁸ |
|---|---|---|---|---|---|
| 5-1 | Me | Me | 0 | Me | CF₃ |
| 5-2 | Me | Me | 1 | Me | CF₃ |
| 5-3 | Me | Me | 2 | Me | CF₃ |
| 5-4 | Me | Cl | 0 | Me | CF₃ |
| 5-5 | Me | Cl | 1 | Me | CF₃ |
| 5-6 | Me | Cl | 2 | Me | CF₃ |
| 5-7 | Me | Me | 0 | Me | SO₂Me |
| 5-8 | Me | Me | 1 | Me | SO₂Me |
| 5-9 | Me | Me | 2 | Me | SO₂Me |
| 5-10 | Me | Cl | 0 | Me | SO₂Me |
| 5-11 | Me | Cl | 1 | Me | SO₂Me |
| 5-12 | Me | Cl | 2 | Me | SO₂Me |
| 5-13 | Cl | Me | 0 | Me | CF₃ |
| 5-14 | Cl | Me | 1 | Me | CF₃ |
| 5-15 | Cl | Me | 2 | Me | CF₃ |
| 5-16 | Cl | Cl | 0 | Me | CF₃ |
| 5-17 | Cl | Cl | 1 | Me | CF₃ |
| 5-18 | Cl | Cl | 2 | Me | CF₃ |
| 5-19 | Cl | Me | 0 | Me | SO₂Me |
| 5-20 | Cl | Me | 1 | Me | SO₂Me |
| 5-21 | Cl | Me | 2 | Me | SO₂Me |
| 5-22 | Cl | Cl | 0 | Me | SO₂Me |
| 5-23 | Cl | Cl | 1 | Me | SO₂Me |
| 5-24 | Cl | Cl | 2 | Me | SO₂Me |
| 5-25 | SO₂Me | Me | 0 | Me | CF₃ |
| 5-26 | SO₂Me | Me | 1 | Me | CF₃ |
| 5-27 | SO₂Me | Me | 2 | Me | CF₃ |
| 5-28 | SO₂Me | Cl | 0 | Me | CF₃ |
| 5-29 | SO₂Me | Cl | 1 | Me | CF₃ |
| 5-30 | SO₂Me | Cl | 2 | Me | CF₃ |
| 5-31 | SO₂Me | Me | 0 | Me | SO₂Me |
| 5-32 | SO₂Me | Me | 1 | Me | SO₂Me |
| 5-33 | SO₂Me | Me | 2 | Me | SO₂Me |
| 5-34 | SO₂Me | Cl | 0 | Me | SO₂Me |
| 5-35 | SO₂Me | Cl | 1 | Me | SO₂Me |
| 5-36 | SO₂Me | Cl | 2 | Me | SO₂Me |
| 5-37 | Me | Me | 0 | CH₂CH₂OMe | CF₃ |
| 5-38 | Me | Me | 1 | CH₂CH₂OMe | CF₃ |
| 5-39 | Me | Me | 2 | CH₂CH₂OMe | CF₃ |
| 5-40 | Me | Cl | 0 | CH₂CH₂OMe | CF₃ |
| 5-41 | Me | Cl | 1 | CH₂CH₂OMe | CF₃ |
| 5-42 | Me | Cl | 2 | CH₂CH₂OMe | CF₃ |
| 5-43 | Me | Me | 0 | CH₂CH₂OMe | SO₂Me |
| 5-44 | Me | Me | 1 | CH₂CH₂OMe | SO₂Me |
| 5-45 | Me | Me | 2 | CH₂CH₂OMe | SO₂Me |
| 5-46 | Me | Cl | 0 | CH₂CH₂OMe | SO₂Me |
| 5-47 | Me | Cl | 1 | CH₂CH₂OMe | SO₂Me |
| 5-48 | Me | Cl | 2 | CH₂CH₂OMe | SO₂Me |
| 5-49 | Cl | Me | 0 | CH₂CH₂OMe | CF₃ |
| 5-50 | Cl | Me | 1 | CH₂CH₂OMe | CF₃ |
| 5-51 | Cl | Me | 2 | CH₂CH₂OMe | CF₃ |
| 5-52 | Cl | Cl | 0 | CH₂CH₂OMe | CF₃ |
| 5-53 | Cl | Cl | 1 | CH₂CH₂OMe | CF₃ |
| 5-54 | Cl | Cl | 2 | CH₂CH₂OMe | CF₃ |
| 5-55 | Cl | Me | 0 | CH₂CH₂OMe | SO₂Me |
| 5-56 | Cl | Me | 1 | CH₂CH₂OMe | SO₂Me |
| 5-57 | Cl | Me | 2 | CH₂CH₂OMe | SO₂Me |
| 5-58 | Cl | Cl | 0 | CH₂CH₂OMe | SO₂Me |
| 5-59 | Cl | Cl | 1 | CH₂CH₂OMe | SO₂Me |
| 5-60 | Cl | Cl | 2 | CH₂CH₂OMe | SO₂Me |
| 5-61 | SO₂Me | Me | 0 | CH₂CH₂OMe | CF₃ |
| 5-62 | SO₂Me | Me | 1 | CH₂CH₂OMe | CF₃ |
| 5-63 | SO₂Me | Me | 2 | CH₂CH₂OMe | CF₃ |
| 5-64 | SO₂Me | Cl | 0 | CH₂CH₂OMe | CF₃ |
| 5-65 | SO₂Me | Cl | 1 | CH₂CH₂OMe | CF₃ |
| 5-66 | SO₂Me | Cl | 2 | CH₂CH₂OMe | CF₃ |
| 5-67 | SO₂Me | Me | 0 | CH₂CH₂OMe | SO₂Me |
| 5-68 | SO₂Me | Me | 1 | CH₂CH₂OMe | SO₂Me |
| 5-69 | SO₂Me | Me | 2 | CH₂CH₂OMe | SO₂Me |
| 5-70 | SO₂Me | Cl | 0 | CH₂CH₂OMe | SO₂Me |
| 5-71 | SO₂Me | Cl | 1 | CH₂CH₂OMe | SO₂Me |
| 5-72 | SO₂Me | Cl | 2 | CH₂CH₂OMe | SO₂Me |

TABLE 6
Compounds according to the invention of the general formula (I) in
which R² represents hydrogen and R³ represents acetyl, A represents
—CH₂CH₂—, X¹ and X² each represent CH and X³ represents CR⁸

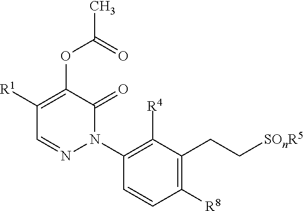

| No. | R¹ | R⁴ | n | R⁵ | R⁸ |
|---|---|---|---|---|---|
| 6-1 | Me | Me | 0 | Me | CF₃ |
| 6-2 | Me | Me | 1 | Me | CF₃ |
| 6-3 | Me | Me | 2 | Me | CF₃ |
| 6-4 | Me | Cl | 0 | Me | CF₃ |
| 6-5 | Me | Cl | 1 | Me | CF₃ |
| 6-6 | Me | Cl | 2 | Me | CF₃ |
| 6-7 | Me | Me | 0 | Me | SO₂Me |
| 6-8 | Me | Me | 1 | Me | SO₂Me |
| 6-9 | Me | Me | 2 | Me | SO₂Me |
| 6-10 | Me | Cl | 0 | Me | SO₂Me |
| 6-11 | Me | Cl | 1 | Me | SO₂Me |
| 6-12 | Me | Cl | 2 | Me | SO₂Me |
| 6-13 | Cl | Me | 0 | Me | CF₃ |
| 6-14 | Cl | Me | 1 | Me | CF₃ |
| 6-15 | Cl | Me | 2 | Me | CF₃ |
| 6-16 | Cl | Cl | 0 | Me | CF₃ |
| 6-17 | Cl | Cl | 1 | Me | CF₃ |
| 6-18 | Cl | Cl | 2 | Me | CF₃ |
| 6-19 | Cl | Me | 0 | Me | SO₂Me |
| 6-20 | Cl | Me | 1 | Me | SO₂Me |
| 6-21 | Cl | Me | 2 | Me | SO₂Me |
| 6-22 | Cl | Cl | 0 | Me | SO₂Me |
| 6-23 | Cl | Cl | 1 | Me | SO₂Me |
| 6-24 | Cl | Cl | 2 | Me | SO₂Me |
| 6-25 | SO₂Me | Me | 0 | Me | CF₃ |
| 6-26 | SO₂Me | Me | 1 | Me | CF₃ |
| 6-27 | SO₂Me | Me | 2 | Me | CF₃ |
| 6-28 | SO₂Me | Cl | 0 | Me | CF₃ |
| 6-29 | SO₂Me | Cl | 1 | Me | CF₃ |
| 6-30 | SO₂Me | Cl | 2 | Me | CF₃ |
| 6-31 | SO₂Me | Me | 0 | Me | SO₂Me |
| 6-32 | SO₂Me | Me | 1 | Me | SO₂Me |
| 6-33 | SO₂Me | Me | 2 | Me | SO₂Me |
| 6-34 | SO₂Me | Cl | 0 | Me | SO₂Me |
| 6-35 | SO₂Me | Cl | 1 | Me | SO₂Me |
| 6-36 | SO₂Me | Cl | 2 | Me | SO₂Me |
| 6-37 | Me | Me | 0 | CH₂CH₂OMe | CF₃ |
| 6-38 | Me | Me | 1 | CH₂CH₂OMe | CF₃ |
| 6-39 | Me | Me | 2 | CH₂CH₂OMe | CF₃ |
| 6-40 | Me | Cl | 0 | CH₂CH₂OMe | CF₃ |
| 6-41 | Me | Cl | 1 | CH₂CH₂OMe | CF₃ |
| 6-42 | Me | Cl | 2 | CH₂CH₂OMe | CF₃ |
| 6-43 | Me | Me | 0 | CH₂CH₂OMe | SO₂Me |
| 6-44 | Me | Me | 1 | CH₂CH₂OMe | SO₂Me |
| 6-45 | Me | Me | 2 | CH₂CH₂OMe | SO₂Me |
| 6-46 | Me | Cl | 0 | CH₂CH₂OMe | SO₂Me |
| 6-47 | Me | Cl | 1 | CH₂CH₂OMe | SO₂Me |
| 6-48 | Me | Cl | 2 | CH₂CH₂OMe | SO₂Me |
| 6-49 | Cl | Me | 0 | CH₂CH₂OMe | CF₃ |
| 6-50 | Cl | Me | 1 | CH₂CH₂OMe | CF₃ |
| 6-51 | Cl | Me | 2 | CH₂CH₂OMe | CF₃ |
| 6-52 | Cl | Cl | 0 | CH₂CH₂OMe | CF₃ |
| 6-53 | Cl | Cl | 1 | CH₂CH₂OMe | CF₃ |
| 6-54 | Cl | Cl | 2 | CH₂CH₂OMe | CF₃ |
| 6-55 | Cl | Me | 0 | CH₂CH₂OMe | SO₂Me |
| 6-56 | Cl | Me | 1 | CH₂CH₂OMe | SO₂Me |
| 6-57 | Cl | Me | 2 | CH₂CH₂OMe | SO₂Me |
| 6-58 | Cl | Cl | 0 | CH₂CH₂OMe | SO₂Me |
| 6-59 | Cl | Cl | 1 | CH₂CH₂OMe | SO₂Me |
| 6-60 | Cl | Cl | 2 | CH₂CH₂OMe | SO₂Me |
| 6-61 | SO₂Me | Me | 0 | CH₂CH₂OMe | CF₃ |
| 6-62 | SO₂Me | Me | 1 | CH₂CH₂OMe | CF₃ |
| 6-63 | SO₂Me | Me | 2 | CH₂CH₂OMe | CF₃ |
| 6-64 | SO₂Me | Cl | 0 | CH₂CH₂OMe | SO₂Me |
| 6-65 | SO₂Me | Cl | 1 | CH₂CH₂OMe | SO₂Me |
| 6-66 | SO₂Me | Cl | 2 | CH₂CH₂OMe | SO₂Me |
| 6-67 | SO₂Me | Me | 0 | CH₂CH₂OMe | SO₂Me |
| 6-68 | SO₂Me | Me | 1 | CH₂CH₂OMe | SO₂Me |
| 6-69 | SO₂Me | Me | 2 | CH₂CH₂OMe | SO₂Me |
| 6-70 | SO₂Me | Cl | 0 | CH₂CH₂OMe | SO₂Me |
| 6-71 | SO₂Me | Cl | 1 | CH₂CH₂OMe | SO₂Me |
| 6-72 | SO₂Me | Cl | 2 | CH₂CH₂OMe | SO₂Me |

TABLE 7
Compounds according to the invention of the general formula (I) in
the form of the sodium salts in which R² represents hydrogen, A
represents a direct bond, X¹ and X² each represent CH and X³
represents CR⁸

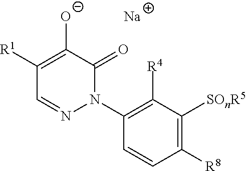

| No. | R¹ | R⁴ | n | R⁵ | R⁸ |
|---|---|---|---|---|---|
| 7-1 | Me | Me | 0 | CH₂—c-Pr | CF₃ |
| 7-2 | Me | Me | 1 | CH₂—c-Pr | CF₃ |
| 7-3 | Me | Me | 2 | CH₂—c-Pr | CF₃ |
| 7-4 | Me | Cl | 0 | CH₂—c-Pr | CF₃ |
| 7-5 | Me | Cl | 1 | CH₂—c-Pr | CF₃ |
| 7-6 | Me | Cl | 2 | CH₂—c-Pr | CF₃ |
| 7-7 | Me | Me | 0 | CH₂—c-Pr | SO₂Me |
| 7-8 | Me | Me | 1 | CH₂—c-Pr | SO₂Me |
| 7-9 | Me | Me | 2 | CH₂—c-Pr | SO₂Me |
| 7-10 | Me | Cl | 0 | CH₂—c-Pr | SO₂Me |
| 7-11 | Me | Cl | 1 | CH₂—c-Pr | SO₂Me |
| 7-12 | Me | Cl | 2 | CH₂—c-Pr | SO₂Me |
| 7-13 | SO₂Me | Me | 0 | CH₂—c-Pr | CF₃ |
| 7-14 | SO₂Me | Me | 1 | CH₂—c-Pr | CF₃ |
| 7-15 | SO₂Me | Me | 2 | CH₂—c-Pr | CF₃ |
| 7-16 | SO₂Me | Cl | 0 | CH₂—c-Pr | CF₃ |
| 7-17 | SO₂Me | Cl | 1 | CH₂—c-Pr | CF₃ |
| 7-18 | SO₂Me | Cl | 2 | CH₂—c-Pr | CF₃ |
| 7-19 | SO₂Me | Me | 0 | CH₂—c-Pr | SO₂Me |
| 7-20 | SO₂Me | Me | 1 | CH₂—c-Pr | SO₂Me |
| 7-21 | SO₂Me | Me | 2 | CH₂—c-Pr | SO₂Me |
| 7-22 | SO₂Me | Cl | 0 | CH₂—c-Pr | SO₂Me |
| 7-23 | SO₂Me | Cl | 1 | CH₂—c-Pr | SO₂Me |
| 7-24 | SO₂Me | Cl | 2 | CH₂—c-Pr | SO₂Me |
| 7-25 | Me | Me | 0 | CH₂CH₂OMe | CF₃ |
| 7-26 | Me | Me | 1 | CH₂CH₂OMe | CF₃ |
| 7-27 | Me | Me | 2 | CH₂CH₂OMe | CF₃ |
| 7-28 | Me | Cl | 0 | CH₂CH₂OMe | CF₃ |
| 7-29 | Me | Cl | 1 | CH₂CH₂OMe | CF₃ |
| 7-30 | Me | Cl | 2 | CH₂CH₂OMe | CF₃ |
| 7-31 | Me | Me | 0 | CH₂CH₂OMe | SO₂Me |
| 7-32 | Me | Me | 1 | CH₂CH₂OMe | SO₂Me |
| 7-33 | Me | Me | 2 | CH₂CH₂OMe | SO₂Me |
| 7-34 | Me | Cl | 0 | CH₂CH₂OMe | SO₂Me |
| 7-35 | Me | Cl | 1 | CH₂CH₂OMe | SO₂Me |
| 7-36 | Me | Cl | 2 | CH₂CH₂OMe | SO₂Me |
| 7-37 | SO₂Me | Me | 0 | CH₂CH₂OMe | CF₃ |
| 7-38 | SO₂Me | Me | 1 | CH₂CH₂OMe | CF₃ |
| 7-39 | SO₂Me | Me | 2 | CH₂CH₂OMe | CF₃ |
| 7-40 | SO₂Me | Cl | 0 | CH₂CH₂OMe | CF₃ |
| 7-41 | SO₂Me | Cl | 1 | CH₂CH₂OMe | CF₃ |
| 7-42 | SO₂Me | Cl | 2 | CH₂CH₂OMe | CF₃ |
| 7-43 | SO₂Me | Me | 0 | CH₂CH₂OMe | SO₂Me |
| 7-44 | SO₂Me | Me | 1 | CH₂CH₂OMe | SO₂Me |
| 7-45 | SO₂Me | Me | 2 | CH₂CH₂OMe | SO₂Me |
| 7-46 | SO₂Me | Cl | 0 | CH₂CH₂OMe | SO₂Me |
| 7-47 | SO₂Me | Cl | 1 | CH₂CH₂OMe | SO₂Me |
| 7-48 | SO₂Me | Cl | 2 | CH₂CH₂OMe | SO₂Me |

TABLE 8
Compounds according to the invention of the general formula (I) in
the form of the sodium salts in which R² represents hydrogen, A
represents —CH₂—, X¹ and X² each represent CH and X³
represents CR⁸

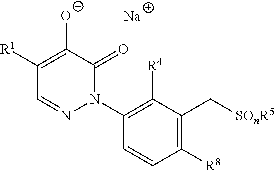

| No. | R¹ | R⁴ | n | R⁵ | R⁸ |
|---|---|---|---|---|---|
| 8-1 | Me | Me | 0 | Me | CF₃ |
| 8-2 | Me | Me | 1 | Me | CF₃ |
| 8-3 | Me | Me | 2 | Me | CF₃ |
| 8-4 | Me | Cl | 0 | Me | CF₃ |
| 8-5 | Me | Cl | 1 | Me | CF₃ |
| 8-6 | Me | Cl | 2 | Me | CF₃ |
| 8-7 | Me | Me | 0 | Me | SO₂Me |
| 8-8 | Me | Me | 1 | Me | SO₂Me |
| 8-9 | Me | Me | 2 | Me | SO₂Me |
| 8-10 | Me | Cl | 0 | Me | SO₂Me |
| 8-11 | Me | Cl | 1 | Me | SO₂Me |
| 8-12 | Me | Cl | 2 | Me | SO₂Me |
| 8-13 | SO₂Me | Me | 0 | Me | CF₃ |
| 8-14 | SO₂Me | Me | 1 | Me | CF₃ |
| 8-15 | SO₂Me | Me | 2 | Me | CF₃ |
| 8-16 | SO₂Me | Cl | 0 | Me | CF₃ |
| 8-17 | SO₂Me | Cl | 1 | Me | CF₃ |
| 8-18 | SO₂Me | Cl | 2 | Me | CF₃ |
| 8-19 | SO₂Me | Me | 0 | Me | SO₂Me |
| 8-20 | SO₂Me | Me | 1 | Me | SO₂Me |
| 8-21 | SO₂Me | Me | 2 | Me | SO₂Me |
| 8-22 | SO₂Me | Cl | 0 | Me | SO₂Me |
| 8-23 | SO₂Me | Cl | 1 | Me | SO₂Me |
| 8-24 | SO₂Me | Cl | 2 | Me | SO₂Me |
| 8-25 | Me | Me | 0 | CH₂CH₂OMe | CF₃ |
| 8-26 | Me | Me | 1 | CH₂CH₂OMe | CF₃ |
| 8-27 | Me | Me | 2 | CH₂CH₂OMe | CF₃ |
| 8-28 | Me | Cl | 0 | CH₂CH₂OMe | CF₃ |
| 8-29 | Me | Cl | 1 | CH₂CH₂OMe | CF₃ |
| 8-30 | Me | Cl | 2 | CH₂CH₂OMe | CF₃ |
| 8-31 | Me | Me | 0 | CH₂CH₂OMe | SO₂Me |
| 8-32 | Me | Me | 1 | CH₂CH₂OMe | SO₂Me |
| 8-33 | Me | Me | 2 | CH₂CH₂OMe | SO₂Me |
| 8-34 | Me | Cl | 0 | CH₂CH₂OMe | SO₂Me |
| 8-35 | Me | Cl | 1 | CH₂CH₂OMe | SO₂Me |
| 8-36 | Me | Cl | 2 | CH₂CH₂OMe | SO₂Me |
| 8-37 | SO₂Me | Me | 0 | CH₂CH₂OMe | CF₃ |
| 8-38 | SO₂Me | Me | 1 | CH₂CH₂OMe | CF₃ |
| 8-39 | SO₂Me | Me | 2 | CH₂CH₂OMe | CF₃ |
| 8-40 | SO₂Me | Cl | 0 | CH₂CH₂OMe | CF₃ |
| 8-41 | SO₂Me | Cl | 1 | CH₂CH₂OMe | CF₃ |
| 8-42 | SO₂Me | Cl | 2 | CH₂CH₂OMe | CF₃ |
| 8-43 | SO₂Me | Me | 0 | CH₂CH₂OMe | SO₂Me |
| 8-44 | SO₂Me | Me | 1 | CH₂CH₂OMe | SO₂Me |
| 8-45 | SO₂Me | Me | 2 | CH₂CH₂OMe | SO₂Me |
| 8-46 | SO₂Me | Cl | 0 | CH₂CH₂OMe | SO₂Me |
| 8-47 | SO₂Me | Cl | 1 | CH₂CH₂OMe | SO₂Me |
| 8-48 | SO₂Me | Cl | 2 | CH₂CH₂OMe | SO₂Me |

TABLE 9
Compounds according to the invention of the general formula (I) in
the form of the sodium salts in which R² represents hydrogen, A
represents —CH₂CH₂—, X¹ and X² each represent CH and X³
represents CR⁸

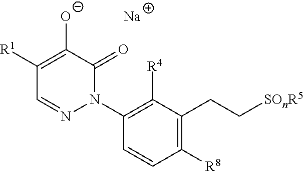

| No. | R¹ | R⁴ | n | R⁵ | R⁸ |
|---|---|---|---|---|---|
| 9-1 | Me | Me | 0 | Me | CF₃ |
| 9-2 | Me | Me | 1 | Me | CF₃ |
| 9-3 | Me | Me | 2 | Me | CF₃ |
| 9-4 | Me | Cl | 0 | Me | CF₃ |
| 9-5 | Me | Cl | 1 | Me | CF₃ |
| 9-6 | Me | Cl | 2 | Me | CF₃ |
| 9-7 | Me | Me | 0 | Me | SO₂Me |
| 9-8 | Me | Me | 1 | Me | SO₂Me |
| 9-9 | Me | Me | 2 | Me | SO₂Me |
| 9-10 | Me | Cl | 0 | Me | SO₂Me |
| 9-11 | Me | Cl | 1 | Me | SO₂Me |
| 9-12 | Me | Cl | 2 | Me | SO₂Me |
| 9-13 | SO₂Me | Me | 0 | Me | CF₃ |
| 9-14 | SO₂Me | Me | 1 | Me | CF₃ |
| 9-15 | SO₂Me | Me | 2 | Me | CF₃ |
| 9-16 | SO₂Me | Cl | 0 | Me | CF₃ |
| 9-17 | SO₂Me | Cl | 1 | Me | CF₃ |
| 9-18 | SO₂Me | Cl | 2 | Me | CF₃ |
| 9-19 | SO₂Me | Me | 0 | Me | SO₂Me |
| 9-20 | SO₂Me | Me | 1 | Me | SO₂Me |
| 9-21 | SO₂Me | Me | 2 | Me | SO₂Me |
| 9-22 | SO₂Me | Cl | 0 | Me | SO₂Me |
| 9-23 | SO₂Me | Cl | 1 | Me | SO₂Me |
| 9-24 | SO₂Me | Cl | 2 | Me | SO₂Me |
| 9-25 | Me | Me | 0 | CH₂CH₂OMe | CF₃ |
| 9-26 | Me | Me | 1 | CH₂CH₂OMe | CF₃ |
| 9-27 | Me | Me | 2 | CH₂CH₂OMe | CF₃ |
| 9-28 | Me | Cl | 0 | CH₂CH₂OMe | CF₃ |
| 9-29 | Me | Cl | 1 | CH₂CH₂OMe | CF₃ |
| 9-30 | Me | Cl | 2 | CH₂CH₂OMe | CF₃ |
| 9-31 | Me | Me | 0 | CH₂CH₂OMe | SO₂Me |
| 9-32 | Me | Me | 1 | CH₂CH₂OMe | SO₂Me |
| 9-33 | Me | Me | 2 | CH₂CH₂OMe | SO₂Me |
| 9-34 | Me | Cl | 0 | CH₂CH₂OMe | SO₂Me |
| 9-35 | Me | Cl | 1 | CH₂CH₂OMe | SO₂Me |
| 9-36 | Me | Cl | 2 | CH₂CH₂OMe | SO₂Me |
| 9-37 | SO₂Me | Me | 0 | CH₂CH₂OMe | CF₃ |
| 9-38 | SO₂Me | Me | 1 | CH₂CH₂OMe | CF₃ |
| 9-39 | SO₂Me | Me | 2 | CH₂CH₂OMe | CF₃ |
| 9-40 | SO₂Me | Cl | 0 | CH₂CH₂OMe | CF₃ |
| 9-41 | SO₂Me | Cl | 1 | CH₂CH₂OMe | CF₃ |
| 9-42 | SO₂Me | Cl | 2 | CH₂CH₂OMe | CF₃ |
| 9-43 | SO₂Me | Me | 0 | CH₂CH₂OMe | SO₂Me |
| 9-44 | SO₂Me | Me | 1 | CH₂CH₂OMe | SO₂Me |
| 9-45 | SO₂Me | Me | 2 | CH₂CH₂OMe | SO₂Me |
| 9-46 | SO₂Me | Cl | 0 | CH₂CH₂OMe | SO₂Me |
| 9-47 | SO₂Me | Cl | 1 | CH₂CH₂OMe | SO₂Me |
| 9-48 | SO₂Me | Cl | 2 | CH₂CH₂OMe | SO₂Me |

### A. CHEMICAL EXAMPLES

### Preparation of 5-chloro-2-{3-[(cyclopropylmethyl)sulfanyl]-2-methyl-4-(trifluoromethyl)phenyl}-4-hydroxypyridazin-3(2H)-one (Example No. 1-499)

### Step 1: Synthesis of 6-bromo-2-fluoro-3-(trifluoromethyl)benzaldehyde

At −78° C., 181.07 ml of a 2.5M (452.7 mmol) solution of n-butyllithium were added dropwise to a solution of 63.9 g (452.7 mmol) of 2,2,6,6-tetramethylpiperidine in 833 ml of dry THF. The mixture was stirred at this temperature for 30 min. 100.0 g (411.5 mmol) of 4-bromo-2-fluoro-1-(trifluoromethyl)benzene were then added dropwise at −78° C. The mixture was stirred at this temperature for 2 h. 33.1 g (452.7 mmol) of DMF were then added dropwise at −78° C. The reaction mixture was then stirred for 2 h. For work-up, 300 ml of water were added to the contents. The mixture was extracted three times with in each case 200 ml of dichloromethane. The combined organic phases were washed with 300 ml of 1M hydrochloric acid and then with 300 ml of a saturated aqueous sodium chloride solution. The organic phase was dried and the filtrate was freed of the solvent. 96.2 g of the desired product were obtained.

### Step 2: Synthesis of 6-bromo-2-(tert-butylsulfanyl)-3-(trifluoromethyl)benzaldehyde

At 0° C., 30.3 g (335.8 mmol) of tert-butylmercaptan were added to a solution of 65.0 g (239.8 mmol) of 6-bromo-2-fluoro-3-(trifluoromethyl)benzaldehyde and 66.3 g (479.7 mmol) of potassium carbonate in 500 ml of N,N-dimethylformamide. The mixture was stirred at this temperature for 12 h. Subsequently, 15.6 g (48.0 mmol) of cesium carbonate were added and the mixture was stirred for a further 3 h. For work-up, 1 I of water was added to the contents. The mixture was extracted three times with in each case 300 ml of dichloromethane. The combined organic phases were washed four times with in each case 300 ml of a saturated aqueous sodium chloride solution. The organic phase was dried and the filtrate was freed of the solvent, giving 68 g of the desired product.

### Step 3: Synthesis of [6-bromo-2-(tert-butylsulfanyl)-3-(trifluoromethyl)phenyl]methanol

At −10° C., 3.49 g (92.3 mmol) of sodium borohydride were added slowly to a solution of 63.0 g (184.7 mmol) of 6-bromo-2-(tert-butylsulfanyl)-3-(trifluoromethyl)benzaldehyde in 500 ml of methanol. After the reaction had been checked showing complete conversion, 3M hydrochloric acid was added to work-up the contents. The mixture was concentrated and the residue was poured onto 400 ml of water. The mixture was extracted twice with in each case 300 ml of dichloromethane. The combined organic phases were washed with a saturated aqueous sodium chloride solution and dried, and the filtrate was then freed of the solvent. 60.0 g of the desired product were obtained.

### Step 4: Synthesis of 6-bromo-2-(tert-butylsulfanyl)-3-(trifluoromethyl)benzyl methanesulfonate

At 0° C., 31.3 g (272.8 mmol) of methanesulfonyl chloride were added dropwise to a solution of 60.0 g (174.8 mmol) of [6-bromo-2-(tert-butylsulfanyl)-3-(trifluoromethyl)phenyl]methanol and 44.2 g (437.1 mmol) of triethylamine in 500 ml of dichloromethane. After the reaction had been checked showing complete conversion, the solution was, for work-up, washed twice with in each case 300 ml of water and dried, and the filtrate was freed of the solvent. 70.0 g of the desired product were obtained.

### Step 5: Synthesis of 1-bromo-3-(tert-butylsulfanyl)-2-methyl-4-(trifluoromethyl)benzene

At −10° C., a solution of 70.0 g (166.2 mmol) of 6-bromo-2-(tert-butylsulfanyl)-3-(trifluoromethyl)benzyl methanesulfonate in 100 ml of dry THF was added dropwise to a solution of 6.94 g (182.8 mmol) of lithium aluminum hydride in 500 ml of dry THF. The content was stirred for 1 h. For work-up, sodium sulfate decahydrate was added until no more evolution of gas could be observed. The mixture was filtered and the filtrate was dried. The filtrate was then freed of the solvent and the residue was purified chromatographically, giving 45.0 g of the desired product.

### Step 6: Synthesis of 3-bromo-2-methyl-6-(trifluoromethyl)benzenethiol

23.7 g (137.5 mmol) of 4-methylbenzenesulfonic acid were added to a solution of 45.0 g (137.5 mmol) of 1-bromo-3-(tert-butylsulfanyl)-2-methyl-4-(trifluoromethyl)benzene in 175 ml of toluene. The mixture was heated under reflux for 2 h. The solvent was removed on a rotary evaporator and the residue was dissolved in 200 ml of dichloromethane. The solution was extracted four times with 15% strength aqueous potassium hydroxide solution. The combined aqueous phases were acidified with concentrated hydrochloric acid and the product was then extracted with dichloromethane. The organic phase was dried and filtered and the filtrate was freed of the solvent, giving 32.0 g of the desired product.

### Step 7: Synthesis of 1-bromo-3-[(cyclopropylmethyl)sulfanyl]-2-methyl-4-(trifluoromethyl)benzene

14.0 g (103.6 mmol) of (bromomethyl)cyclopropane were added to a mixture of 20 g (74.1 mmol) of 3-bromo-2-methyl-6-(trifluoromethyl)benzenethiol and 36 g (111.1 mmol) of cesium carbonate in 80 ml of acetonitrile. The content was stirred at 80° C. for 2 h. For work-up, the mixture was filtered and the filtrate was freed from the solvent. The residue was purified chromatographically, which gave 20.0 g of the desired product.

### Step 8: Synthesis of 1-{3-[(cyclopropylmethyl)sulfanyl]-2-methyl-4-(trifluoromethyl)phenyl}-2-(diphenylmethylene)hydrazine

A mixture of 20 mg (0.2 mmol) of sodium tert-butoxide and 33 mg (0.17 mmol) of benzophenone hydrazine was added to a solution of 50 mg (0.15 mmol) of 1-bromo-3-[(cyclopropylmethyl)sulfanyl]-2-methyl-4-(trifluoromethyl)benzene in 1 ml of toluene. To remove oxygen, the mixture was then degassed for 10 min. Subsequently, 1 mg (0.002 mmol) of 2,2′-bis(diphenylphosphino)-1,1′-binaphthyl was added under protective gas. To remove oxygen, the mixture was degassed for 15 min. Subsequently, 0.22 mg (0.001 mmol) of palladium(II) acetate was added under protective gas. Under protective gas, the content was heated to a temperature of 90° C. for 3 h. Work-up and purification gave 35 mg of the desired product.

### Step 9: Synthesis of {3-[(cyclopropylmethyl)sulfanyl]-2-methyl-4-(trifluoromethyl)-phenyl}hydrazine

A solution of 30 mg (0.11 mmol) of 1-{3-[(cyclopropylmethyl)sulfanyl]-2-methyl-4-(trifluoromethyl)phenyl}-2-(diphenylmethylene)hydrazine in 2 ml of isopropyl alcohol and 2 ml of concentrated hydrochloric acid was stirred at room temperature for 48 h. Work-up and purification gave 10 mg of the desired product.

### Step 10: Synthesis of 4,5-dichloro-2-{3-[(cyclopropylmethyl)sulfanyl]-2-methyl-4-(trifluoromethyl)phenyl}pyridazin-3(2H)-one

34 mg (0.2 mmol, 1.1 eq) of 3,4-dichloro-5-hydroxyfuran-2(5H)-one were added to a solution of 50 mg (0.18 mmol) of {3-[(cyclopropylmethyl)sulfanyl]-2-methyl-4-(trifluoromethyl)phenyl}hydrazine in 1 ml of ethanol. The mixture was stirred at room temperature for 3 h. 1 ml of acetic acid was then added, and the mixture was heated under reflux for 3 h. Work-up and purification gave 40 mg of the desired product.

### Step 11: Synthesis of 5-chloro-2-{3-[(cyclopropylmethyl)sulfanyl]-2-methyl-4-(trifluoromethyl)phenyl}-4-methoxypyridazin-3(2H)-one

0.033 ml (20%, 0.12 mmol) of a solution of sodium methoxide in methanol was added to a solution of 50 mg (0.12 mmol) of 4,5-dichloro-2-{3-[(cyclopropylmethyl)sulfanyl]-2-methyl-4-(trifluoromethyl)phenyl}pyridazin-3(2H)-one in 2 ml of dry dioxane. At a temperature of 15° C., the mixture was diluted with 5 ml of dry dioxane. The reaction mixture was then stirred at a temperature of 15° C. for another 1 h. Work-up and purification gave 23 mg of the desired product.

### Step 12: Synthesis of 5-chloro-2-{3-[(cyclopropylmethyl)sulfanyl]-2-methyl-4-(trifluoromethyl)phenyl}-4-hydroxypyridazin-3(2H)-one (Example No. 1-499)

At a temperature of 0° C., 18.6 mg (0.074 mmol) of boron tribromide, as a 1M solution in dichloromethane, were added to a solution of 10 mg (0.02 mmol) of 5-chloro-2-{3-[(cyclopropymethyl)sulfanyl]-2-methyl-4-(trifluoromethyl)phenyl}-4-methoxypyridazin-3(2H)-one in 1 ml of dichloromethane. The mixture was stirred at room temperature for 1 h. Work-up and purification gave 4 mg of the desired product.

NMR data of selected examples

NMR peak list method

The 1H NMR data of selected examples are stated in the form of 1H NMR peak lists. For each signal peak, first the 6 value in ppm and then the signal intensity in round brackets are listed. The pairs of 5 value-signal intensity numbers for different signal peaks are listed with separation from one another by semicolons.

The peak list for one example therefore has the form of:

δ₁ (intensity₁); δ₂ (intensity₂); . . . ; δᵢ (intensityᵢ); . . . ; δₙ (intensityₙ)

The intensity of sharp signals correlates with the height of the signals in a printed example of an NMR spectrum in cm and shows the true ratios of the signal intensities. In the case of broad signals, several peaks or the middle of the signal and the relative intensity thereof may be shown in comparison to the most intense signal in the spectrum.

To calibrate the chemical shift of 1H NMR spectra, we used tetramethylsilane and/or the chemical shift of the solvent, in particular in the case of spectra measured in DMSO. Accordingly, the tetramethylsilane peak may be present in NMR peak lists, but it does not have to be.

The lists of the 1H NMR peaks are similar to the conventional ¹H-NMR printouts and thus usually contain all peaks listed in a conventional NMR interpretation.

In addition, like conventional 1H NMR printouts, they may show solvent signals, signals of stereoisomers of the target compounds which likewise form part of the subject matter of the invention, and/or peaks of impurities.

When stating compound signals in the delta range of solvents and/or water, in our lists of 1H NMR peaks, the usual solvent peaks, for example peaks of DMSO in DMSO-D₆ and the peak of water are shown, which usually have on average a high intensity.

The peaks of stereoisomers of the target compounds and/or peaks of impurities usually have a lower intensity on average than the peaks of the target compounds (for example with a purity of >90%).

Such stereoisomers and/or impurities may be typical of the particular preparation process. Their peaks can thus help in identifying reproduction of our preparation process with reference to “by-product fingerprints”.

An expert calculating the peaks of the target compounds by known methods (MestreC, ACD simulation, but also with empirically evaluated expected values) can, if required, isolate the peaks of the target compounds, optionally using additional intensity filters. This isolation would be similar to the peak picking in question in conventional 1H NMR interpretation.

Further details on 1H NMR peak lists are available from Research Disclosure Database Number 564025.

Example 1-499: ¹H NMP(400.0 MHz, CDCl₃):
δ = 7.910(11.2); 7.677(4.9); 7.656(4.8); 7.518(7.4); 7.380(1.2);
7.296(6.0); 7.290(4.4); 7.276(7.9); 7.259(1356.1); 7.226(2.8); 7.209(3.3);
7.140(1.7); 6.995(7.4); 3.731(3.6); 3.487(1.3); 2.629(2.7); 2.540(4.9);
2.314(2.0); 2.160(14.3); 1.679(2.9); 1.284(2.8); 1.254(16.0); 0.978(1.6);
0.877(3.9); 0.861(3.4); 0.503(2.6); 0.345(3.7); 0.146(3.3); 0.120(2.6);
0.008(16.4); 0.000(542.8); −0.009(21.4); −0.033(4.7); −0.150(2.3)
Example 2-139: ¹H NMP(400.0 MHz, CDCl₃):
δ = 7.910(2.8); 7.783(2.2); 7.578(3.7); 7.518(5.6); 7.259(785.6); 6.995
(4.1); 3.878(4.5); 3.731(2.6); 2.887(1.5); 2.115(16.0); 2.003(6.0); 1.852
(4.2); 1.254(3.3); 0.146(1.4); 0.008(12.3); 0.000(389.1); −0.009
(13.9); −0.149(1.4)

### B. FORMULATION EXAMPLES

a) A dusting product is obtained by mixing 10 parts by weight of a compound of the formula (I) and/or salts thereof and 90 parts by weight of talc as an inert substance and comminuting the mixture in a hammer mill.
b) A readily water-dispersible, wettable powder is obtained by mixing 25 parts by weight of a compound of the formula (I) and/or salts thereof, 64 parts by weight of kaolin-containing quartz as an inert substance, 10 parts by weight of potassium lignosulfonate and 1 part by weight of sodium oleoylmethyltaurate as a wetting agent and dispersant, and grinding the mixture in a pinned-disk mill.
c) A readily water-dispersible dispersion concentrate is obtained by mixing parts by weight of a compound of the formula (I) and/or salts thereof with 6 parts by weight of alkylphenol polyglycol ether (®Triton X 207), 3 parts by weight of isotridecanol polyglycol ether (8 EO) and 71 parts by weight of paraffinic mineral oil (boiling range for example about 255 to above 277 C), and grinding the mixture in a ball mill to a fineness of below 5 microns.
d) An emulsifiable concentrate is obtained from 15 parts by weight of a compound of the formula (I) and/or salts thereof, 75 parts by weight of cyclohexanone as a solvent and 10 parts by weight of ethoxylated nonylphenol as an emulsifier.
e) Water-dispersible granules are obtained by mixing
   75 parts by weight of a compound of the formula (I) and/or salts thereof,
   parts by weight of calcium lignosulfonate,
   parts by weight of sodium lauryl sulfate,
   3 parts by weight of polyvinyl alcohol and
   7 parts by weight of kaolin,
   grinding the mixture in a pinned-disk mill, and granulating the powder in a fluidized bed by spray application of water as a granulating liquid.
f) Water-dispersible granules are also obtained by homogenizing and precomminuting, in a colloid mill,
   parts by weight of a compound of the formula (I) and/or salts thereof,
   parts by weight of sodium 2,2′-dinaphthylmethane-6,6′-disulfonate
   2 parts by weight of sodium oleoylmethyltaurate,
   1 part by weight of polyvinyl alcohol
   17 parts by weight of calcium carbonate and
   50 parts by weight of water,
   then grinding the mixture in a bead mill and atomizing and drying the resulting suspension in a spray tower by means of a one-phase nozzle.

### C. BIOLOGICAL EXAMPLES

### 1. Pre-Emergence Herbicidal Action Against Harmful Plants

Seeds of monocotyledonous and dicotyledonous weed plants and crop plants are laid out in wood-fiber pots in sandy loam and covered with soil. The compounds of the invention, formulated in the form of wettable powders (WP) or as emulsion concentrates (EC), are then applied to the surface of the covering soil in the form of an aqueous suspension or emulsion at a water application rate equating to 600 to 800 I/ha, with addition of 0.2% wetting agent. After the treatment, the pots are placed in a greenhouse and kept under good growth conditions for the trial plants. The damage to the test plants is scored visually after a test period of 3 weeks by comparison with untreated controls (herbicidal activity in percent (%): 100% activity=the plants have died, 0% activity=like control plants). Here, for example, the compounds Nos. 1-499 and 2-139 showed, at an application rate of 0.32 kg of active substance or less per hectare, very good activity (80% to 100% of herbicidal activity) against harmful plants such as Amaranthus retroflexus, Echinochloa crus-galli, Setaria viridis and Abutilon theophrasti. At the same time, the compounds according to the invention leave gramineous crops such as barley, wheat, rye, millet, corn or rice virtually undamaged even at high active compound dosages when applied by the pre-emergence method. In addition, some substances also spare dicotyledonous crops such as soybeans, cotton, oilseed rape, sugar beet or potatoes.

Some of the compounds according to the invention have high selectivity and are therefore suitable for controlling unwanted vegetation in agricultural crops by the pre-emergence method.

### 2. Post-Emergence Herbicidal Action Against Harmful Plants

Seeds of monocotyledonous and dicotyledonous weed and crop plants are laid out in sandy loam in wood-fiber pots, covered with soil and cultivated in a greenhouse under good growth conditions. 2 to 3 weeks after sowing, the test plants are treated at the one-leaf stage. The compounds of the invention, formulated in the form of wettable powders (WP) or as emulsion concentrates (EC), are then sprayed onto the green parts of the plants in the form of an aqueous suspension or emulsion at a water application rate equating to 600 to 800 I/ha, with addition of 0.2% wetting agent. After the test plants have been left to stand in the greenhouse under optimal growth conditions for about 3 weeks, the action of the preparations is assessed visually in comparison to untreated controls (herbicidal action in percent (%): 100% activity=the plants have died, 0% activity=like control plants). Here, for example, the compounds Nos. 1-499 and 2-139 showed, at an application rate of 0.08 kg of active substance or less per hectare, very good herbicidal activity (80% to 100% herbicidal activity) against harmful plants such as Pharbitis purpureum, Echinochloa crus-galli, Setaria viridis, Amaranthus retroflexus, Abutilon theophrasti, Viola tricolor, Veronica persica and Stellaria media. At the same time, the compounds according to the invention leave gramineous crops such as barley, wheat, rye, millet, corn or rice virtually undamaged even at high active compound dosages when applied by the post-emergence method. In addition, some substances also spare dicotyledonous crops such as soybeans, cotton, oilseed rape, sugar beets or potatoes.

Some of the compounds according to the invention have high selectivity and are therefore suitable for controlling unwanted vegetation in agricultural crops by the post-emergence method.

1. A 2-(hetero)arylpyridazinone of formula (I) or a salt thereof 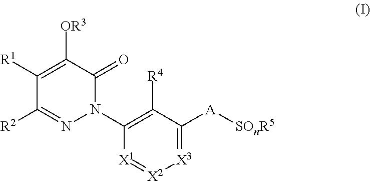
in which
R¹ represents hydrogen, halogen, cyano (C₁-C₆)-alkyl, (C₃-C₆)-cycloalkyl, (C₂-C₆)-alkenyl, (C₄-C₆)-cycloalkenyl, (C₂-C₆)-alkynyl, halo-(C₁-C₆)-alkyl, (C₁-C₆)-alkoxy, (C₁-C₆)-alkoxy-(C₁-C₃)-alkyl, (C₁-C₆)-alkoxy-(C₂-C₆)-alkoxy, (C₁-C₆)-alkoxy-(C₂-C₆)-alkoxy-(C₁-C₃)-alkyl, (C₃-C₆)-cycloalkyl-(C₁-C₃)-alkyl, amino, (C₁-C₆)-alkylamino, di-(C₁-C₆)-alkylamino, (C₁-C₃)-alkyl-(O)C-amino-(C₁-C₄)-alkyl, (C₁-C₆)-alkyl-(O)ₙS, (C₁-C₆)-alkyl-(O)ₙS—(C₁-C₃)-alkyl, halo-(C₁-C₆)-alkyl-(O)ₙS or halo-(C₁-C₆)-alkyl-(O)ₙS—(C₁-C₃)-alkyl;
R² represents hydrogen, hydroxy, halogen, nitro, amino, cyano, (C₁-C₆)-alkyl, (C₁-C₃)-alkoxy, (C₃-C₆)-cycloalkyl, (C₂-C₆)-alkenyl, (C₂-C₆)-alkynyl, halo-(C₁-C₆)-alkyl, (C₁-C₆)-alkoxy-(C₁-C₃)-alkyl, (C₃-C₆)-cycloalkyl-(C₁-C₃)-alkyl, (C₁-C₆)-alkyl-(O)ₙS, (C₁-C₆)-alkyl-(O)ₙS—(C₁-C₃)-alkyl, halo-(C₁-C₆)-alkyl-(O)ₙS, halo-(C₁-C₆)-alkyl-(O)ₙS—(C₁-C₃)-alkyl, (C₁-C₃)-alkylamino or di-(C₁-C₃)-alkylamino;
R³ represents hydrogen, (C₁-C₆)-alkyl-(O)C, aryl-(O)C, (C₁-C₆)-alkoxy-(O)C, (C₁-C₆)-alkyl-(O)ₙS, (C₁-C₆)-alkyl-(O)ₙS(O)C or aryl-(O)ₙS, where the aryl groups are in each case substituted by s radicals R⁹;
R⁴ represents hydroxy, halogen, cyano, nitro, (C₁-C₆)-alkyl, (C₃-C₆)-cycloalkyl, halo-(C₁-C₆)-alkyl, (C₂-C₆)-alkenyl, halo-(C₂-C₆)-alkenyl, (C₂-C₆)-alkynyl, (C₁-C₆)-alkoxy, (C₂-C₆)-alkenyloxy, (C₃-C₆)-cycloalkyl-(C₁-C₃)-alkyl, (C₁-C₆)-alkoxy-(C₁-C₃)-alkyl, (C₁-C₆)-alkoxy-(C₂-C₆)-alkoxy, (C₁-C₆)-alkoxy-(C₂-C₆)-alkoxy-(C₁-C₃)-alkyl, halo-(C₁-C₆)-alkoxy, halo-(C₁-C₆)-alkoxy-(C₁-C₃)-alkyl, (C₁-C₆)-alkyl-(O)ₙS, halo-(C₁-C₆)-alkyl-(O)ₙS, aryl, aryl-(O)ₙS, heterocyclyl, heterocyclyl-(O)ₙS, aryloxy, aryl-(C₂-C₆)-alkyl, aryl-(C₁-C₆)-alkoxy, heterocyclyloxy, heterocyclyl-(C₁-C₃)-alkoxy-(C₁-C₃)-alkyl, HO(O)C, HO(O)C—(C₁-C₃)-alkoxy, (C₁-C₃)-alkoxy-(O)C, (C₁-C₃)-alkoxy-(O)C—(C₁-C₃)-alkoxy, (C₁-C₃)-alkylamino, di-(C₁-C₃)-alkylamino, (C₁-C₃)-alkylamino-(O)ₙS, (C₁-C₃)-alkylamino-(O)ₙS—(C₁-C₃)-alkyl, di-(C₁-C₃)-alkylamino-(O)ₙS, di-(C₁-C₃)-alkylamino-(O)ₙS—(C₁-C₃)-alkyl, (C₁-C₃)-alkylamino-(O)C, (C₁-C₃)-alkylamino-(O)C—(C₁-C₃)-alkyl, di-(C₁-C₃)-alkylamino-(O)C, di-(C₁-C₃)-alkylamino-(O)C—(C₁-C₃)-alkyl, (C₁-C₃)-alkyl-(O)C-amino, (C₁-C₃)-alkyl-(O)ₙS-amino, (C₁-C₃)-alkyl-(O)ₙS—(C₁-C₃)-alkylamino or (C₁-C₃)-alkyl-(O)ₙS-amino-(C₁-C₃)-alkyl, where the heterocyclyl groups and aryl groups are substituted by s radicals from the group consisting of (C₁-C₃)-alkyl, halo-(C₁-C₃)-alkyl, (C₁-C₃)-alkoxy, halo-(C₁-C₃)-alkoxy, phenyl, cyano, nitro and halogen;
A represents a direct bond or (C₁-C₄)-alkylene, where the methylene groups in (C₁-C₄)-alkylene independently of one another may carry n radicals from the group consisting of halogen, (C₁-C₄)-alkyl, halo-(C₁-C₄)-alkyl, (C₁-C₄)-alkoxy, halo-(C₁-C₄)-alkoxy or (C₁-C₄)-alkoxy-(C₁-C₄)-alkyl;
R⁵ represents (C₁-C₆)-alkyl, (C₃-C₆)-cycloalkyl, (C₃-C₆)-cycloalkyl-(C₁-C₆)-alkyl, (C₁-C₆)-alkoxy-(C₁-C₆)-alkyl;
X¹ represents N or CR⁶;
X² represents N or CR⁷;
X³ represents N or CR⁸;
R⁶ represents hydrogen, halogen, (C₁-C₃)-alkyl, (C₁-C₃)-alkoxy, (C₂-C₃)-alkenyl, (C₂-C₃)-alkynyl, halo-(C₁-C₃)-alkyl, halo-(C₁-C₃)-alkoxy;
R⁷ represents hydrogen, halogen, (C₁-C₃)-alkyl;
R⁸ represents hydrogen, hydroxy, halogen, cyano, nitro, (C₁-C₆)-alkyl, (C₃-C₆)-cycloalkyl, halo-(C₁-C₆)-alkyl, (C₂-C₆)-alkenyl, halo-(C₂-C₆)-alkenyl, (C₂-C₆)-alkynyl, (C₁-C₆)-alkoxy, (C₂-C₆)-alkenyloxy, (C₃-C₆)-cycloalkyl-(C₁-C₃)-alkyl, (C₁-C₆)-alkoxy-(C₁-C₃)-alkyl, (C₁-C₆)-alkoxy-(C₂-C₆)-alkoxy, (C₁-C₆)-alkoxy-(C₂-C₆)-alkoxy-(C₁-C₃)-alkyl, halo-(C₁-C₆)-alkoxy, halo-(C₁-C₆)-alkoxy-(C₁-C₃)-alkyl, (C₁-C₆)-alkyl-(O)ₙS, halo-(C₁-C₆)-alkyl-(O)ₙS, aryl, aryl-(O)ₙS, heterocyclyl, heterocyclyl-(O)ₙS, aryloxy, aryl-(C₂-C₆)-alkyl, aryl-(C₁-C₆)-alkoxy, heterocyclyloxy, heterocyclyl-(C₁-C₃)-alkoxy-(C₁-C₃)-alkyl, HO(O)C, HO(O)C—(C₁-C₃)-alkoxy, (C₁-C₃)-alkoxy-(O)C, (C₁-C₃)-alkoxy-(O)C—(C₁-C₃)-alkoxy, (C₁-C₃)-alkylamino, di-(C₁-C₃)-alkylamino, (C₁-C₃)-alkylamino-(O)ₙS, (C₁-C₃)-alkylamino-(O)ₙS—(C₁-C₃)-alkyl, di-(C₁—C)-alkylamino-(O)ₙS, di-(C₁-C₃)-alkylamino-(O)ₙS—(C₁-C₃)-alkyl, (C₁-C₃)-alkylamino-(O)C, (C₁-C₃)-alkylamino-(O)C—(C₁-C₃)-alkyl, di-(C₁-C₃)-alkylamino-(O)C, di-(C₁-C₃)-alkylamino-(O)C—(C₁-C₃)-alkyl, (C₁-C₃)-alkyl-(O)C-amino, (C₁-C₃)-alkyl-(O)ₙS-amino, (C₁-C₃)-alkyl-(O)ₙS—(C₁-C₃)-alkylamino or (C₁-C₃)-alkyl-(O)ₙS-amino-(C₁-C₃)-alkyl, where the heterocyclyl groups and aryl groups are substituted by s radicals from the group consisting of (C₁-C₃)-alkyl, halo-(C₁-C₃)-alkyl, (C₁-C₃)-alkoxy, halo-(C₁-C₃)-alkoxy, (C₁-C₆)-alkyl-(O)ₙS, phenyl, cyano, nitro and halogen,
or
R⁷ and R⁸ together with the carbon atoms to which they are attached represent an unsaturated five- or six-membered ring which contains s nitrogen atoms and is substituted by s radicals R¹⁰;
R⁹ represents halogen, (C₁-C₃)-alkyl, halo-(C₁-C₃)-alkyl, (C₁-C₆)-alkoxy,
R¹⁰ represents cyano, halogen, (C₁-C₃)-alkyl-(O)ₙS, (C₁-C₃)-alkyl, (C₂-C₃)-alkenyl, (C₂-C₃)-alkynyl, halo-(C₁-C₃)-alkyl or morpholinyl;
n represents 0, 1 or 2;
s represents 0, 1, 2 or 3,
with the proviso that R⁵ does not represent (C₁-C₆)-alkyl if A represents a direct bond.

2. The 2-(hetero)arylpyridazinone or salt as claimed in claim 1 in which
R¹ represents hydrogen, halogen, cyano, (C₁-C₆)-alkyl, (C₃-C₆)-cycloalkyl, (C₂-C₆)-alkenyl, (C₂-C₆)-alkynyl, halo-(C₁-C₆)-alkyl, (C₁-C₆)-alkoxy-(C₁-C₃)-alkyl, (C₃-C₆)-cycloalkyl-(C₁-C₃)-alkyl, amino or (C₁-C₆)-alkyl-(O)ₙS;
R² represents hydrogen, halogen, cyano, (C₁-C₆)-alkyl, (C₂-C₆)-alkenyl, (C₂-C₆)-alkynyl, halo-(C₁-C₆)-alkyl or (C₁-C₆)-alkyl-(O)ₙS;
R³ represents hydrogen,
R⁴ represents hydroxy, halogen, cyano, nitro, (C₁-C₆)-alkyl, (C₃-C₆)-cycloalkyl, halo-(C₁-C₆)-alkyl, (C₂-C₆)-alkenyl, (C₂-C₆)-alkynyl, (C₁-C₆)-alkoxy, (C₁-C₆)-alkoxy-(C₁-C₃)-alkyl, (C₁-C₆)-alkoxy-(C₂-C₆)-alkoxy, (C₁-C₆)-alkoxy-(C₂-C₆)-alkoxy-(C₁-C₃)-alkyl, halo-(C₁-C₆)-alkoxy, halo-(C₁-C₆)-alkoxy-(C₁-C₃)-alkyl, (C₁-C₆)-alkyl-(O)S, halo-(C₁-C₆)-alkyl-(O)ₙS, aryl, heterocyclyl, aryloxy, heterocyclyl-(C₁-C₃)-alkoxy-(C₁-C₃)-alkyl, (C₁-C₃)-alkylamino, di-(C₁-C₃)-alkylamino, (C₁-C₃)-alkylamino-(O)ₙS, (C₁-C₃)-alkylamino-(O)ₙS—(C₁-C₃)-alkyl, di-(C₁-C₃)-alkylamino-(O)ₙS, di-(C₁-C₃)-alkylamino-(O)ₙS—(C₁-C₃)-alkyl, (C₁-C₃)-alkylamino-(O)C, di-(C₁-C₃)-alkylamino-(O)C, di-(C₁-C₃)-alkylamino-(O)C—(C₁-C₃)-alkyl, (C₁-C₃)-alkyl-(O)C-amino or (C₁-C₃)-alkyl-(O)ₙS-amino, where the heterocyclyl groups and aryl groups are substituted by s radicals from the group consisting of (C₁-C₃)-alkyl, halo-(C₁-C₃)-alkyl, (C₁-C₃)-alkoxy, halo-(C₁-C₃)-alkoxy, cyano, nitro and halogen;
A represents a direct bond or (C₁-C₄)-alkylene;
R⁵ represents (C₁-C₆)-alkyl, (C₃-C₆)-cycloalkyl, (C₃-C₆)-cycloalkyl-(C₁-C₆)-alkyl, (C₁-C₆)-alkoxy-(C₁-C₆)-alkyl;
X¹ represents CR⁶;
X² represents CR⁷;
X³ represents CR⁸;
R⁶ and R⁷ independently of one another represent hydrogen, halogen, or (C₁-C₃)-alkyl;
R⁸ represents hydrogen, halogen, nitro, (C₁-C₆)-alkyl, (C₃-C₆)-cycloalkyl, halo-(C₁-C₆)-alkyl, (C₂-C₆)-alkenyl, halo-(C₂-C₆)-alkenyl, (C₂-C₆)-alkynyl, (C₁-C₆)-alkoxy, (C₂-C₆)-alkenyloxy, (C₃-C₆)-cycloalkyl-(C₁-C₃)-alkyl, (C₁-C₆)-alkoxy-(C₁-C₃)-alkyl, (C₁-C₆)-alkoxy-(C₂-C₆)-alkoxy, halo-(C₁-C₆)-alkoxy, (C₁-C₆)-alkyl-(O)ₙS or phenyl, where the phenyl group is substituted by s radicals from the group consisting of (C₁-C₃)-alkyl, halo-(C₁-C₃)-alkyl, (C₁-C₃)-alkoxy, halo-(C₁-C₃)-alkoxy, (C₁-C₆)-alkyl-(O)ₙS, phenyl, cyano, nitro and halogen;
n represents 0, 1 or 2;
s represents 0, 1, 2 or 3.

3. The 2-(hetero)arylpyridazinone or salt as claimed in claim 1 in which
R¹ represents hydrogen, amino, chlorine, bromine, cyano, methyl, ethyl, isopropyl, cyclopropyl, vinyl, propargyl, isopropenyl or methyl-(O)ₙS;
R² represents hydrogen, halogen or (C₁-C₆)-alkyl,
R³ represents hydrogen,
R⁴ represents fluorine, chlorine, cyano, nitro, methyl, trifluoromethyl, 2-fluoroethyl, methoxyethoxymethyl, trifluoromethoxymethyl, methyl-(O)ₙS, aryl, isoxazolinyl, morpholinyl or methyl-(O)ₙS-amino, where the heterocyclyl groups and aryl groups are substituted by s radicals from the group consisting of methyl, trifluoromethyl and chlorine;
A represents a direct bond or (C₁-C₄)-alkylene;
R⁵ represents (C₁-C₆)-alkyl, (C₃-C₆)-cycloalkyl, (C₃-C₆)-cycloalkyl-(C₁-C₆)-alkyl, (C₁-C₆)-alkoxy-(C₁-C₆)-alkyl;
X¹ represents CR⁶;
X² represents CR⁷;
X³ represents CR⁸;
R⁶ and R⁷ represent hydrogen;
R⁸ represents hydrogen, halogen, (C₁-C₆)-alkyl, halo-(C₁-C₆)-alkyl, (C₂-C₆)-alkenyl, (C₂-C₆)-alkynyl or (C₁-C₆)-alkyl-(O)ₙS;
n represents 0, 1 or 2;
s represents 0, 1, 2 or 3.

4. The 2-(hetero)arylpyridazinone or salt as claimed in claim 1 in which
R¹ represents methyl or vinyl;
R² represents hydrogen;
R³ represents hydrogen;
R⁴ represents methyl, chlorine, trifluoromethyl or methyl-(O)ₙS;
A represents a direct bond, —CH₂— or —CH₂CH₂—;
R⁵ represents methyl, ethyl, cyclopropyl, cyclopropylmethyl, methoxyethyl;
X¹ represents CR⁶;
X² represents CR⁷;
X³ represents CR⁸;
R⁶ and R⁷ represent hydrogen,
R⁸ represents methyl, ethyl, chlorine, trifluoromethyl or methyl-(O)ₙS;
n represents 0, 1 or 2.

5. A herbicidal composition comprising a herbicidally active content of at least one compound of the formula (I) or salt as claimed in claim 1.

6. The herbicidal composition as claimed in claim 5 in a mixture with one or more formulation auxiliaries.

7. The herbicidal composition as claimed in claim 5, comprising at least one further pesticidally active substance from the group consisting of insecticides, acaricides, herbicides, fungicides, safeners, and growth regulators.

8. A method for controlling one or more unwanted plants, comprising applying an effective amount of at least one compound of the formula (I) or salt as claimed in claim 1 or of a herbicidal composition thereof to the plants or to a site of unwanted vegetation.

9. A product comprising a compound of the formula (I) or salt as claimed in claim 1 or herbicidal composition thereof adapted for controlling one or more unwanted plants.

10. The product as claimed in claim 9, wherein the compound of the formula (I) or salt is used for controlling unwanted plants in one or more crops of one or more useful plants.

11. The product as claimed in claim 10, wherein the useful plants are transgenic useful plants.
